(19) United States
(12) Patent Application Publication
(10) Pub. No.: US 2024/0327473 A1
(43) Pub. Date: Oct. 3, 2024

(54) TANDEM REPEAT PROTEIN SEQUENCES IN PROTEIN-LIKE POLYMERS AND USES THEREOF

(71) Applicant: Northwestern University, Evanston, IL (US)

(72) Inventors: Nathan C. GIANNESCHI, Wilmette, IL (US); Or BERGER, Evanston, IL (US); Julia OKTAWIEC, Evanston, IL (US)

(21) Appl. No.: 18/153,872

(22) Filed: Jan. 12, 2023

Related U.S. Application Data
(60) Provisional application No. 63/299,572, filed on Jan. 14, 2022.

(51) Int. Cl. C07K 14/435; A61K 47/58; C08F 289/00

(52) U.S. Cl. CPC C07K 14/43504; A61K 47/58; C08F 289/00

(57) ABSTRACT

Disclosed are protein-like polymers and uses thereof. The protein-like polymers generally comprise a polymer of formula (FX1). The polymer of formula (FX1) in some aspects comprises a peptide having similarity or homology to a tandem repeat peptide sequence found in a natural protein or natural peptide, and/or the polymer of formula (FX1) in some aspects comprises a peptide comprising at least one catechol residue.

### CROSS-REFERENCE TO RELATED APPLICATIONS

This application claims the benefit of priority to U.S. Provisional Patent Application No. 63/299,572, filed Jan. 14, 2022, which is hereby incorporated by reference in its entirety.

### STATEMENT REGARDING FEDERALLY SPONSORED RESEARCH OR DEVELOPMENT

This invention was made with government support under Award Number W911NF-15-1-0568 awarded by the Army Research Office (ARO), and under Award Number FA9550-18-1-0142 awarded by the Air Force Office of Scientific Research (AFOSR). The government has certain rights in the invention.

### REFERENCE TO AN ELECTRONIC SEQUENCE LISTING

The content of the electronic sequence listing (339505_95-21_US_ST1.xml; Size: 15,159 bytes; and Date of Creation: Jan. 11, 2023) is herein incorporated by reference in its entirety.

### BACKGROUND

For over two decades researchers have tried to efficiently reproduce tandem repeat proteins (TRPs) of functional and potential technological utility in a large variety of systems. With vastly diverse biological function, amino acid composition and secondary structure content, TRPs are comprised of multiple repetitions of small conserved motifs (typically less than 40 amino acids) arranged in a linear fashion to produce elongated, simple topological architectures such as α-helices or β-sheets. TRPs display a wide array of highly unique mechanical properties, and are prevalent across all life forms. Fibrous scaffolds such as collagen and keratin, extremely flexible biomaterials like elastin and resilin, and the exceptionally sturdy squid ring teeth proteins are a few notable examples, all of which possess potential if harnessed in engineering applications. The expression and purification of silk proteins for instance, has been attempted in bacterial, yeast, plant, and insect cells. Transgenic whole organisms were investigated for the production of these proteins as well, ranging from small species such as silkworms and mice to mammals as large as goats.

Large scale production of tandem repeat proteins is being hindered by challenging production and purification. Undesirable recombination events involving repetitive DNA sequences, formation of inclusion bodies in the host cell and protein misfolding, all ultimately lead to low yield and poor solubility of the product, see Heidebrecht & Scheibel, Recombinant Production of Spider Silk Proteins, Advances in Applied Microbiology, Academic Press Inc., 2013, Vol. 82, 115-153. The need to use an expression system lacking the right post-translational modifications found in the native system, for instance the conversion of tyrosine to DOPA, can also prevent the protein from being functional. We propose a synthetic approach in which the production and purification of protein-like materials is facile, generic and straightforward.

Our group has previously described a method for formulating peptides into highly dense brushes that provide proteolytic resistance as well as retained bioactivity, see e.g., PCT patent application PCT/US22/23274, which is hereby incorporated by reference. Computational models of these macromolecules showed that they fold similarly to globular proteins, therefore we term them ‘protein-like polymers’ (PLPs). Since the PLPs exhibit a dense array of the functional entity, they can have enhanced properties compared with a linear version in which the majority of the sequence is buried in the hydrophobic core. The synthesis and purification are relatively simple, cost effective and can be automated and scaled up. Moreover, the process can be generalized to any repeating sequence.

In the present disclosure, the tandem repeat unit of the mussel foot protein is used as a model peptide. Mussels produce one of the strongest water insoluble glues. The mussel's adhesive is a bundle of threads, called a byssus, formed by a fibrous collagenous core coated with adhesive proteins. The adhesive byssus provides the mussel with the ability to attach to a variety of solid substrates in the sea. The extraordinary robust adhesion in a turbulent wet and salt-rich environment has attracted much attention and many studies were directed toward mimicking the mussel adhesion. There are over eight different proteins that are secreted from the mussel foot and responsible for coating and adhesion of the mussel byssus. All of which contain tandem repeats of 5-15 amino acids in which one or more residues are 3,4-dihydroxyphenylalanine (L-DOPA), a catecholic amino acid that is formed by post-translational modification of tyrosine, which is believed to play key role in the marine adhesion.

The most studied mussel adhesive protein is foot protein 1 of the genus Mytilus edulis (Mefp-1). It is comprised of over 80 repeats of the decapeptide Ala-Lys-Pro-Ser-Tyr-Hyp-Hyp-Thr-DOPA-Lys (Hyp=hydroxyproline), which encompass most of the protein sequence. Here we report the synthesis of a mussel adhesive-inspired proteomimetic. The underwater glue produced by marine mussels is composed of a highly repetitive short amino acid sequence motif rich in the catecholic amino acid L-DOPA. Polymerization of the repeating unit in the form of a brush polymer, rather than arranged in a linear fashion as in the natural protein, offers high density and surface availability. The synthetic polymer was examined and compared to the native protein in a series of assays including mechanical measurements of adhesion forces at the contact between solid surfaces as well as immobilization of live cells. The results reveal improved adhesion properties over the natural protein which make it attractive for diverse medical and technological applications, such as underwater adhesion, surgical adhesion, dental adhesion, embryo adhesion for in vitro fertilization, co-polymerization with therapeutic peptides for slow release at the site of action, and as an immobilization agent for cells and tissues.

As an additional model peptide, the tandem repeat unit of ankyrin proteins is disclosed herein, specifically incorporating the helical ankyrin repeat. These tandem repeat proteins are known to fold into elongated, non-globular structures that can display high stability. Taking advantage of these properties, synthetic proteins based on ankyrin repeats, called “designed ankyrin repeat proteins,” are under investigation as antibody mimetics and PPI-disrupting drugs for macular degeneration and breast cancer.

The present invention provides an alternative to the labor-intensive needed to express and purify the tandem repeat protein, which has been a significant hurdle to accessing, optimizing and using these proteins. As the system can be generalized to any tandem repeat sequence in principle, a myriad of functional biomaterials can be achieved via this approach. Furthermore, the ease of synthesis also makes this system ideal to readily test and compare changes in the tandem sequence to obtain improved physio-mechanical properties and to inform efforts to develop and optimize engineered protein-based analogues.

### SUMMARY OF THE INVENTION

In an aspect, the invention provides a polymer characterized by a formula (FX1):

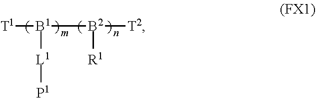

wherein at least one P¹ independently comprises a peptide; at least one P¹ comprises: (i) a sequence having at least 75% or greater sequence identity (e.g., 75% or greater, 80% or greater, 85% or greater, 90% or greater, 95% or greater, 99% or greater, or 100% sequence identity) of a tandem repeat peptide sequence found in a natural protein or natural peptide and/or (ii) at least one catechol residue; T¹ and T² are polymer backbone terminating groups that can be the same or different; B¹ and B² are each independently polymer backbone subunits; each L¹ is independently a linking group; each R¹ is independently a substituent; m is an integer selected from the range of 2 to 1000; n is an integer selected from the range of 0 to 1000; each connecting line in formula (FX1) represents a covalent linkage comprising at least one of a single bond, a double bond, one or more atoms, or any combination thereof, optionally, for example, each connecting line represents a single bond or double bond; each instance of B¹, B², L¹, R¹, and P¹ is the same as or different from any other instance of B¹, B², L¹, R¹, and P¹, respectively; and when n is an integer from 1 to 1000 and/or at least one instance of P¹ is different from another instance of P¹, the polymer is a block copolymer or a statistical copolymer.

In some aspects, at least one P¹ of formula (FX1) comprises (i) a sequence having at least 60% or greater sequence identity (e.g., 60% or greater. 65% or greater, 70% or greater, 75% or greater, 80% or greater, 85% or greater, 90% or greater, 95% or greater, 99% or greater, or 100% sequence identity) of a tandem repeat peptide sequence found in a natural protein or natural peptide, and/or (ii) at least one catechol residue.

In other aspects, the present invention comprises an adhesive composition comprising any one of the polymers described herein.

The present invention further includes a method of adhering a first substrate to a second substrate comprising disposing any one of the polymers described herein between the first substrate and the second substrate.

Further disclosed herein is a method of making any one of the polymers described herein, the method comprising: synthesizing at least one P¹ peptide; capping the at least one P¹ peptide at a terminal end with a polymerizable monomer that, once polymerized, becomes polymer backbone subunit B¹, thereby forming a polymerizable P¹ monomer; and polymerizing the polymerizable P¹ monomer.

The present invention further includes a method of using any one of the polymers described herein as a therapeutic, as an antibody mimetic, as a protein-protein interaction (PPI) disrupting agent, or any combination thereof.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D: The naturally occurring tandem repeats and the synthetic proteomimetic brush polymer. Cartoon representation of the comparison between a linear tandem repeat protein sequence (FIG. 1A) and a PLP (FIG. 1B). The PLP configuration allows for high density display of the functional sequence. FIG. 1C: The full 875-amino acid sequence of Mefp 1, one of the mussel adhesive proteins derived from the mussel's byssus. The decapeptide tandem repeat AKPSYPPTYK is highlighted in darker font for exact repeats and light font for partial or scrambled repeats. Overall the protein contains over 80 repeats of this short sequence. FIG. 1D: Chemical structure of the norbornenyl-Mefp 1 tandem repeat monomer including the post-translational modified amino acids Hyp and DOPA, highlighted by boxes. The norbornene-amino hexanoic acid (NorAha) linker is shown on the leftmost portion of the norbornenyl-Mefp 1 TRP monomer structure.

FIGS. 2A-2F: Synthetic route and characterization of the adhesive polymers. FIG. 2A: ROMP was employed to generate PLPs from norbornenyl-peptide monomers. FIG. 2B: The Mefp 1 tandem repeat peptide monomer and initiator were mixed in different ratios to give three different molecular weights (Small(S), Medium (M) and Large (L)). FIG. 2C: Two control peptide monomers were prepared and polymerized including the Mefp 1 tandem repeat with no post-translational modified amino acids, and (FIG. 2D) a non-functional sequence was utilized of the same length, where only the DOPA residue is similar to the original peptide. FIG. 2E: The different PLPs were characterized by SEC-MALS. FIG. 2F: PLP-S, PLP-M and PLP-L were subjected to SDS-PAGE. Separation according to molecular mass is well correlated with the protein ladder standards.

FIGS. 3A-3C: Adhesion measurements. FIG. 3A: Silicon wafer chips were coated with the different adhesive PLPs or controls and scanned for adhesion force by AFM. Overall 50 different spots were measured for each specimen. The adhesion force distribution for each material is displayed. Our results show no significant difference between the 3 tested degrees of polymerization. The control PLPs show little to no adhesion. FIG. 3B: Schematic illustration of the centrifugal adhesion test. Microparticles added onto adhesive films in a multiwell plate, followed by centrifugation of the plate, causing the particles to detach in correlation to the adhesive properties of the film. FIG. 3C: Comparison of measurements made by the centrifugal adhesion test. The detachment of the microparticles at different centrifugal speeds is represented by a heat map displayed on squares that represent the adhesive coated wells across the multi-well plate. Increasing the centrifugal speed revealed better adhesion at higher degrees of polymerization for the PLPs.

FIGS. 4A-4C: Cell immobilization. FIG. 4A: Three cell lines with different intrinsic adherence strength were cultured in plates coated with either PLP-M or Mefp 1. Following rinsing and staining for live cells, the plates were imaged. Increased fluorescence can be observed in the wells coated with the PLP. The images show the entire well bottoms of a 24-well multiplate. FIG. 4B: Zoomed in images of adhered cells of the three examined lines on PLP-M coating (40× magnification). FIG. 4C: The fluorescence images were analyzed to give the number of cells in each well. The number of cells in untreated wells that were not rinsed is given as an indication of the initial number of cells in each well.

FIG. 5: Measurements made by the centrifugal adhesion test, grouped by material. Each bar represents an average of 6 wells.

FIGS. 6A-6D: Analytical HPLC traces of the peptide and peptide monomers used for polymerization.

FIGS. 7A-7D: ESI-MS spectra of the peptide and peptide monomers used for polymerization.

FIG. 8: Polymerization kinetics for PLP-S/M/L as determined by 1H-NMR in DMF-d7.

FIG. 9: Polymerization kinetics for PLP-Y as determined by 1H-NMR in DMF-d7.

FIG. 10: Polymerization kinetics for PLP-GS as determined by 1H-NMR in DMF-d7.

FIG. 11: SEC-MALS traces (light scattering detector) of Mefp-1 polymers in DMF.

FIG. 12: SEC-MALS traces (light scattering detector) of Mefp-1 and control polymers in DMF.

FIG. 13A: Crystal structure of a single ankyrin repeat from a consensus-derived sequence, showing its helix-loop-helix motif (1NOR). FIG. 13B: Crystal structure of a designed ankyrin repeat protein consisting of seven ankyrin domains (1MJ0). FIG. 13C: Proposed proteomimetic strategy for ankyrin repeat proteins, with peptides displayed on a polymer backbone (based off of 1NOR).

FIG. 14A: Protein-like polymers are flexible brush polymers with tunable globularity and a multivalent display of peptides with proteolytic resistance. They have demonstrated promise for therapeutic applications. FIG. 14B: Scheme showing PLP synthesis by ROMP, as well as the synthesis of block polymers with lengths of different monomers (i.e. other peptides or dyes).

FIG. 15A: Secondary structure of peptides used to construct PLPs of differing lengths (15- to 45-mers), including helical, β-hairpin, and random coil motifs, with corresponding CD data showing similarities between peptides and PLPs. FIG. 15A (left) depicts data for helical PLPs with SEQ ID NO: 11. FIG. 15A (right) depicts data for β-Hairpin PLPs with SEQ ID NO: 12. FIG. 15A (cont.) depicts data for random coil PLPs with SEQ ID NO: 13. FIG. 15B: Normalized Kratky plots of SAXS data showing differences in solvated structure depending on the PLP composition and solvent. FIG. 15B depicts two graphs showing helical PLPs extended in 50% ACN (left) and globular in H₂O (right). FIG. 15B (cont.) depicts two graphs wherein the leftmost graph shows β-Hairpin PLPs as highly globular and the rightmost graph shows floppy PLPs as extended with some globularity.

FIG. 16: 3D models of PLPs to be synthesized by polymerization of consensus-derived ankyrin repeat sequences (FIGS. 16A-16B). Mutations of certain positions, bolded, will be pursued. PLPs will be characterized using CD, SAXS, DSC, and DLS, as well as using cell studies to probe their internalization into cells. VT-CD data shown (FIG. 16C, left) is for helical PLPs of FIG. 16B; example cell images are of PLP uptake (labelled with rhodamine) in neuronal cells (FIG. 16C, right).

FIG. 17: Scheme of Example 5 (i.e., Aim 2) outlining the PPI between p16 and cdk4 being studied and mimicked (1BI7, with cdk6), the proposed peptides that will be studied as monomers and PLPs, the proposed experiments, and goals. Example of PLP is shown (small fragments to the left depict peptide monomer, with larger helices on the right showing 20-amino acid fragment).

FIG. 18: Summary of Example 4 (i.e., Aim 1) (detailed in FIGS. 16A-16B) and Example 5 (i.e., Aim 2) (detailed in FIG. 17) providing an overview of the systematic polymer-based approach to further the generation of the adhesive PLPs disclosed herein.

FIG. 19: MALDI-MS results of H₂N-Ank (FIG. 19A) and NorAha-Ank (FIG. 19B) confirming presence of each peptide in purified product.

FIG. 20: Purification results of the H₂N-Ank (FIG. 20A) and NorAha-Ank (FIG. 20B) showing no appreciable impurities.

FIG. 21: Circular dichroism spectroscopy results showing a helical signal for natural ankyrin peptide monomer.

FIG. 22: Exemplary melting temperature results of 3-repeat and 4-repeat natural ankyrin peptides adapted from a consensus sequence (FIG. 22A) compared to melting temperature results of purified ankyrin peptide monomer (FIG. 22B).

FIG. 23 and FIG. 24: Circular dichroism spectroscopy of the purified ankyrin peptide monomer at elevated and variable temperatures.

FIG. 25: Melting temperature results of purified ankyrin peptide monomer at variable rates of temperature change.

FIG. 26: Polymerization kinetics for PLP-ankyrin as determined by 1H-NMR (FIG. 26A) depicting the percent of ankyrin monomer consumed during polymerization (FIG. 26B).

FIG. 27: ROMP was employed to generate PLPs from norbornenyl-peptide monomers. Resulting PLP-Ank (i.e., NorAnk-no repeat of ankyrin monomer), PLP-Ank₅ (i.e., Ank₅-five repeats of ankyrin monomer), and PLP-Anko (i.e., Ank₁₀-ten repeats of ankyrin monomer) were subjected to SDS-PAGE. FIG. 27 depicts molecular mass separation correlated with the protein ladder standards.

FIG. 28: Circular dichroism spectroscopy results showing signals for PLP-Ank and PLP-Ank₁₀ in two buffers, acetate buffer and TFA (trifluoroacetic acid).

### STATEMENTS REGARDING CHEMICAL COMPOUNDS AND NOMENCLATURE

The following abbreviations are used herein: SPPS refers to solid phase peptide synthesis; ROMP refers to ring-opening metathesis polymerization; RAFT refers to reversible addition fragmentation chain transfer polymerization; DMF refers to dimethylformamide; TFA refers to trifluoroacetic acid; TIPS refers to triisopropyl silane; DTT refers to dithiothreitol; LJ refers to Lennard-Jones; RP-HPLC refers to reverse-phase high performance liquid chromatography; ESI-MS refers to electrospray ionization mass spectrometry; NMR refers to nuclear magnetic resonance spectrometry; MALDI-MS refers to matrix-assisted laser desorption/ionization mass spectrometry; SEC-MALS refers to size-exclusion chromatography coupled with multiangle light scattering; GPC refers to gel permeation chromatography; SDS-PAGE refers to sodium dodecyl sulfate-polyacrylamide gel electrophoresis; CD refers to circular dichroism; SAXS refers to Small-angle X-ray scattering; ARE refers to antioxidant response element; BSA refers to bovine serum albumin; tBHQ refers to tert-Butylhydroquinone; PLP refers to protein-like polymer; NP refers to nanoparticle; PDI refers to polydispersity index; MW refers to molecular weight; and DP refers to degree of polymerization.

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush polymers described herein including the peptide brush and block copolymers and brush and brush block copolymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g., greater than or equal to 1 kDa, in some embodiments greater than or equal to 5 kDa or greater than or equal to 50 kDa).

Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, or 6 or more monomer subunits), and include random, block, brush, brush block, alternating, segmented, grafted, tapered and other architectures. In some embodiments, copolymers of the invention comprise from 2 to 10 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. In embodiments, the invention provides polymers comprising therapeutic agents, such as brush polymers having at least a portion of the repeating units comprising polymer side chains such as peptide side chains.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers, or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

An “oligomer” refers to a molecule composed of repeating structural units connected by covalent chemical bonds often characterized by a number of repeating units less than that of a polymer (e.g., equal to or less than 3 repeating units) and a lower molecular weights (e.g., less than or equal to 1,000 Da) than polymers. Oligomers may be the polymerization product of one or more monomer precursors.

A “peptide” or “oligopeptide” herein refer to a polymer of repeating structural units connected by peptide bonds, including, for example, polypeptides. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein. In some embodiments, a peptide has a chain length of 3 to 150 amino acids, optionally 3 to 100 amino acids, optionally 5 to 50 amino acids, and optionally 5 to 30 amino acids.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A] [B]), or may be provided in a selected sequence ([A] [B] [A] [B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Statistical copolymers,” also generally known in the art as “random copolymers,” are copolymers in which the ordering of backbone groups is dictated by reaction kinetics and comprise spatially randomized units, wherein at least two chemically distinguishable polymerized monomers are randomly distributed throughout the polymer. Statistical copolymers generally are antithetical to block copolymers.

“Polymer backbone group” or “polymer backbone subunit” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R¹⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl. In some embodiments, polymer backbones may terminate in backbone terminating groups including hydrogen, C₁-C₈ alkyl, C₃-C₈ cycloalkyl, C₅-C₈ aryl, C₅-C₈heteroaryl, C₁-C₅ acyl. In some embodiments, polymer backbones may terminate in backbone terminating groups including hydrogen, C₁-C₃ alkyl.

“Polymer side chain group” (also sometimes referred to herein as “substituent,” e.g., with respect to R¹) refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen or C₁-C₅ alkyl.

As used herein, the term “responsive” refers to an agent or a peptide wherein at least a portion of its formulation is capable of interacting with at least a portion of a specific molecule. For example, a responsive peptide may include an amino acid sequence corresponding to a cut-site for a specific enzyme.

As used herein, the term “nonresponsive” refers to an agent or a peptide having a formulation that is not known to interact with a specific molecule.

The term “therapeutic agent” as used herein refers to a class of agents capable of treating or managing a disease, illness, or other condition of a subject. In some embodiments, the therapeutic agent is a pharmaceutical or biological agent or component or fragment thereof. In an embodiment, the therapeutic agent is a therapeutic peptide. In embodiments, the therapeutic agent may be a therapeutic peptide having a chain length of 3 to 150 amino acids, optionally of 3 to 100 amino acids, optionally 5 to 50 amino acids and optionally 5 to 20 amino acids. The therapeutic peptide may be a naturally-occurring peptide, a synthetic peptide, or a purified recombinant peptide. In embodiments, the therapeutic peptide may comprise a TRP sequence found in natural proteins or natural peptides. In other embodiments, the therapeutic agent may be a small molecule therapeutic. In examples, the small molecule therapeutic comprises a low molecular weight organic compound having a size less than or equal to 20 nm, optionally less than or equal to 15 nm, optionally less than or equal to 10 nm. In some embodiments, the therapeutic peptide is characterized by an average molecular weight less than or equal to 40 kDa, optionally less than or equal to 30 kDa, optionally less than or equal to 20 kDa, optionally less than or equal to 10 kDa, and optionally less than or equal to 5 kDa. In some embodiments, the therapeutic peptide is characterized by an average molecular weight of 0.5 kDa to 20 kDa, optionally of 0.5 kDa to 10 kDa and optionally of 1 kDa to 5 kDa.

As used herein, the term “degree of polymerization” refers to the average number of monomer units per polymer chain. For example, for certain polymers described herein, comprising B¹, B², and/or B³ backbone units, the degree of polymerization would be represented by the sum total of B¹, B², and B³ backbone units. Since the degree of polymerization can vary from polymer to polymer, the degree of polymerization is generally represented by an average.

In embodiments, L¹ and L² are linking groups, and optionally a linking group comprising a polymer grafting group. In some embodiments, L¹ and L² independently are optionally functionalized by one or more additional substituents, such as peptide substituents or substituents derived from small molecules. In some embodiments, L¹ and L² independently comprise a linking group selected from the group consisting of a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%. Brush polymers, such as the polymers disclosed herein (e.g., a polymer of formula (FX1)), can be prepared by any suitable methods including, “grafting from” methods, “grafting onto” methods, “grafting through” methods, or any combination thereof. Such suitable methods can include, for example, ring opening metathesis polymerization (ROMP) synthetic pathways and/or non-ROMP synthetic pathways, such as, by way of example, reversible addition fragmentation chain transfer (RAFT) polymerization, stable free radical mediated polymerization and atom transfer radical polymerization (ATRP).

As used herein, the term “peptide density” refers to the percentage of monomer units in the polymer chain which have a peptide covalently linked thereto, and such “peptide density” can be calculated generally for all peptides or for a specific peptide. The percentage is based on the overall sum of monomer units in the polymer chain. For example, for certain polymers described herein, the density of peptide P¹ (or percentage of monomer units comprising peptide P¹) in a polymer having m repeat units of peptide P¹, n repeat units of B²-R¹, and o repeat units of peptide P², is represented by the formula:

$$\frac{m}{m + n + o} \times 100 ,$$

where each variable refers to the number of monomer units of that type in the polymer chain. Polymers of certain aspects are characterized by a peptide density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects are characterized by a peptide density selected from the range 50% to 100%, optionally some embodiments a density selected from the range of 75% to 100%, or optionally for some embodiments a density selected from the range of 90% to 100%. In some embodiments, the brush density is equal to the peptide density.

In an aspect, the polymer side chain groups (e.g., also termed substituents herein) can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6=5 angstroms apart on the polymer backbone. In some embodiments the brush polymer has a high a brush density (e.g., greater than 70%), wherein the polymer side chain groups are spaced 5 to 20 angstroms apart on the polymer backbone.

As used herein, the term “sequence homology” or “sequence identity” means the proportion of amino acid matches between two amino acid sequences of interest in two different peptides considering the ordering of the amino acids. Matches occur when amino acids are in the same order in one peptide compared to the other peptide. When sequence homology is expressed as a percentage, e.g., 50%, the percentage denotes the fraction of matches over the length of sequence that is compared to some other sequence, considering the amino acid order. Gaps (in either of the two sequences) are permitted to maximize matching; for example, wherein gap lengths of 5 amino acids or less, optionally 3 amino acids or less, are usually used. In other words, a sequence having 75% or greater sequence identity to an amino acid sequence with 9 amino acids can indicate that the 9 amino acid sequence can have one or two point mutations (i.e., amino acid change), one or two amino acid deletions, one or two amino acid additions, one point mutation and one amino acid deletion, or one point mutation and one amino acid addition. Even with two such amino acids being different, 7 out of 9 amino acids still match in the correct order, such that there is greater than 75% sequence identity. For clarity, the analysis of whether there sequence homology between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

As used herein, the term “amino acid composition similarity” or “amino acid similarity” means the proportion of amino acid matches between two amino acid sequences of interest in two different peptides regardless of the ordering of the amino acids. Matches occur when amino acids are present in both amino acid sequences regardless of order. When amino acid composition similarity is expressed as a percentage, e.g., 50%, the percentage denotes the fraction of matches over the length of sequence that is compared to some other sequence, regardless of amino acid order. Gaps (in either of the two sequences) are permitted to maximize matching; for example, wherein gap lengths of 5 amino acids or less, optionally 3 amino acids or less, are usually used. By way of example, if two amino acid sequences each containing ten amino acids have three amino acids in common, in any order, then there is 30% amino acid composition similarity between the sequences. For clarity, the analysis of whether there is amino acid composition similarity between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

The term “tandem repeat protein,” also referred to as “TRP,” is known in the art. By way of example, a TRP refers to a protein (e.g., peptide chain) comprising an amino acid sequence (herein termed a “tandem repeat peptide sequence”) that repeats at least 2 times, e.g., at least 3, 4, 5, 6, 7, 8, 9, 10, 11, 12, 13, 14, 15, 16, 17, 18, 19, or 20 times in sequence along the length of the protein. In other words, the tandem repeat peptide sequence is found repeated in series along the protein, in which each tandem repeat peptide sequence optionally is separated by one or more amino acids or other spacers. In some aspects, each tandem repeat peptide sequence in a protein is identical. In other aspects, some tandem repeat peptide sequences in a protein are similar, but not identical, to other tandem repeat peptide sequences in the same protein, and in this case the tandem repeat peptide sequences have “amino acid composition similarity” or “sequence homology” as those terms are defined elsewhere herein.

The terms “natural protein” or “natural peptide” as used herein refer to peptides or proteins that are found in nature. Although such peptides or proteins may be able to be synthesized in a lab setting, natural peptides or proteins were originally discovered in nature, e.g., being produced by natural organisms, such as mussels.

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush copolymers, homopolymers, copolymers, block copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

The term “antibody mimetic” refers to an organic compound with the ability to specifically bind antigens but are not structurally related to antibodies. Typical antibody mimetics are not produced by a subject's immune system and instead are artificially produced. Additionally, antibody mimetics are generally smaller than antibodies and have greater stability. However, it will be understood that antibody mimetics may be synthetically produced to comprise specific properties depending on desired outcome, including variable size, greater stability, greater affinity, protease-resistance and improved solubility. For example, antibody mimetics include peptide aptamers, affitins, avimers, armadillo repeat proteins, designed ankryin repeat proteins (DARPins), and anticalins.

As used herein, the term “compound” can be used to refer to any of the peptides or polymers described herein. Alternatively, or additionally, the term compound can refer to any of the synthetic precursors, reagents, additives, excipients, etc. used in preparation of or formulation with the peptides or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” generally refers to a compound wherein a hydrogen is replaced by another functional group, unless otherwise contradicted by context.

As is customary and well known in the art, hydrogen atoms in formulas (FX1)-(FX2) are not always explicitly shown, for example, hydrogen atoms bonded to the carbon atoms of aromatic, heteroaromatic, and alicyclic rings are not always explicitly shown in formulas ((FX1)-(FX2)). The structures provided herein, for example in the context of the description of formulas (FX1)-(FX2) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 2 to 7 amino acid residues. The 2 to 7 amino acids can be added in a single block containing from 2 to 7 amino acid residues or more than one block containing from 1 to 6 amino acid residues. In preferred embodiments, the charge modulating domain is a cationic residue domain having from 2 to 7 amino acid residues selected from lysine, arginine, histidine, or a combination thereof. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or copolymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or copolymer comprising the peptide is determined by measuring the zeta potential.

Unless otherwise specified, the term “average molecular weight,” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L¹, L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L¹, L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L¹, L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., L¹, L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L¹, L²,).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₈ cycloalkenylene groups, for example, as one or more linking groups (e.g., L¹, L², L³).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L¹, L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy) n-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds. As used herein, “protected amino acids” refer to amino acids in which the amine group and/or the carboxylic acid group are protected by a temporary protecting group. For example, t-butyloxycarbonyl (Boc) and 9-fluorenlmethoxycarbonyl (Fmoc) are temporary protecting groups used in SPPS.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy.

Alkoxy groups include substituted alkoxy groups wherein the alky portion of the groups is substituted as provided herein in connection with the description of alkyl groups. As used herein MeO-refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms.

Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including-CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

Catechol residues, as used herein, are compounds comprising a benzene core having two hydroxyl substituents ortho to each other. Specific catechol derivatives include catechin, piceatannol, catecholamines, dopamine, quercetin, dihydrocaffeic acid, and hydroxyquinol. Formulations of some embodiments of the invention comprise polymer side chains comprising the catecholic amino acid, 3,4-dihydroxy-L-phenylalanine (L-DOPA).

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or(S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both/and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The expression “hydrophilic amino acid residue” refers to an amino acid group, modified amino acid group or substituted amino acid group having at least partial hydrophilic character under at least some conditions, such as in vivo conditions, including arginine, asparagine, aspartate, glutamine, glutamate, or lysine. The expression “neutral amino acid residue”, refers to an amino acid group, modified amino acid group or substituted amino acid group having at least neutral charge character under at least some conditions, such as in vivo conditions, including as histidine, proline, or tyrosine. The polymers of some embodiments comprise peptides having one or more hydrophilic amino acid residues and/or neutral amino acid residues.

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient” “subject” or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., A1-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Nanoparticles (NPs) are a type of nanocarrier (NC) capable of transporting small molecules throughout a subject, providing protection to small molecules from a surrounding environment, protecting the surrounding environment from biological activity of small molecules, and/or targeting delivery of small molecules to a specific site. NPs may be polymeric NPs, which generally have a size between the range of 1 to 1000 nm. NPs are generally categorized as nanospheres or nanocapsules. See Zielińska et al., Molecules, 25:3731 (2020). As used herein, NPs configured to transport therapeutic agents are referred to as “drug-loaded NPs.”

### DETAILED DESCRIPTION

In the following description, numerous specific details of the polymers, polymer components, compositions, and methods of the present invention are set forth in order to provide a thorough explanation of the precise nature of the invention. It will be apparent, however, to those of skill in the art that the invention can be practiced without these specific details.

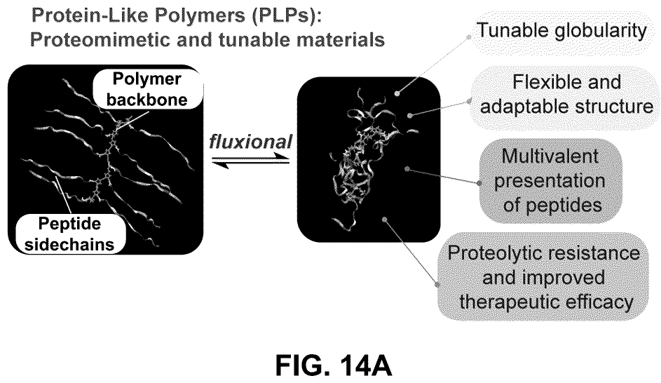

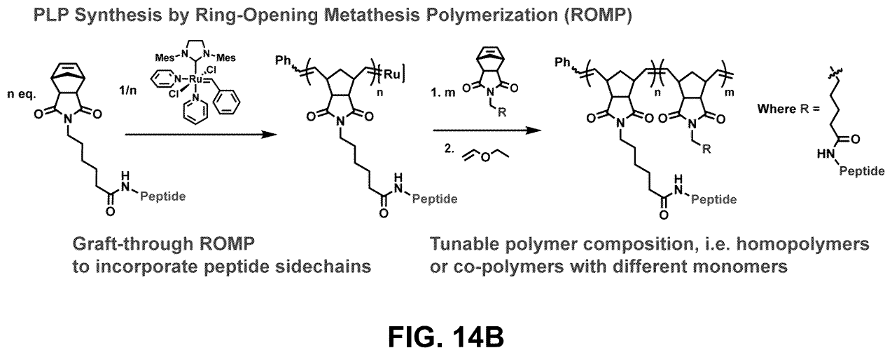

The present invention provides a platform for mimicking tandem repeat proteins (TRPs) through the use of protein-like polymers (PLPs, FIGS. 14A-14B). PLPs are brush polymers synthesized via graft-through polymerization of peptide-based monomers, resulting in peptide side-chains tethered to a hydrocarbon backbone, i.e. poly(norbornene) (FIGS. 14A-14B). By covalently attaching peptides to the polymer, their proteolytic resistance increases relative to the peptide by itself. Additionally, PLPs display multivalency and avidity of binding, due to the presentation of multiple binding sites allowing enhanced interactions. As a result of these promising features, such polymers are under examination for treatments of neurodegenerative diseases, ocular damage, and cancer (see PCT/US20/45729 and Ser. No. 15/502,166). Despite being comprised of natural amino acids, PLPs exhibit high resistance to proteolysis, which is hypothesized to arise from tertiary structure conferred by the artificial polymer backbone.

Herein, brush copolymers incorporating tandem repeats from natural peptides are described. The present invention discloses peptide brush polymers wherein peptides are tethered to a hydrocarbon polymer backbone in a dense display. This results in a topology that resembles repeat proteins, but where the peptides repeats are arranged on a synthetic scaffold. In embodiments, the peptide brush polymers can be made using polymerization to have a targeted number of peptide monomers, i.e., degree of polymerization (DP). It is believed that these proteomimetic platforms can enable conformational mimics of TRPs of natural peptides and can adopt the mechanical and functional properties of the natural peptides.

As a model of a synthetic polymer proteomimetic, a brush polymer is described that reconstitutes the key structural elements and function of mussel adhesive protein. In embodiments, the proteomimetic is prepared via graft through ring opening metathesis polymerization (ROMP) of a norbornenyl-peptide monomer. In embodiments, the peptide is derived from the natural underwater glue produced by marine mussels that is composed of a highly repetitive 10 amino acid tandem repeat sequence. To this end, the arrangement of peptide units was as sidechains on a brush polymer rather than arranged in a linear fashion as in the natural protein. As disclosed herein, mechanical measurements of adhesion forces between solid surfaces revealed improved adhesion properties over the natural protein, making this strategy attractive for diverse applications. In aspects of the present invention, one application is as a surface adhesive for the immobilization of live cells.

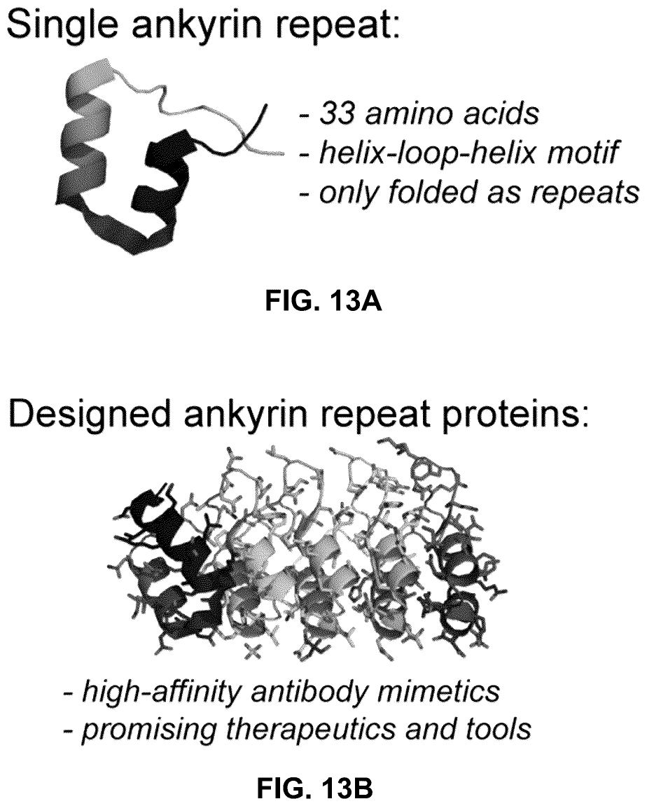

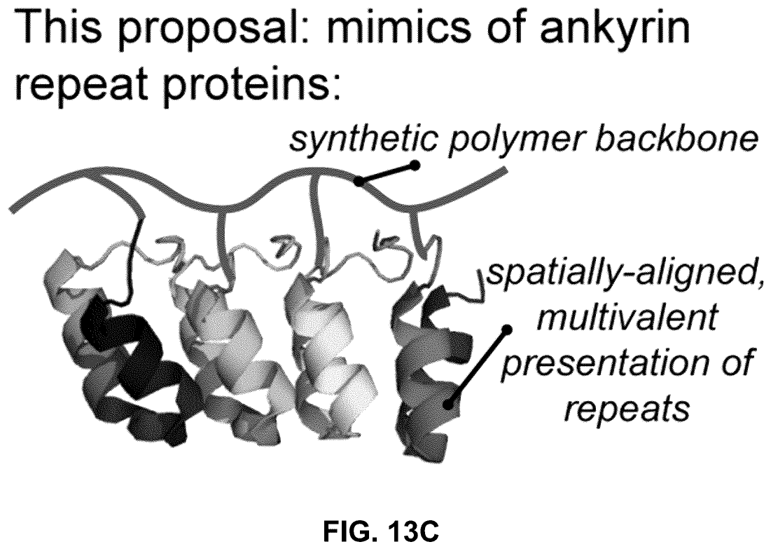

Further, an additional model synthetic polymer proteomimetic is disclosed herein which incorporates tandem repeats from the ankyrin protein family, specifically ankyrin helical repeats. The generic proteomimetic platform disclosed herein enables conformational mimics of ankyrin repeat proteins in order to serve as effective modalities for the disruption of protein-protein interactions (PPIs). For example, the 33-amino acid ankyrin repeat, which consists of a helix-loop-helix motif (FIGS. 13A-13C) acts as a structural scaffold to support a diffuse array of amino acids that can be optimized for particular PPIs within cells and as part of transmembrane receptors. As a result, ankyrin repeat proteins play important roles in many biological processes, ranging from the inflammatory response (IκB-α), to tumor suppression (p16), to the sensation of noxious chemicals (TRPA1). The ability of ankyrin repeat proteins to have specific interactions with a diverse range of proteins is reflected in the development of designed ankyrin repeat proteins (DARPins, FIGS. 13A-13C), which are promising antibody mimetics. Studies of libraries of DARPins have resulted in high-affinity drugs for macular degeneration and breast cancer, which are currently being tested in clinical trials. Studies of engineered ankyrin proteins have also provided insight into native ankyrin repeat proteins, providing information on function challenging to obtain otherwise.

In aspects, a method for mimicking ankyrin repeat proteins via a proteomimetic platform that allows for the facile synthesis of many variations is disclosed. The PLP architecture differs from the linear sequence of amino acids present in proteins. However, ankyrin proteins specifically consist of an elongated and aligned linear series of ankyrin repeats, with the starting position of the repeat adjacent to the starting position of the next repeat (˜8 Å away). As a result, it is hypothesized that the polymerization would result in the positioning of the ankyrin repeats at optimal distances, which is expected to facilitate the folding of the repeat into its helical structure (FIGS. 13A-13C). Therefore, it is anticipated there being an ability to develop accurate structural mimics of ankyrin repeat proteins by efficient polymerization of ankyrin peptides.

Although synthetic polymers cannot be sequence-defined with the same precision as proteins, there is reason to expect that ankyrin-PLPs will display high affinities for particular PPIs due to multivalency. In fact, ankyrins are proposed to have evolved by gene duplication, and thus homo-oligomeric forms of ankyrin repeats should be functional. Modeling an ankyrin repeat protein, p16, as a PLP and studying its interaction with cdk4, cyclin dependent kinase 4, would yield insight for the development of novel cdk4 inhibitors, which are a powerful class of therapeutics used in the treatment of HR-positive breast cancer.

Aspects of the present invention include a polymer platform that might be employed as highly stable and affordable replacements for antibodies, or that could be developed as therapeutics to disrupt disease-relevant PPIs. Additionally, the principles developed for mimicking TRPs could be employed to mimic other repeat classes, including the tetratricopeptide motif, beta-propeller structures, and leucine-rich repeats. More broadly, it would allow for opportunities to use polymer chemistry to accomplish challenging and complex synthetic targets in protein design.

In an aspect, the invention provides a polymer comprising a first polymer segment comprising at least two first repeating units; wherein each of the first repeating units of the first polymer segment comprises a first polymer backbone group directly or indirectly covalently linked to a first polymer side chain group comprising a peptide; wherein the peptide comprises a sequence having 75% or greater (e.g., 80% or greater, 85% or greater, 90% or greater, 95% or greater, or 100%) sequence identity to a TRP sequence found in a natural protein or natural peptide and/or at least one catechol residue. In aspects, the peptide comprises the sequence having at least 75% or greater sequence identity of a TRP sequence found in the natural protein or natural peptide or a modification thereof having at least one catechol residue. The inventive polymer can be any suitable polymer type described herein and can comprise, or be derived from, any suitable number of monomers. For example, in some embodiments, the polymer is a homopolymer (i.e., derived from one type of monomer). Alternatively, in some embodiments, the polymer can be a copolymer comprising (e.g., derived from) more than one type of monomer (e.g., from 2 to 10 types of monomers).

The inventive polymer further includes two polymer backbone terminating groups.

These two polymer backbone terminating groups may be the same or different. Acceptable backbone terminating groups include hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl. acceptable backbone terminating groups include hydrogen, C₁-C₅ alkyl, C₃-C₅ cycloalkyl, C₅-C₈ aryl, C₅-C₈ heteroaryl, C₁-C₅ acyl. In some embodiments, acceptable backbone terminating groups include hydrogen, C₁-C₃ alkyl.

In aspects, the invention provides a polymer comprising a second polymer segment comprising at least one second repeating unit; wherein each of the second repeating units of the second polymer segment comprises a second polymer backbone group covalently linked to a second polymer side chain group comprising a substituent. Each instance of the substituent can be the same or different. Acceptable substituents linked to a second polymer side chain include hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl.

In aspects of the invention, the polymer comprises a third polymer segment comprising at least one third repeating unit; wherein each of the third repeating units of the third polymer segment comprises a third polymer backbone group directly or indirectly covalently linked to a third polymer side chain group comprising a peptide. In these aspects, each instance of a peptide on a first polymer side chain group is the same peptide. Further, each instance of a peptide on a third polymer side chain group is different from the peptides on the first polymer side chains. Additionally, where the polymer comprises more than one third repeating unit, each instance of a peptide on a third polymer side chain group may be the same or different from other instances of peptides on third polymer side chain groups. In some aspects, the peptide on the third polymer side chain group comprises a sequence having 75% or greater (e.g., 80% or greater, 85% or greater, 90% or greater, 95% or greater, or 100%) sequence identity to a TRP sequence found in a natural protein or natural peptide and/or at least one catechol residue. In aspects, the peptide comprises the sequence having at least 75% or greater sequence identity of a TRP sequence found in the natural protein or natural peptide or a modification thereof having at least one catechol residue.

Thus, at least one polymer side chain (e.g., a polymer side chain of the first polymer segment and optionally the third polymer segment) comprises a peptide having 75% or greater (e.g., 80% or greater, 85% or greater, 90% or greater, 95% or greater, or 100%) sequence identity to a TRP sequence found in a natural protein or natural peptide and/or at least one catechol residue. In aspects, the peptide comprises the sequence having at least 75% or greater sequence identity of a TRP sequence found in the natural protein or natural peptide or a modification thereof having at least one catechol residue. The peptide comprises any suitable number of amino acid units so long as the peptide comprises a sequence having 75% or greater (e.g., 80% or greater, 85% or greater, 90% or greater, 95% or greater, or 100%) sequence identity to a TRP sequence found in a natural protein or natural peptide and/or at least one catechol residue. In some aspects, the peptide comprises at least 5 amino acid residues. For example, the peptide comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less amino acid units. Thus, the peptide can comprise a number of amino acid units bounded by any two of the aforementioned endpoints. For example, the peptide can comprise 5 to 100 amino acid units, for example, 5 to 100, 5 to 90, 5 to 80, 5 to 70, 5 to 60, 5 to 50, 5 to 40, 5 to 30, 5 to 20, 5 to 16, 5 to 15, 5 to 14, 6 to 100, 6 to 90, 6 to 80, 6 to 70, 6 to 60, 6 to 50, 6 to 40, 6 to 30, 6 to 20, 6 to 16, 6 to 15, 6 to 14, 7 to 100, 7 to 90, 7 to 80, 7 to 70, 7 to 60, 7 to 50, 7 to 40, 7 to 30, 7 to 20, 7 to 16, 7 to 15, 7 to 14, 8 to 100, 8 to 90, 8 to 80, 8 to 70, 8 to 60, 8 to 50, 8 to 40, 8 to 30, 8 to 20, 8 to 16, 8 to 15, 8 to 14, 9 to 100, 9 to 90, 9 to 80, 9 to 70, 9 to 60, 9 to 50, 9 to 40, 9 to 30, 9 to 20, 9 to 16, 9 to 15, 9 to 14, 10 to 100, 10 to 90, 10 to 80, 10 to 70, 10 to 60, 10 to 50, 10 to 40, 10 to 30, 10 to 20, 10 to 16, 10 to 15, 10 to 14, 11 to 16, 11 to 15, 11 to 14, 12 to 20, 12 to 16, 12 to 15, or 12 to 14 amino acid units. In some embodiments, the peptide comprises 10 to 33 amino acids. In certain embodiments, the peptide comprises 10 to 15 amino acids or 12 to 14 amino acids.

As disclosed herein, the inventive polymer comprises a brush polymer. In aspects, the repetition of peptide motifs found in natural proteins or peptides is accomplished through polymerization of a polymer segment having a polymer side chain group comprising a TRP sequence (e.g., the first polymer segment comprising at least two first repeating units, wherein each of the first repeating units of comprises a first polymer backbone group linked to a first polymer side chain group comprising a peptide; wherein the peptide comprises a sequence having 75% or greater (e.g., 80% or greater, 85% or greater, 90% or greater, 95% or greater, or 100%) sequence identity to a TRP sequence found in a natural protein or natural peptide and/or at least one catechol residue). A targeted number of repeats (i.e., degrees of polymerization) of a polymer segment comprising a sequence having 75% or greater identity to a TRP sequence peptide is believed to contribute to a platform suited for mimicking natural peptides having TRP sequences. The inventive polymer disclosed herein may comprise 1 to 1000 repeats (i.e., degrees of polymerization) of a polymer segment comprising a sequence having 75% or greater (e.g., 80% or greater, 85% or greater, 90% or greater, 95% or greater, or 100%) sequence identity of a TRP sequence found in a natural protein or natural peptide. For example, the polymer may comprise at least 2 repeats, at least 3 repeats, at least 4 repeats, at least 5 repeats, at least 6 repeats, at least 10 repeats, at least 11 repeats, at least 15 repeats, at least 19 repeats, at least 20 repeats, at least 30 repeats, at least 45 repeats, at least 60 repeats, at least 70 repeats, at least 80 repeats, at least 90 repeats, at least 100 repeats, at least 200 repeats, at least 500 repeats, or at least 900 repeats of a polymer segment.

Additionally, in aspects the polymer comprises a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects have a brush density selected from the range 50% to 100%, optionally some embodiments a density selected from the range of 75% to 100%, or optionally for some embodiments a density selected from the range of 90% to 100%. Brush polymers of certain aspects have a “high brush density” selected from the range 90% to 100%, optionally some embodiments a density selected from the range of 95% to 100%, or optionally for some embodiments a density selected from the range of 99% to 100%. For example, in aspects of the invention, the polymer may be characterized by a formulation wherein 90% of its polymer segments comprise a polymer backbone group covalently linked to a polymer side chain group, wherein each polymer segment may comprise the same or different polymer side chain group. In aspects, said polymer side chain group may comprise a sequence having 75% or greater sequence identity of a TRP sequence found in a natural protein or natural peptide and/or at least one catechol residue. In some embodiments, the brush density of the polymer is equal to the peptide density of a particular peptide (e.g., all polymer segments of the polymer comprising polymer backbones covalently linked to a polymer side chain comprising P¹). In other aspects, the brush density of the polymer is different from the peptide density of a particular peptide (e.g., at least one polymer segment comprises a polymer backbone covalently linked to a polymer side chain comprising P¹ and at least one other polymer segment comprises a polymer backbone covalently linked to a polymer side chain comprising P² wherein P¹ and P² are characterized by different sequences).

In aspects, the polymer is characterized by a P¹ peptide density selected from the range 50% to 100%, optionally some embodiments a density selected from the range of 75% to 100%, or optionally for some embodiments a density selected from the range of 90% to 100%. In aspects, the polymer is characterized by a P² peptide density selected from the range 50% to 100%, optionally some embodiments a density selected from the range of 75% to 100%, or optionally for some embodiments a density selected from the range of 90% to 100%.

The peptide can have any suitable structure (e.g., primary, secondary, tertiary, or quaternary structure) described herein. The peptide can be a branched peptide, a linear peptide, cyclic peptide, or a cross-linked peptide. In some embodiments, the polymer is characterized by a structure wherein at least a portion of the peptide is linked to the polymer backbone group via a linker. In other embodiments, the polymer is characterized by a structure wherein the peptide is directly linked to the polymer backbone group.

In some aspects, the peptide comprises a sequence having hydrophobic regions, such as leucine-rich regions. In aspects, the hydrophobic regions may be modified to substitute hydrophilic amino acid residues or non-hydrophobic amino acid residues. It is believed that such substitutions may facilitate improved solubility of the polymer if necessary for certain applications. In aspects where superior stability is desired, the polymer may also be modified. Acceptable polymer modifications include asparagine β-hydroxylation, higher degrees of polymerization (e.g., greater than 10 DP, greater than 15 DP, greater than 30 DP, greater than 45 DP, or greater than 60 DP), single point mutations, and other suitable modifications.

Additionally, the peptide may comprise one or more gaps in its sequence. For example, the at least one peptide may comprise 5 consecutive amino acid residues which do not impact, or do not contribute to, the properties of the at least one peptide's sequence. In aspects, the one or more gaps is a spacer molecule, 5 or less amino acid residues, 3 or less amino acid residues, or a combination thereof.

In some aspects, the peptide (e.g., at least one of P¹ and/or at least one of P² of the polymers characterized in (FX1) and (FX2)) comprises a tandem repeat peptide sequence having at least one lysine residue. In aspects, the TRP sequence consists of any combination of alanine, lysine, proline or 4-hydroxyproline, serine, threonine, or 3,4-dihydroxyphenylalanine or tyrosine, not necessarily in this order. In keeping with this aspect, the TRP sequence consists of the following ten amino acids in any order: alanine, lysine, proline or 4-hydroxyproline, serine, tyrosine or 3,4-dihydroxyphenylalanine, 4-hydroxyproline or proline, 4-hydroxyproline or proline, threonine, 3,4-dihydroxyphenylalanine or tyrosine, and lysine.

In some aspects, the peptide (e.g., at least one of P¹ and/or at least one of P² of the polymers characterized in (FX1) and (FX2)) comprises a sequence having 60% or greater (e.g., 65% or greater, 70% or greater, 75% or greater, 80% or greater, 85% or greater, 90% or greater, 95% or greater, or 100%) sequence identity of a TRP sequence found in a natural protein or natural peptide. In embodiments, the peptide (e.g., at least one of P¹ and/or P² of the polymers characterized in (FX1) and (FX2)) comprises a sequence having 80% or greater (e.g., 85% or greater, 90% or greater, 95% or greater, 99% or greater, or 100%) sequence identity of a TRP sequence found in a natural protein or natural peptide.

In certain aspects of the inventive polymer wherein the polymer is expected to exhibit adhesive properties, the peptide (e.g., at least one of P¹ and/or at least one of P² of the polymers characterized in (FX1) and (FX2)) comprises a sequence having 75% or greater (e.g., 85% or greater, 90% or greater, 95% or greater, 99% or greater, or 100%) sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine. In aspects, the sequence has 75% or greater (e.g., 80% or greater, 85% or greater, 90% or greater, 95% or greater, or 100%) sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine. In keeping with this aspect, the peptide may comprise a sequence having 75% or greater (e.g., 80% or greater, 85% or greater, 90% or greater, 95% or greater, 99% or greater, or 100%) sequence identity of SEQ ID NO: 16 (AKPSYPPTYK).

In aspects wherein the polymer is expected to exhibit adhesive properties, the tandem repeat peptide sequence comprises at least one catechol residue. In certain embodiments, the at least one catechol residue is a 3,4-dihydroxyphenylalanine (L-DOPA), a catecholic amino acid that is formed by post-translational modification of tyrosine. Without binding to a particular theory, it is believed that DOPA-rich content facilitates adhesive properties of TRPs.

In certain aspects of the inventive polymer wherein the polymer supports incorporation of ankyrin repeat sequences, the peptide (e.g., at least one of P¹ and/or at least one of P² of the polymers characterized in (FX1) and (FX2)) comprises a sequence having a basic helix-loop-helix motif (bHLH). In these aspects, the bHLH comprises two alpha-helices connected by a loop. Without subscribing to any particular theory, it is believed that this structural motif facilitates high binding specificity for PPIs, which may be optimized for particular PPIs within cells and as part of transmembrane receptors. In aspects, the direct or indirect linkage between the peptide and the polymer backbone is positioned near one of the alpha-helices of the peptide. In other aspects, the linkage is closer to the loop extension of the peptide.

In keeping with this aspect, the peptide may comprise a sequence having 75% or greater (e.g., 80% or greater, 85% or greater, 90% or greater, 95% or greater, 99% or greater, or 100%) sequence identity of SEQ ID NO: 2 (DKXGRTPLHLAARXGHLEVVKLLLXXGADVNAK), wherein each X independently is a hydrophilic amino acid residue, such as arginine, asparagine, aspartate, glutamine, glutamate, or lysine. In aspects, each X independently is a hydrophilic amino acid residue or a neutral amino acid residue, such as histidine, proline, or tyrosine.

In aspects of the inventive polymer wherein the polymer supports incorporation of ankyrin repeat sequences, the peptide (e.g., at least one of P¹ and/or at least one of P² of the polymers characterized in (FX1) and (FX2)) comprises a sequence having 75% or greater (e.g., 80% or greater, 85% or greater, 90% or greater, 95% or greater, 99% or greater, or 100%) sequence identity of SEQ ID NO: 3 (DKNGRTPLHLAARNGHLEVVKLLLEAGADVNAK).

In other aspects of the inventive polymer wherein the polymer supports incorporation of ankyrin repeat sequences, the peptide (e.g., at least one of P¹ and/or at least one of P² of the polymers characterized in (FX1) and (FX2)) comprises a sequence having 75% or greater (e.g., 80% or greater, 85% or greater, 90% or greater, 95% or greater, 99% or greater, or 100%) sequence identity of SEQ ID NO: 4 (DXXGXTPLHLAAXXGHLEIVEVLLXAGADVNAX), wherein each X independently is a hydrophilic amino acid residue, such as arginine, asparagine, aspartate, glutamine, glutamate, or lysine. In aspects, each X independently is a hydrophilic amino acid residue or a neutral amino acid residue, such as histidine, proline, or tyrosine.

In keeping with this aspect of the inventive polymer, the peptide (e.g., at least one of P¹ and/or at least one of P² of the polymers characterized in (FX1) and (FX2)) may comprise a sequence having 75% or greater (e.g., 80% or greater, 85% or greater, 90% or greater, 95% or greater, 99% or greater, or 100%) sequence identity of SEQ ID NO: 5 (DKNGRTPLHLAARNGHLEIVEVLLEAGADVNAK).

In other aspects of the inventive polymer wherein the polymer supports incorporation of ankyrin repeat sequences, the peptide (e.g., at least one of P¹ and/or at least one of P² of the polymers characterized in (FX1) and (FX2)) comprises a sequence having 75% or greater (e.g., 80% or greater, 85% or greater, 90% or greater, 95% or greater, 99% or greater, or 100%) sequence identity of SEQ ID NO: 6 (DAAREGFLDTLVVLHRAGAR), SEQ ID NO: 7 (FLDTLVVLHR), SEQ ID NO: 8 (ALPNAPNSYGRRPIQVMMMGSARVAELLLLHGAE), SEQ ID NO: 9 (PNCADPATLTRPVHDAAREGFLDTLVVLHRA), SEQ ID NO: 10 (GSGSGS), SEQ ID NO: 11 (KIABAVBLBAEE), SEQ ID NO: 12 (SWTWENGKWTWK), SEQ ID NO: 13 (GSGSGRGSGSGE), SEQ ID NO: 14 (NGRTPLHLAARNGHLEVVKLLLEAGADVNAKDK) or SEQ ID NO: 15 (PNMADPATLTRPVHDAAREGFLDTLVVLHRA).

In aspects, the tandem repeat peptide sequence is found in a mussel or a secretion thereof, a spider silk, a silk protein, an ankyrin, a collagen, a keratin, an elastin, a resilin, a squid ring teeth protein, a fibroin, or an abduction. In aspects, the tandem repeat peptide sequence is found in a mussel foot protein. In other aspects, the tandem repeat peptide sequence is found in an ankyrin.

It will be understood that the inventive polymer, along with the linked polymer side chains, can have any suitable configuration. In aspects, the inventive polymer is arranged in a non-linear fashion. In aspects of the present invention, the polymer supports the structure of a TRP observed in a natural peptide. In keeping with this aspect, the support may be derived from the positioning of the peptide along the polymer backbone, such as positioning the peptides at optimal distances from each other. For example, adjacent ankyrin repeats are believed to fold into their natural helical structure when positioned approximately 8 Å away from each other. In aspects of the invention, where the polymer comprises at least two repeats of the peptide, the distance between the peptides is between the range of 3 to 30 Å. In some aspects, the distance between the peptides is between the range of 5 and 10 Å. In some aspects of the invention, the distance between the peptides is about 8 Å.

In aspects of the inventive polymer, each instance of the first polymer segment, the second polymer segment, and/or the third polymer segment independently comprise one or more polymer backbone units comprising substituted or unsubstituted norbornene, oxanorbornene, olefin, cyclic olefin, norbornene anhydride, cyclooctene, cyclopentadiene, styrene, acrylamide, and acrylate. In aspects, each instance of the first polymer segment, the second polymer segment, 5 and/or the third polymer segment independently comprises one of the following substructures:

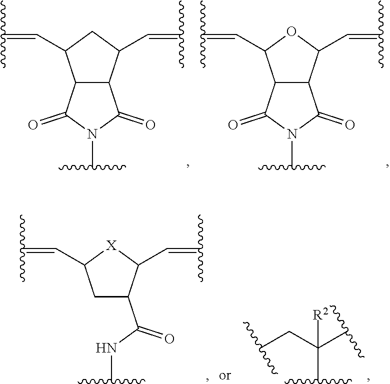

wherein X is CH₂ or O. In further aspects, each instance of the first polymer segment or the third polymer segment independently comprises one of the following substructures:

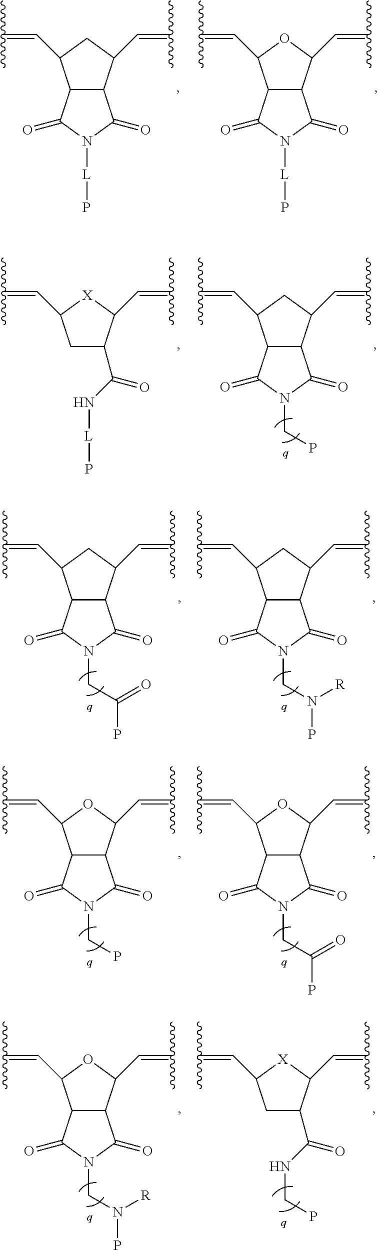

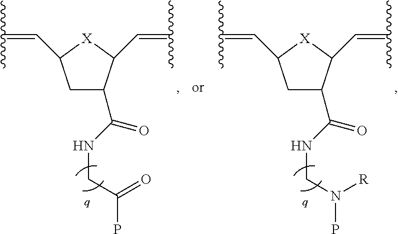

wherein L is a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20; P is optionally present and comprises a peptide, optionally a peptide having 60% or greater (e.g., 65% or greater, 70% or greater, 75% or greater, 80% or greater, 85% or greater, 90% or greater, 95% or greater, 99% or greater, or 100%) sequence identity to a TRP sequence found in a natural protein or natural peptide and/or at least one catechol residue; X is CH₂ or O; q is an integer selected from 1 to 20 (e.g., 1, 2, 3, 4, 5, 6, 7, 8, 9, 10, 11, 12, 13, 14, 15, 16, 17, 18, 19, or 20); and R is hydrogen or a C₁-C₅ alkyl.

In some embodiments, the peptide further comprises a charge modulating domain. The charge modulating domain can be any suitable amino acid domain, which increases the positive charge of the peptide. In some embodiments, the charge modulating domain is a glycine-serine domain, a cationic residue domain, or a combination thereof. In certain embodiments, the charge modulating domain is a cationic residue domain having from 2 to 7 amino acid residues selected from lysine, arginine, histidine, and a combination thereof. In aspects, the peptide sequence comprises arginine residues. In preferred embodiments, the charge modulating domain modulates the peptide to have a net positive charge. In aspects of the present invention, one or more of the polymer side chain groups comprises oligo (ethylene glycol) (OEG) chains, or any other suitable hydrophilic side chain. In some aspects, the peptide comprises cell-penetrating peptide monomers, such as TAT. Without wishing to be bound by any particular theory, it is believed that the net positive charge increases the cellular uptake of the polymer comprising the peptide.

Additionally, for each of the polymers characterized by the formula (FX1) or (FX2) described herein, it will be understood that the first polymer backbone group units, the second polymer backbone group units, and the third polymer backbone group units can be arranged in any suitable order. For example, the first polymer backbone group units, the second polymer backbone group units, and the third polymer backbone group units can be arranged as a random polymer, block polymer, brush, brush block, alternating, segmented, grafted, tapered and other architectures. In other words, variables “m”, “n”, and “o” merely define the total number of that particular monomer in the polymer and do not imply any particular order. For each of the polymers characterized by the formula (FX1) and/or (FX2), each of m, n, and o can be independently selected from any suitable integer. Suitability of the integer is based on the desired DP for each polymer backbone segment as discussed herein.

The polymer backbone groups units (e.g., each of B¹, B², and B³ of the polymers characterized by the formula (FX1), (FX2), and/or the substructures (S1a)-(S2e)), can be independently selected from any suitable polymer backbone subunit. In aspects, each of the first polymer backbone subunit, the second polymer backbone subunit, and the third polymer backbone subunit can be a monomer capable of undergoing ring opening metathesis. For example, each of B¹, B², and B³ can independently be a substituted or unsubstituted norbornene, oxanorbornene, olefin, cyclic olefin, cyclooctene, or cyclopentadiene. In some aspects, each of the first polymer backbone group subunit, the second polymer backbone group subunit, and/or the third polymer backbone group subunit is a polymerized norbornene dicarboxyimide monomer. In some embodiments, each polymer backbone subunit of the polymer is a polymerized norbornene dicarboxyimide monomer. In aspects where the polymer has poor solubility, one or more of the polymer backbone subunits may be substituted with an oxanorbornene-based subunit (if not already in use) or other suitable hydrophilic backbone subunit.

For each of the polymers characterized by the formula (FX1) and (FX2), each of T¹ and T² can be independently selected from any suitable polymer backbone terminating group. For example, hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl.

For each of the polymers characterized by the formulas (FX1) and (FX2) and the substructures (S1a)-(S2e), each of L¹ and L² can be independently selected from any suitable linking group. For example, suitable linking groups include a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, and wherein each instance of L¹ and L² is configured with one or more suitable functional groups to covalently attach a polymer backbone subunit with a peptide, such as an ester or an ether. Each instance of L¹ and L² can be the same or different linking group.

For each of the polymers characterized by the formula (FX1) and (FX2), each R¹ can be independently selected from any suitable substituent. For example, hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR 12R 13, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl. For each of the substructures characterized by (S1c), each R² can be independently selected from hydrogen or C₁-C₃ alkyl.

In some specific embodiments, the polymer comprises one or more peptides and/or proteins other than the peptides having a TRP sequence described herein (i.e., one or more additional peptides and/or proteins). For example, each P¹ and P² polymer segment of formulas (FX1) or (FX2) can independently comprise a peptide or protein other than the TRP peptides described herein.

The one or more additional peptides and/or proteins can be any suitable peptide or protein, having any suitable function. For example, the one or more additional peptides and/or proteins can be an additional peptide having a composition similarity to a TRP sequence found in a natural protein or natural peptide (e.g., an additional TRP peptide described herein), a therapeutic peptide, a cell-penetrating agent (e.g., a cell-penetrating peptide), a targeting agent (e.g., a target-specific peptide to a tissue or cell type), a therapeutically synergistic disease-specific peptide (e.g. a peptide known or thought to be therapeutic for a disease state, such as but not limited to, neurodegenerative disease), an antibody, an antibody mimetic, a protein-protein interaction (PPI) disrupting peptide, or a combination thereof. The additional peptides and/or proteins can be linked to the polymer backbone by any suitable means. In some embodiments, the additional peptides and/or proteins are linked to the polymer backbone via an enzymatically degradable linker (i.e., linking group or linking moiety). Examples of suitable cleavable, degradable or triggerable linkers include enzyme cleavable sequences such as one or more ester bond, reductive sensitive bond-disulfide bond, pH sensitive bond-imine bond, among others.

The one or more additional peptides and/or proteins can have any suitable number of amino acid units. For example, the one or more additional peptides and/or proteins can comprise 2 or more amino acid units, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, or 30 or more amino acid units. Alternatively, or in addition, the one or more additional peptides and/or proteins can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less 33 or less, 32 or less, or 31 or less amino acid units. Thus, the one or more additional peptides and/or proteins can comprise a number of amino acid units bounded by any two of the aforementioned endpoints. For example, the one or more additional peptides and/or proteins can comprise 2 to 100 amino acid units, for example, 2 to 90, 2 to 80, 2 to 70, 2 to 60, 5 to 100, 5 to 90, 5 to 80, 5 to 70, 5 to 60, 3 to 60, 3 to 59, 4 to 58, 5 to 57, 6 to 56, 7 to 55, 8 to 54, 9 to 53, 10 to 52, 11 to 51, 12 to 50, 13 to 49, 14 to 48, 15 to 47, 16 to 46, 17 to 45, 18 to 44, 19 to 43, 20 to 42, 21 to 41, 22 to 42, 23 to 41, 24 to 40, 25 to 39, 26 to 38, 27 to 37, 28 to 36, 29 to 35, 30 to 34, or 31 to 33 amino acid units. In certain embodiments, the one or more additional peptides and/or proteins comprises 5 to 100 amino acids. In preferred embodiments, the one or more additional peptides and/or proteins comprises 8 to 60 amino acid units.

The one or more additional peptides and/or proteins can have any suitable structure (e.g., primary, secondary, tertiary, or quaternary structure). Additionally, the one or more additional peptides and/or proteins can be branched, linear, cyclic, or cross-linked. In some embodiments, the one or more additional peptides and/or proteins is a charge modulating domain. For example, the one or more additional peptides and/or proteins can be or can comprise a TAT sequence, a glycine-serine domain, a cationic residue domain, or a combination thereof, or optionally a glycine-serine domain, a cationic residue domain, or a combination thereof. In certain embodiments, the one or more additional peptides and/or proteins modulates the charge of the polymer to have a net positive charge. Without wishing to be bound by any particular theory, it is believed that the net positive charge increases the cellular uptake of the polymer comprising the peptide.

In some specific embodiments, the polymer comprises a tag for imaging and/or analysis. In aspects, the polymer comprises a fluorescein-, biotin-, or rhodamine-based tag resulting in a fluorescently labelled PLP. For example, each polymer segment B¹, B², or B³ of formula (FX1) or (FX2) can independently comprise a tag for imaging and/or analysis. Similarly, each P¹ or P² of formula (FX1) or (FX2) can independently comprise a tag for imaging and/or analysis. Additionally, each T¹ or T² of any of the formulas described herein can independently comprise a tag for imaging and/or analysis. For example, the polymer can comprise one or more of a dye, a radiolabeling agent, an imaging agent, titration agent, and the like.

The inventive polymers can have any suitable degrees of polymerization. If the degree of polymerization is too low, the polymer may not be resistant to adhesive force or may not be resistant to enzymatic cleavage by proteases or may be cleared too rapidly from the body since the polymer's molecular weight would be lower than the clearance threshold through the kidney. Additionally, if too low, the polymer may exhibit poor solubility and structural instability. Alternatively, if the degree of polymerization is too high, the peptide side chain groups displayed on the polymer may be too dense to engage their biological targets such as cell receptors, enzymes, PPIs etc. Additionally, the high degree of polymerization may result in a polymer that is too large to penetrate cells. Typically, the polymer has a degree of polymerization of 2 to 1000 (e.g., 2 to 500, 2 to 250, 2 to 100, 2 to 60, 2 to 50, 2 to 30, 5 to 1000, 5 to 500, 5 to 250, 5 to 100, 5 to 60, 5 to 50, 5 to 45, 5 to 30, 15 to 45, 20 to 500, 20 to 250, 20 to 100, 20 to 50, or 20 to 30). In certain embodiments, the polymer has a degree of polymerization of 5 to 100. In preferred embodiments, the polymer has a degree of polymerization of 2 to 200. For example, the polymer can have a degree of polymerization of 2 or about 2, 5 or about 5, a degree of polymerization of 10 or about 10 (e.g., 11), a degree of polymerization of 15 or about 15 (e.g., 17), a degree of polymerization of 20 or about 20, a degree of polymerization of 30 or about 30, a degree of polymerization of 50 or about 50, a degree of polymerization of 60 or about 60, a degree of polymerization of 100 or about 100, a degree of polymerization of 150 or about 150, or a degree of polymerization of 200 or about 200. In some embodiments, the polymer has a degree of polymerization of 2 to 50. In certain embodiments, the polymer has a degree of polymerization of at least 5. In other certain embodiments, the polymer has a degree of polymerization of at least 20.

The inventive polymer can have any suitable weight average molecular weight. The polymers can have a weight average molecular weight of 2,000 kDa or less, for example, 1,800 kDa or less, 1,600 kDa or less, 1,400 kDa or less, 1,200 kDa or less, 1,000 kDa or less, 900 kDa, or less, 800 kDa, or less, 700 kDa or less, 600 kDa or less, 500 kDa or less, 250 kDa or less, 100 kDa or less, or 50 kDa or less. Alternatively, or in addition, the polymers can have a weight average molecular weight of 500 Da or more, for example, 1 kDa or more, 5 kDa or more, 10 kDa or more, 20 kDa or more, 30 kDa or more, 50 kDa or more, 55 kDa or more, or 70 kDa or more. Thus, the polymers can have a weight average molecular weight bounded by any two of the aforementioned endpoints. For example, the polymers can have a weight average molecular weight of from 500 Da to 2,000 kDa, from 500 Da to 1,000 kDa, from 500 Da to 500 kDa, from 500 Da to 100 kDa, from 500 Da to 70 kDa, from 500 Da to 50 kDa, 1 kDa to 2,000 kDa, from 1 kDa to 1,000 kDa, from 1 kDa to 500 kDa, from 1 kDa to 100 kDa, from 1 kDa to 50 kDa, from 1 kDa to 70 kDa, 5 kDa to 2,000 kDa, from 5 kDa to 1,000 kDa, from 5 kDa to 500 kDa, from 5 kDa to 100 kDa, from 5 kDa to 50 kDa, from 5 kDa to 70 kDa, 10 kDa to 2,000 kDa, from 10 kDa to 1,000 kDa, from 10 kDa to 500 kDa, from 10 kDa to 100 kDa, from 10 kDa to 50 kDa, or from 10 kDa to 70 kDa.

In another aspect, the invention provides a pharmaceutical composition comprising one or more peptides and/or one or more polymers described herein. In some embodiments, the composition comprises one or more pharmaceutically acceptable excipients. For example, the peptides and/or polymers of the invention can be formulated for parenteral administration, such as intravenous (IV) administration or administration into a body cavity or lumen of an organ. Alternatively, the peptides and/or polymers can be injected intra-tumorally. Formulations for injection will commonly comprise a solution of the peptide and/or polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the peptide and/or polymer in these formulations can vary widely, and will be selected primarily based on fluid volumes, viscosities, body weight, and the like, in accordance with the particular mode of administration selected and the patient's needs. In certain embodiments, the concentration of a peptide and/or polymer in a solution formulation for injection will range from 0.1% (w/w) to 10% (w/w) or about 0.1% (w/w) to about 10% (w/w).

In another aspect, the invention provides an adhesive composition comprising one or more peptides and/or one or more polymers described herein. Formulations of the adhesive composition will commonly comprise a solution of the peptide and/or polymer dissolved in an acceptable carrier. Formulations of the adhesive composition may comprise a liquid, a hydrogel, a film, a lyophilized powder, a lotion, a cream, a paste, a foam, a spray (aerosol), or a patch. Acceptable vehicles and solvents that can be employed may comprise acetic acid, methanol, sodium acetate, sodium chloride, PBS, potassium nitrate, deionized water, physiological saline, citrate, citric acid, lactic acid, formic acid, TFA, nitric acid, phosphoric acid, hexafluoroisopropanol, or any combination thereof. In aspects, the adhesive composition comprises acetic acid at a concentration between 0.5% (w/w) and 10% (w/w). In other aspects, the adhesive composition does not include a solvent. In some embodiments, the adhesive composition comprises an additive such as glycerin, sorbitol, PEG, a coloring agent, cellulose derivatives, alginate, gelatin, pectin, gellan gum, starch, xanthan gum, cationic guar gum, agar, polysaccharides, vinyl polymers, PVA, acrylic resins, paraffin, a therapeutic agent, carboxyvinyl polymer, isinglass, a foaming agent, glutathione, heparin, or any combination thereof. When additives are included, the one or more peptides and/or one or more polymers described herein are included in a content that maintains adhesive activity. In other aspects, the adhesive composition does not comprise an additive. Those skilled in the art may choose an appropriate formulation, solvent, and/or additive according to the desired application. Applications of the adhesive composition include a dental adhesive, a surgical adhesive, an underwater adhesive, a subterranean adhesive, a marine adhesive, or a combination thereof. The concentration of the peptide and/or polymer in these formulations can vary widely, and will be selected primarily based on substrate compositions, fluid volumes, viscosities, and the like, in accordance with the particular mode of administration selected, the materials to be adhered, and adhesion strength required. In certain embodiments, the concentration of a peptide and/or polymer in a solution formulation for injection will range from 0.1% (w/w) to 15% (w/w) or about 0.1% (w/w) to about 15% (w/w).

The present invention further provides a method of adhering a first substrate to a second substrate. The method includes disposing the one or more peptides and/or one or more polymers and/or adhesive composition between the first substrate and the second substrate. In aspects, the one or more peptides and/or one or more polymers and/or adhesive composition is disposed by topical contact with the first and/or second substrate via a spray, an injection, or the like. The first substrate and/or second substrate may be a bone, a tooth, a tissue, a cell, glass, a metal, ceramic, rock, mineral, cement, concrete, wood, plastic, polymer, paper, cardboard, or any combination thereof. In certain aspects, where the first substrate comprises glass, metal, ceramic, wood, plastic, polymer paper, or cardboard, the second substrate comprises a cell, a tissue, or a combination thereof. In other certain aspects of the method, where the first substrate comprises bone or a tooth, the second substrate comprises a metal, a ceramic, or a combination thereof.

The present invention further provides a method for making the inventive polymers disclosed herein. In aspects of the method for making the inventive polymer, the at least one peptide is capped at the terminal end with any suitable polymerizable monomer. In aspects, the polymerizable monomer may comprise an ethylenically unsaturated monomer. In aspects, the polymerizable monomer may comprise an olefin-based functional group, a norbornene-amide hexanoic acid, (meth)acrylate, or a norbornene dicarboxyamide. The polymerizable monomers may be polymerized by ROMP, RAFT, or ATRP, aspects of which are further described in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51 (9), 1861-1881, which are hereby incorporated by reference.

After polymerization the inventive polymers may be characterized using any suitable technique(s). Typically, the inventive polymers are characterized by size-exclusion chromatography with multiangle light scattering (SEC-MALS), sometimes referred to as gel permeation chromatography (GPC), to ascertain degree of polymerization (DP) and molecular weight distribution (dispersity or Mw/Mn). Alternatively, or in addition to, the inventive polymers may be characterized by SDS-PAGE to ascertain degree of polymerization (DP) and molecular weight. Preferably, there is suitable agreement between the obtained DP and the theoretical DP based on the initial monomer-to-initiator ratio ([M]₀/[I]₀).

The present invention further provides methods for using the inventive polymers disclosed herein. In aspects, the inventive polymers may be used as a therapeutic, an antibody mimetic, a PPI disrupting agent, or any combination thereof. In some embodiments, the methods described herein can be used to treat or manage a condition of the eye, such as macular degeneration. In some embodiments, the methods described herein can be used to treat or manage a tumor, such as cancer. The method includes administering a therapeutically effective amount of a polymer described herein and a pharmaceutically acceptable excipient to a subject, a cell, or a tissue in need thereof. For example, the methods can include administering the polymer to provide a dose of from 10 ng/kg to 50 mg/kg to the subject. For example, the polymer dose can range from 5 mg/kg to 50 mg/kg, from 10 μg/kg to 5 mg/kg, or from 100 μg/kg to 1 mg/kg. The polymer dose can also lie outside of these ranges, depending on the particular polymer as well as the type of disease being treated. Frequency of administration can range from a single dose to multiple doses per week, or more frequently. In some embodiments, the polymer is administered from about once per month to about five times per week. In some embodiments, the polymer is administered once per week.

The polymer can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the polymer is administered intravenously, subcutaneously, intramuscularly, topically, orally, or a combination thereof. The methods described herein can comprise contacting a target tissue of the subject with the polymer or a metabolite or product thereof, contacting a target cell of the subject with the polymer or a metabolite or product thereof, and/or contacting a target receptor of the subject with the polymer or a metabolite or product thereof, and/or contacting a target peptide of the subject with the polymer or a metabolite or product thereof. In embodiments, the polymers described herein pass through the cell membrane and contact an intracellular target. Without wishing to be bound by any particular theory, it is believed that the PLP structure and charge described herein play an integral role in providing cell permeability.

### Aspects of the Invention

Various aspects are contemplated herein, several of which are set forth in the paragraphs below. It is explicitly contemplated that any aspect or portion thereof can be combined to form an aspect. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even through “less than Y” in isolation does not include Y.

Aspect A1: A polymer characterized by a formula (FX1):

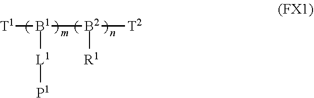

wherein:
each P¹ independently comprises a peptide;
at least one P¹, optionally all P¹, comprises: (i) a sequence having at least 75% or greater (e.g., 75% or greater, 80% or greater, 85% or greater, 90% or greater, 95% or greater, or 99% or greater) sequence identity of a tandem repeat peptide sequence found in a natural protein or natural peptide and/or (ii) at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues), and optionally in some embodiments, at least one P¹, and optionally all P¹, comprises sequence identity of a tandem repeat peptide sequence found in a natural protein or natural peptide and/or (ii) at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues);
T¹ and T² are each independently polymer backbone terminating groups that can be the same or different;
B¹ and B² are each independently polymer backbone subunits;
each L¹ is independently a linking group;
each R¹ is independently a substituent;
m is an integer selected from the range of 2 to 1000 (e.g., 2 to 500, 2 to 250, 2 to 100, 2 to 50, 2 to 30, 5 to 1000, 5 to 500, 5 to 250, 5 to 100, 5 to 50, 5 to 30, 20 to 500, 20 to 250, 20 to 100, 20 to 50, or 20 to 30);
n is an integer selected from the range of 0 to 1000 (e.g., 0 to 500, 0 to 250, 0 to 100, 0 to 50, 0 to 30, 2 to 1000, 2 to 500, 2 to 250, 2 to 100, 2 to 50, 2 to 30, 5 to 1000, 5 to 500, 5 to 250, 5 to 100, 5 to 50, 5 to 30, 20 to 500, 20 to 250, 20 to 100, 20 to 50, or 20 to 30);
each connecting line in formula (FX1) represents a covalent linkage comprising at least one of a single bond, a double bond, one or more atoms, or any combination thereof, optionally, for example, each connecting line represents a single bond or double bond;
each instance of B¹, B², L¹, R¹, and P¹ is the same as or different from any other instance of B¹, B², L¹, R¹, and P¹, respectively; and
when n is an integer from 1 to 1000 and/or at least one instance of P¹, optionally all P¹, is different from another instance of P¹, the polymer is a block copolymer or a statistical copolymer.

Aspect A2: The polymer of aspect A1, wherein the at least one P¹, optionally all P¹, comprises the sequence having at least 75% or greater (e.g., 75% or greater, 80% or greater, 85% or greater, 90% or greater, 95% or greater, or 99% or greater) sequence identity of a tandem repeat peptide sequence found in the natural protein or natural peptide or a modification thereof having at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues), and optionally in some embodiments, at least one P¹, and optionally all P¹, comprises the sequence identity of a tandem repeat peptide sequence found in the natural protein or natural peptide or a modification thereof having at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues).

Aspect A3: The polymer of aspect A1 or aspect A2, wherein the at least one P¹, optionally all P¹, comprises at least five amino acids (e.g., at least any of the following: 5, 7, 8, 10, 12, 15, 20, 25, or 30, optionally at least any of the following: 35, 40, 45, 50, 55, 60, 65, 70, 75, 80, 85, or 90, optionally, at less than any of the following: 100, 150, 200, 250, 300, 350, 400, 450, or 500).

Aspect A4: The polymer of any one of aspects A1-A3, wherein:
the at least one P¹, optionally all P¹, comprises a sequence having 75% or greater (e.g., 75% or greater, 80% or greater, 85% or greater, 90% or greater, 95% or greater, or 99% or greater) sequence identity of a tandem repeat peptide sequence found in a natural protein or natural peptide, and optionally in some embodiments, at least one P¹, and optionally all P¹, comprises the sequence identity of a tandem repeat peptide sequence found in a natural protein or natural peptide; and
the tandem repeat peptide sequence is found in a mussel or a secretion thereof, a spider silk, a silk protein, an ankyrin, a collagen, a keratin, an elastin, a resilin, a squid ring teeth protein, a fibroin, or an abductin.

Aspect A5: The polymer of any one of aspects A1-A4, wherein the tandem repeat peptide sequence is found in mussel foot protein.

Aspect A6: The polymer of any one of aspects A1-A5, wherein the tandem repeat peptide sequence comprises or consists of the following ten amino acids:
(1) alanine, (2) lysine, (3) proline or 4-hydroxyproline, (4) serine, (5) tyrosine or 3,4-dihydroxyphenylalanine, (6) 4-hydroxyproline or proline, (7) 4-hydroxyproline or proline, (8) threonine, (9) 3,4-dihydroxyphenylalanine or tyrosine, and (10) lysine.

Aspect A7: The polymer of any one of aspects A1-A6, wherein at least one P¹, optionally all P¹, comprises a sequence having 60% or greater (e.g., 60% or greater, 65% or greater, 70% or greater, 75% or greater, 80% or greater, 85% or greater, 90% or greater, 95% or greater, 99% or greater, or 100%) sequence identity of the tandem repeat peptide sequence.

Aspect A8: The polymer of any one of aspects A1-A7, wherein at least one P¹, optionally all P¹, comprises a sequence having 80% or greater (e.g., 80% or greater, 85% or greater, 90% or greater, 95% or greater, or 99% or greater) sequence identity of the tandem repeat peptide sequence and optionally in some embodiments, at least one P¹, and optionally all P¹, comprises the sequence identity of the tandem repeat peptide sequence.

Aspect A9: The polymer of any one of aspects A1-A8, wherein at least one P¹, optionally all P¹, comprises a sequence having 60% (e.g., 60% or greater, 65% or greater, 70% or greater, 75% or greater, 80% or greater, 85% or greater, 90% or greater, 95% or greater, 99% or greater, or 100%) or greater sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine.

Aspect A10: The polymer of any one of aspects A1-A9, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine, and optionally in some embodiments, at least one P¹, and optionally all P¹, comprises the sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine.

Aspect A11: The polymer of any one of aspects A1-A10, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 16 (AKPSYPPTYK), and optionally in some embodiments, at least one P¹, and optionally all P¹, comprises SEQ ID NO: 16 (AKPSYPPTYK).

Aspect A12: The polymer of any one of aspects A1-A11, wherein:
each amino acid in at least one P¹ independently is in a D or L configuration; and
optionally at least one P¹, optionally all P¹, comprises all D amino acids or all L amino acids.

Aspect A13: The polymer of any one of aspects A1-A12, wherein the at least one catechol residue comprises 3,4-dihydroxyphenylalanine.

Aspect A14: The polymer of any one of aspects A1-A13, wherein the at least one P¹, optionally all P¹, further comprises at least one lysine residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 lysine residues).

Aspect A15: The polymer of any one of aspects A1-A14, wherein m is an integer from 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30), n is 0, and at least 50% of all instances of P¹, optionally all instances of P¹, on a number basis comprise a sequence having 75% or greater (e.g., 75% or greater, 80% or greater, 85% or greater, 90% or greater, 95% or greater, or 99% greater) sequence identity of a tandem repeat peptide sequence found in a natural protein or natural peptide and optionally in some embodiments, at least one P¹, and optionally all P¹, comprises the sequence identity of a tandem repeat peptide sequence found in a natural protein or natural peptide.

Aspect A16: The polymer of any one of aspects A1-A15, wherein m is an integer from 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30), n is 0, and each instance of P¹ is the same and comprises a sequence having 75% or greater sequence identity of a tandem repeat peptide sequence found in a natural protein or natural peptide, and optionally in some embodiments, and optionally all P¹ comprises the sequence having 75% or greater sequence identity of a tandem repeat peptide sequence found in a natural protein or natural peptide.

Aspect A17: The polymer of any one of aspects A1-A16, wherein the at least one P¹, optionally all P¹, comprises at least 33 amino acid residues (e.g., at least any of the following: 33, 34, 35, 36, 37, 38, 39, 40, 45, 50, 55, 60, 65, 70, 75, 80, 85, or 90 amino acid residues, optionally less than 200, 150, or 100 amino acid residues).

Aspect A18: The polymer of any one of aspects A1-A17, wherein the at least one P¹, optionally all P¹, comprises a helix-loop-helix motif.

Aspect A19: The polymer of any one of aspects A1-A18, wherein the tandem repeat peptide sequence is found in an ankyrin protein.

Aspect A20: The polymer of any one of aspects A1-A19, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 2 (DKXGRTPLHLAARXGHLEVVKLLLXXGADVNAK), wherein each X independently is a hydrophilic amino acid residue, and optionally in some embodiments, at least one P¹, and optionally all P¹, comprises SEQ ID NO: 2 (DKXGRTPLHLAARXGHLEVVKLLLXXGADVNAK), wherein each X independently is a hydrophilic amino acid residue.

Aspect A21: The polymer of any one of aspects A1-A20, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 3 (DKNGRTPLHLAARNGHLEVVKLLLEAGADVNAK), and optionally in some embodiments, at least one P¹, and optionally all P¹, comprises SEQ ID NO: 3 (DKNGRTPLHLAARNGHLEVVKLLLEAGADVNAK).

Aspect A22: The polymer of any one of aspects A1-A21, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 4 (DXXGXTPLHLAAXXGHLEIVEVLLXAGADVNAX), wherein each X independently is a hydrophilic amino acid residue, and optionally in some embodiments, at least one P¹, and optionally all P¹, comprises SEQ ID NO: 4 (DXXGXTPLHLAAXXGHLEIVEVLLXAGADVNAX), wherein each X independently is a hydrophilic amino acid residue.

Aspect A23: The polymer of any one of aspects A1-A22, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 5 (DKNGRTPLHLAARNGHLEIVEVLLEAGADVNAK), and optionally in some embodiments, at least one P¹, and optionally all P¹, comprises SEQ ID NO: 5 (DKNGRTPLHLAARNGHLEIVEVLLEAGADVNAK).

Aspect A24: The polymer of any one of aspects A1-A23, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 6 (DAAREGFLDTLVVLHRAGAR), and optionally in some embodiments, at least one P¹, and optionally all P¹, comprises SEQ ID NO: 6 (DAAREGFLDTLVVLHRAGAR).

Aspect A25: The polymer of any one of aspects A1-A24, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 7 (FLDTLVVLHR), and optionally in some embodiments, at least one P¹, and optionally all P¹, comprises SEQ ID NO: 7 (FLDTLVVLHR).

Aspect A26: The polymer of any one of aspects A1-A25, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 8 (ALPNAPNSYGRRPIQVMMMGSARVAELLLLHGAE), and optionally in some embodiments, at least one P¹, and optionally all P¹, comprises SEQ ID NO: 8 (ALPNAPNSYGRRPIQVMMMGSARVAELLLLHGAE).

Aspect A27: The polymer of any one of aspects A1-A26, wherein at least one P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 9 (PNCADPATLTRPVHDAAREGFLDTLVVLHRA), and optionally in some embodiments, at least one P¹, and optionally all P¹, comprises SEQ ID NO: 9 (PNCADPATLTRPVHDAAREGFLDTLVVLHRA).

Aspect A28: The polymer of any one of aspects A1-A27, wherein the sequence having 75% or greater sequence identity of SEQ ID NO: 9 has a point mutation or substitution to comprise a methionine.

Aspect A29: The polymer of aspect A28, or any preceding aspect, wherein P¹, optionally all P¹, comprises a sequence having 75% or greater (e.g., 75% or greater, 80% or greater, 85% or greater, 90% or greater, 95% or greater, or 99% or greater) sequence identity of SEQ ID NO: 15 (PNMADPATLTRPVHDAAREGFLDTLVVLHRA), and optionally in some embodiments, at least one P¹, and optionally all P¹, comprises SEQ ID NO: 15 (PNMADPATLTRPVHDAAREGFLDTLVVLHRA).

Aspect A30: The polymer of any one of aspects A1-A29, wherein at least one P¹, optionally all P¹, comprises a sequence having 75% or greater (e.g., 75% or greater, 80% or greater, 85% or greater, 90% or greater, 95% or greater, 99% or greater, or 100%) sequence identity of SEQ ID NO: 10 (GSGSGS), and optionally in some embodiments, at least one P¹, and optionally all P¹, comprises SEQ ID NO: 10 (GSGSGS).

Aspect A31: The polymer of any one of aspects A1-A30, wherein at least one P¹, optionally all P¹, comprises a sequence having 75% or greater (e.g., 75% or greater, 80% or greater, 85% or greater, 90% or greater, 95% or greater, 99% or greater, or 100%) sequence identity of SEQ ID NO: 11 (KIABAVBLBAEE), and optionally in some embodiments, at least one P¹, and optionally all P¹, comprises SEQ ID NO: 11 (KIABAVBLBAEE).

Aspect A32: The polymer of any one of aspects A1-A31, wherein at least one P¹, optionally all P¹, comprises a sequence having 75% or greater (e.g., 75% or greater, 80% or greater, 85% or greater, 90% or greater, 95% or greater, 99% or greater, or 100%) sequence identity of SEQ ID NO: 12 (SWTWENGKWTWK), and optionally in some embodiments, at least one P¹, and optionally all P¹, comprises SEQ ID NO: 12 (SWTWENGKWTWK).

Aspect A33: The polymer of any one of aspects A1-A32, wherein at least one P¹ comprises a sequence having 75% or greater (e.g., 75% or greater, 80% or greater, 85% or greater, 90% or greater, 95% or greater, 99% or greater, or 100%) sequence identity of SEQ ID NO: 13 (GSGSGRGSGSGE), and optionally in some embodiments, at least one P¹, and optionally all P¹, comprises SEQ ID NO: 13 (GSGSGRGSGSGE).

Aspect A34: The polymer of any one of aspect A1-A33, wherein at least one P¹, optionally all P¹, comprises a sequence having 75% or greater (e.g., 75% or greater, 80% or greater, 85% or greater, 90% or greater, 95% or greater, 99% or greater, or 100%) sequence identity of SEQ ID NO: 14 (NGRTPLHLAARNGHLEVVKLLLEAGADVNAKDK), and optionally in some embodiments, at least one P¹, and optionally all P¹, comprises SEQ ID NO: 14 (NGRTPLHLAARNGHLEVVKLLLEAGADVNAKDK).

Aspect A35: The polymer of any one of aspects A1-A34, wherein the polymer comprises at least one of the following properties:
each P¹ independently, optionally all P¹, comprises from 5-100 amino acid residues;
m is an integer from 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30);
n is an integer from 0 to 100 (e.g., 0 to 100, 0 to 90, 0 to 80, 0 to 70, 0 to 60, 0 to 50, 0 to 40, 0 to 30, 0 to 20, 0 to 10, 1 to 100, 1 to 90, 1 to 80, 1 to 70, 1 to 60, 1 to 50, 1 to 40, 1 to 30, 1 to 20, 1 to 10, 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30);
m is an integer from 2 to 100 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30), n is 0, and at least one instance of P¹, optionally all P¹, is different from another instance of P¹;
the degree of polymerization of polymer backbone subunits is an integer from 2 to 200 (e.g., 2 to 100, 2 to 90, 2 to 80, 2 to 70, 2 to 60, 2 to 50, 2 to 40, 2 to 30, 2 to 20, 2 to 10, 4 to 100, 4 to 90, 4 to 80, 4 to 70, 4 to 60, 4 to 50, 4 to 40, 4 to 30, 4 to 20, 4 to 10, 10 to 50, 10 to 40, 10 to 30, 20 to 50, 20 to 40, or 20 to 30), or 2 to 50;
a molecular weight of from 1 kDa to 1,000 kDa (e.g., from 1 kDa to 1000 kDa, from 1 kDa to 500 kDa, from 1 kDa to 100 kDa, from 1 kDa to 60 kDa, from 1 kDa to 50 kDa, from 1 kDa to 70 kDa, 5 kDa to 2,000 kDa, from 5 kDa to 1,000 kDa, from 5 kDa to 500 kDa, from 5 kDa to 100 kDa, from 5 kDa to 70 kDa, from 5 kDa to 60 kDa, from 5 kDa to 50 kDa, from 10 kDa to 1,000 kDa, from 10 kDa to 500 kDa, from 10 kDa to 100 kDa, from 10 kDa to 70 kDa, from 10 kDa to 60 kDa, or from 10 kDa to 50 kDa);
a peptide density of at least 50% (e.g., at least 50%, at least 55%, at least 60%, at least 65%, at least 70%, at least 75%, at least 80%, at least 85%, at least 90%, at least 95%, at least 99%, or 100%), as defined by equation m/(m+n)×100;
a combination thereof; or
any combination thereof.

Aspect A36: The polymer of any one of aspects A1-A35, wherein at least one P¹, optionally all P¹, comprises a sequence comprising gaps between one or more amino acid residues, wherein the gaps comprise up to five amino acid residues or other spacer molecules.

Aspect A37: The polymer of any one of aspects A1-A36, wherein the polymer is prepared by a living polymerization method optionally selected from ring-opening metathesis polymerization (ROMP), reversible addition-fragmentation chain transfer polymerization (RAFT), or atom transfer radical polymerization (ATRP).

Aspect A38: The polymer of any one of aspects A1-A37, wherein at least one P′, optionally all P¹, is characterized by a substructure (S3):

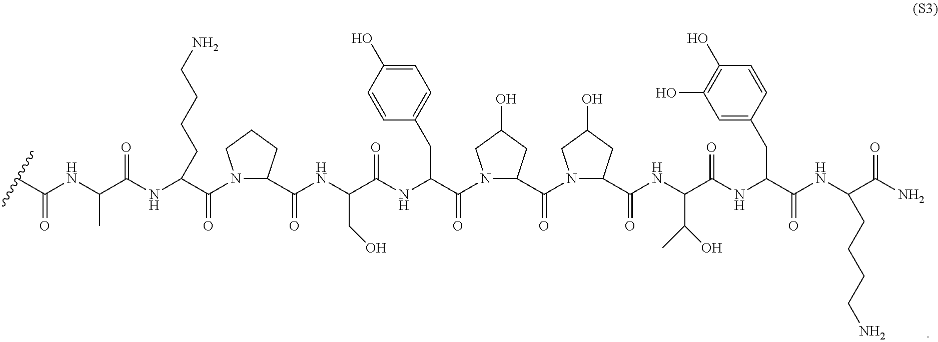 [SMILES: *C(=O)NC(C)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)c(O)c1)C(=O)NC(CCCCN)C(N)=O)C(C)O]

Aspect A39: The polymer of any one of aspects A1-A38, wherein each of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl.

Aspect A40: The polymer of any one of aspects A1-A39, wherein the polymer of formula (FX1) is a polymer characterized by a formula (FX2):

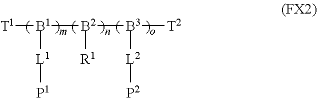

wherein:
each L² is a linking group;
each B³ is a polymer backbone subunit;
o is an integer from 0 to 1000 (e.g., from 1 to 1000, from 1 to 500, from 1 to 100, from 1 to 60, from 1 to 50, from 1 to 70, 5 to 2,000, from 5 to 1,000, from 5 to 500, from 5 to 100, from 5 to 70, from 5 to 60, from 5 to 50, from 10 to 1,000, from 10 to 500, from 10 to 100, from 10 to 70, from 10 to 60, or from 10 to 50);
each instance of P¹ is the same;
each P² is a peptide;
each instance of P² is the same or different, and each P² is different from P¹;
optionally at least one P², optionally all P², comprises: (i) a sequence having at least 75% or greater (e.g., 75% or greater, 80% or greater, 85% or greater, 90% or greater, 95% or greater, or 99% or greater) sequence identity of a tandem repeat peptide sequence found in a natural protein or natural peptide and/or (ii) at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues), and optionally in some embodiments, at least one P², and optionally all P², comprises the sequence identity of a tandem repeat peptide sequence found in a natural protein or natural peptide and/or at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues);
each instance of B¹, B², B³, L¹, L², and R¹ is the same as or different from any other instance of B¹, B², B³, L¹, L², and R¹, respectively;
when n and/or o is an integer from 1 to 1000 (e.g., from 1 to 1000, from 1 to 500, from 1 to 100, from 1 to 60, from 1 to 50, from 1 to 70, 5 to 2,000, from 5 to 1,000, from 5 to 500, from 5 to 100, from 5 to 70, from 5 to 60, from 5 to 50, from 10 to 1,000, from 10 to 500, from 10 to 100, from 10 to 70, from 10 to 60, or from 10 to 50), the polymer is a block copolymer or a statistical copolymer; and
at least one of P¹, optionally all P¹, and/or at least one of P², optionally all P², independently comprise a sequence having at least 75% or greater (e.g., 75% or greater, 80% or greater, 85% or greater, 90% or greater, 95% or greater, or 99% or greater) sequence identity of any one of SEQ ID NO: 1-16, and optionally in some embodiments, at least one P², and optionally all P², comprises the sequence identity of any one of SEQ ID NO: 1-16.

Aspect A41: The polymer of aspect A40, or any preceding aspect, wherein at least one P², optionally all P², comprises the sequence having at least 75% or greater (e.g., 75% or greater, 80% or greater, 85% or greater, 90% or greater, 95% or greater, or 99% or greater) sequence identity of a tandem repeat peptide sequence found in the natural protein or natural peptide or a modification thereof having at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues), and optionally in some embodiments, at least one P², and optionally all P², comprises the sequence identity of a tandem repeat peptide sequence found in the natural protein or natural peptide or a modification thereof having at least one catechol residue (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, or 100 catechol residues; optionally less than 200, 150, 125, or 100 catechol residues).

Aspect A42: The polymer of any one of aspects A1-A41, wherein each instance of L¹ and L² independently comprises a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, wherein each L¹ is configured with one or more suitable functional groups to covalently attach B¹ with P¹, and wherein each L² is configured with one or more suitable functional groups to covalently attach B³ with P².

Aspect A43: The polymer of any one of aspects A1-A42, wherein at least one of B¹, B², or B³, optionally all of B¹, optionally all of B², optionally all of B³, or optionally all of B¹, B², and B³, comprises a polymerized monomer comprising an unsaturated monomer.

Aspect A44: The polymer of aspect A43, or any preceding aspect, wherein the unsaturated monomer comprises an ethylenically unsaturated monomer, a norbornene monomer, or a norbornene dicarboxyimide.

Aspect A45: The polymer of any one of aspects A1-A44, wherein each instance of a 5 substructure (Sla):

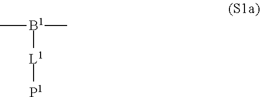

in formula (FX1) or (FX2) is independently characterized by a substructure (S1b), (S1c), (S1d), or (S1e):

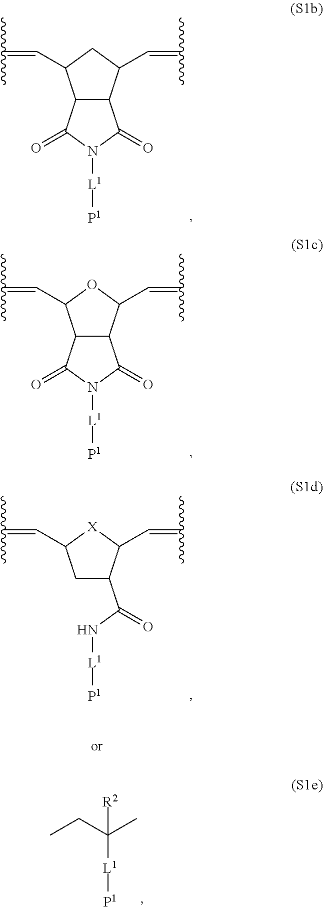

wherein L¹ is optionally present, R² is H or C₁-C₃ alkyl, and X is CH₂ or O.

Aspect A46: The polymer of any one of aspects A40-A45, or any preceding aspect, wherein each instance of a substructure (S2a):

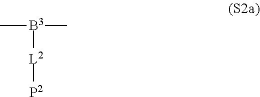

in formula (FX2) is independently characterized by a substructure (S2b), (S2c), (S2d), or (S2e):

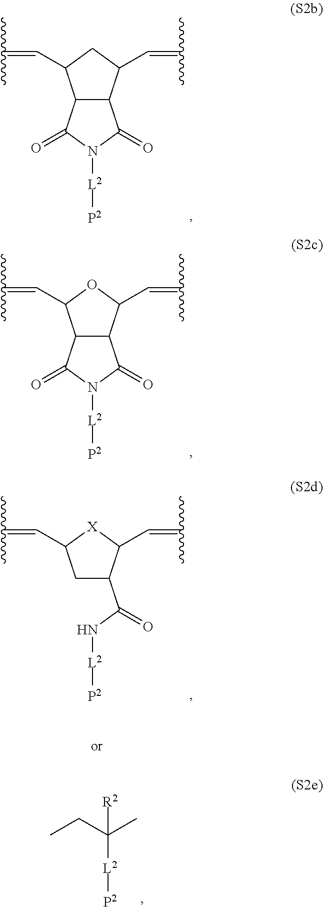

wherein L² is optionally present, R² is H or C₁-C₃ alkyl, and X is CH₂ or O.

Aspect A47: The polymer of any one of aspects A1-A46, wherein, when subjected to an atomic force microscopy adhesion test, the polymer has an adhesion force of at least 100 pN (e.g., at least 100 pN, at least 150 pN, at least 200 pN, at least 250 pN, at least 300 pN, at least 350 pN, at least 400 pN, at least 450, at least 500 pN, or at least 750 pN, optionally less than 2000 pN, 1500 pN, 1250 pN, or 1000 pN).

Aspect B1: An adhesive composition comprising the polymer of any one of aspects A1-A47.

Aspect B2: The adhesive composition of aspect B1, or any preceding aspect, wherein the adhesive composition is a dental adhesive, a surgical adhesive, an underwater adhesive, a subterranean adhesive, or a marine adhesive.

Aspect B3: The adhesive composition of aspect B1 or aspect B2, or any preceding aspect, further comprising a bone, a tooth, a tissue, a cell, glass, a metal, ceramic, rock, mineral, cement, concrete, wood, plastic, polymer, paper, cardboard, or any combination thereof.

Aspect C1: A method of adhering a first substrate to a second substrate, the method comprising:
disposing the polymer of any one of aspects A0-A47, or any preceding aspect, between the first substrate and the second substrate;
wherein the polymer optionally is in a form of an adhesive composition.

Aspect C2: The method of aspect C1, or any preceding aspect, wherein the adhesive composition comprises the adhesive composition of any one of aspects B¹-B³, or any preceding aspect.

Aspect C3: The method of aspect C1 or C₂, or any preceding aspect, wherein:
the first substrate comprises glass, metal, ceramic, wood, plastic, polymer paper, or cardboard, or a combination thereof; and
the second substrate comprises a cell, a tissue, or a combination thereof.

Aspect C4: The method of aspect C1 or C₂, or any preceding aspect, wherein:
the first substrate comprises bone or a tooth, or a combination thereof; and the second substrate comprises a metal, a ceramic, or a combination thereof.

Aspect C5: The method of aspect C1 or C₂, or any preceding aspect, wherein:
the first substrate and the second substrate independently comprise a bone, a tooth, a tissue, a cell, glass, a metal, ceramic, rock, mineral, cement, concrete, wood, plastic, polymer, paper, cardboard, or any combination thereof.

Aspect D1: A method of making the polymer of any one of aspects A1-A47, or any preceding aspect, the method comprising:
synthesizing at least one P¹;
capping the at least one P¹ peptide at a terminal end with a polymerizable monomer that, once polymerized, becomes polymer backbone subunit B¹, thereby forming a polymerizable P¹ monomer;
polymerizing the polymerizable P¹ monomer.

Aspect D2: The method of aspect D1, or any preceding aspect, wherein:
the synthesizing step comprises solid-phase synthesis using protected amino acids;
the polymerizable monomer comprises an ethylenically unsaturated monomer optionally comprising norbornene or a (meth)acrylate; and
the polymerizing step comprises ROMP, RAFT, or ATRP.

Aspect E1: A method comprising use of the polymer of any one of aspects A1-A47, or any preceding aspect, as a therapeutic, as an antibody mimetic, as a protein-protein interaction (PPI) disrupting agent, or any combination thereof.

Aspect E2: The method of aspect E1, or any preceding aspect, further comprising administering a therapeutically effective amount of the polymer and a pharmaceutically acceptable excipient to a subject, cell, or tissue in need thereof, optionally wherein the polymer is effective for treating macular degeneration, a tumor (e.g., cancer, such as breast cancer), or a symptom or manifestation thereof.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experiment error and deviation should be accounted for.

### Materials and Methods:

Peptide Synthesis: Peptides were synthesized on a Liberty Blue (CEM) Automated Microwave Peptide Synthesizer using standard solid-phase protocols and Fmoc-protected amino acids. Rink amide MBHA resin was used to give C-terminal amide. The peptides were capped with Norbornene-amino hexanoic acid as previously described in Blum et al., Peptides Displayed as High Density Brush Polymers Resist Proteolysis and Retain Bioactivity, J. Am. Chem. Soc. 2014, 136 (43), 15422-15437, which is hereby incorporated by reference. Cleavage from the resin was performed in 95:2.5:2.5 (% v/v) trifluoroacetic acid (TFA), triisopropyl silane, and water, respectively, for 2 h. Following evaporation of TFA, the cleaved peptides were precipitated in cold diethyl ether and dried under vacuum to yield solid crude.

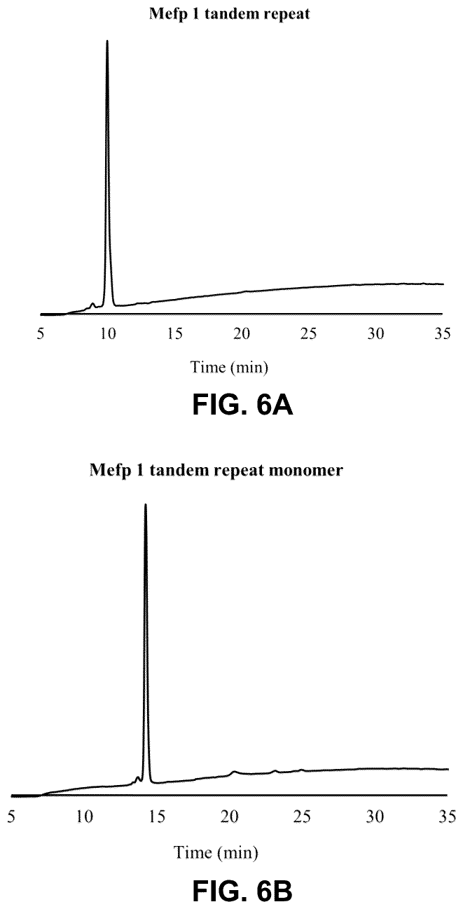

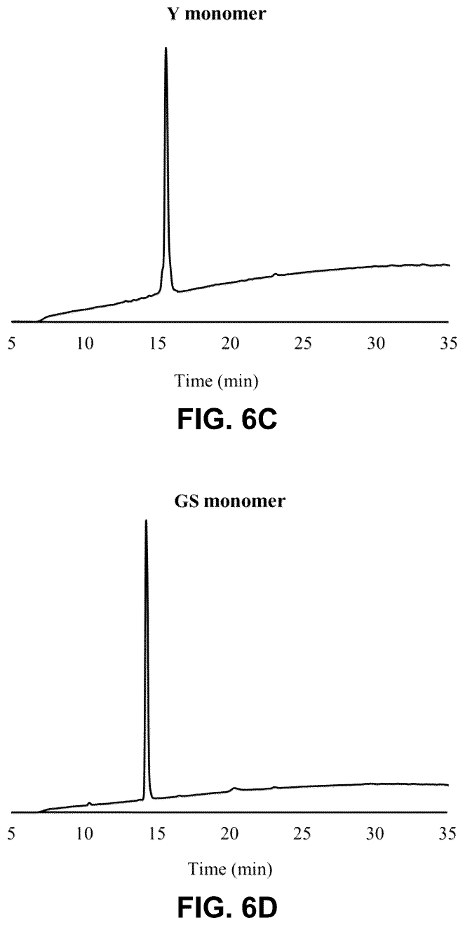

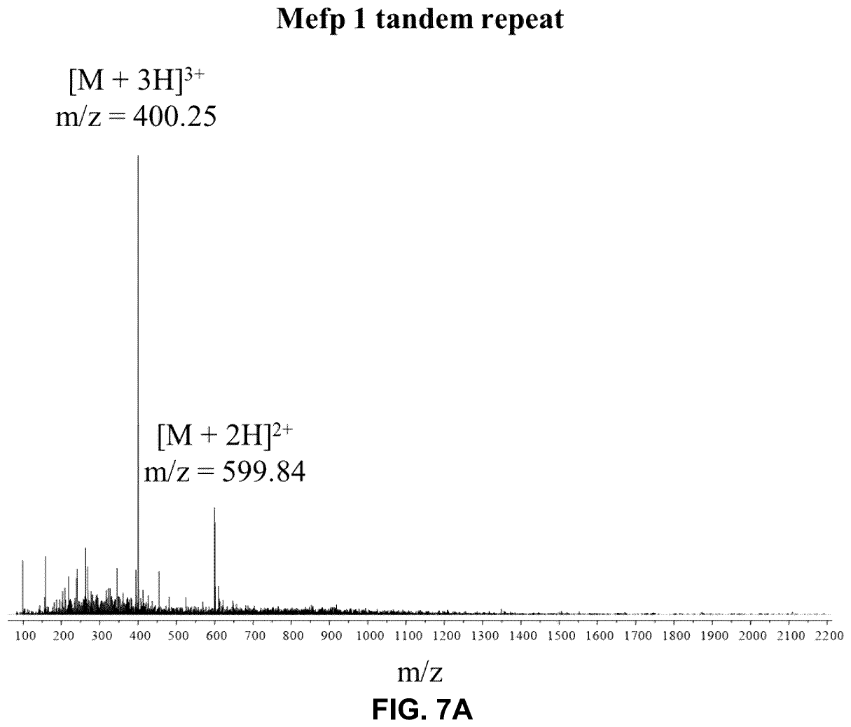

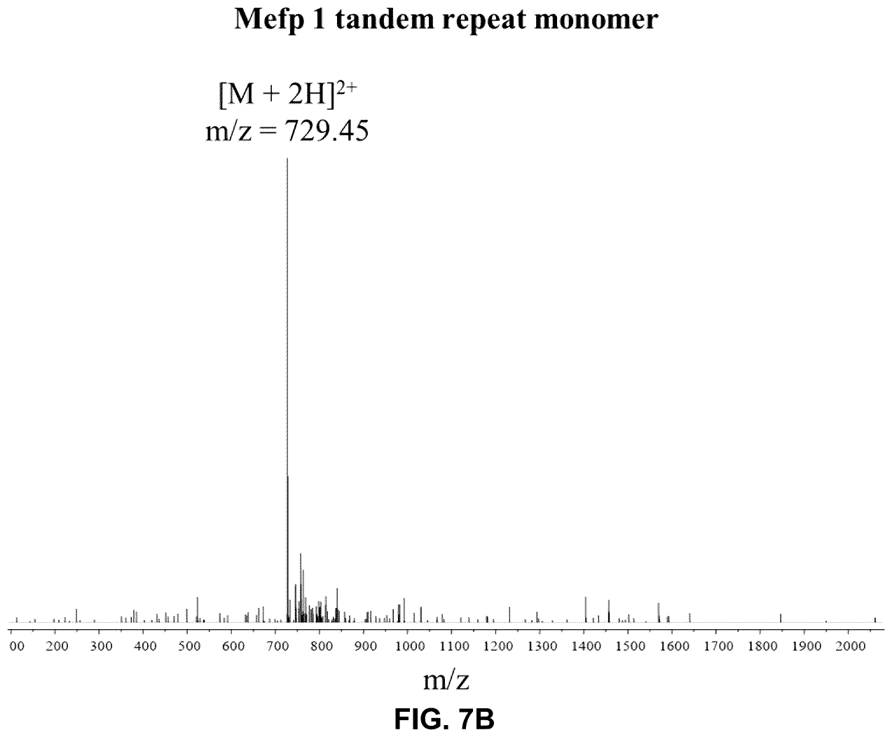

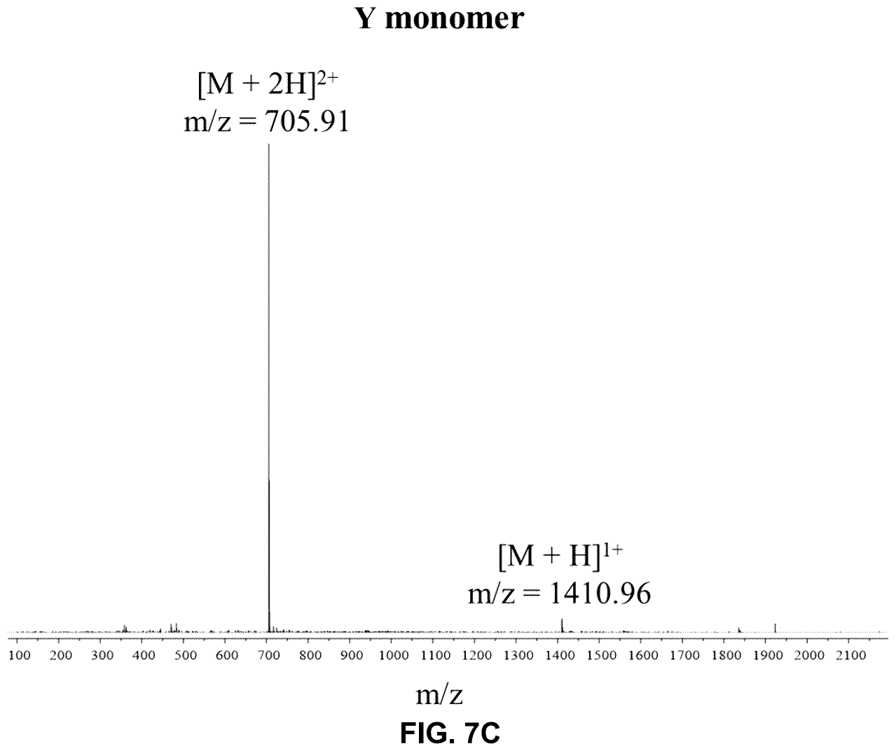

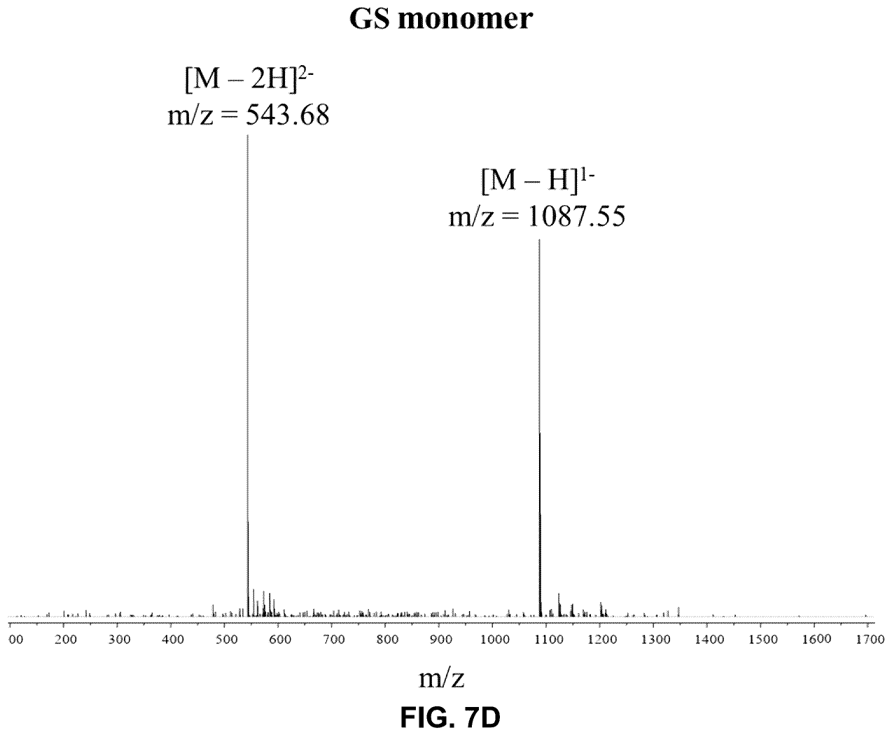

Peptides were purified with a Jupiter Proteo 90 Å Phenomenex column (2050×25.0 mm) on an Armen Glider CPC preparatory phase HPLC to yield 90-95% purity, confirmed by analytical HPLC (e.g., FIGS. 6A-6D). For all RP-HPLC purifications, gradient solvent systems utilized Buffer A (water with 0.1% TFA) and Buffer B (acetonitrile with 0.1% TFA). A gradient of 15-45% buffer B over 1 h was used to purify all peptides and monomers. Pure products were then analyzed by electrospray ionization mass spectroscopy (ESI-MS) on a Bruker amaZon SL to confirm molecular weight (e.g., FIGS. 7A-7D).

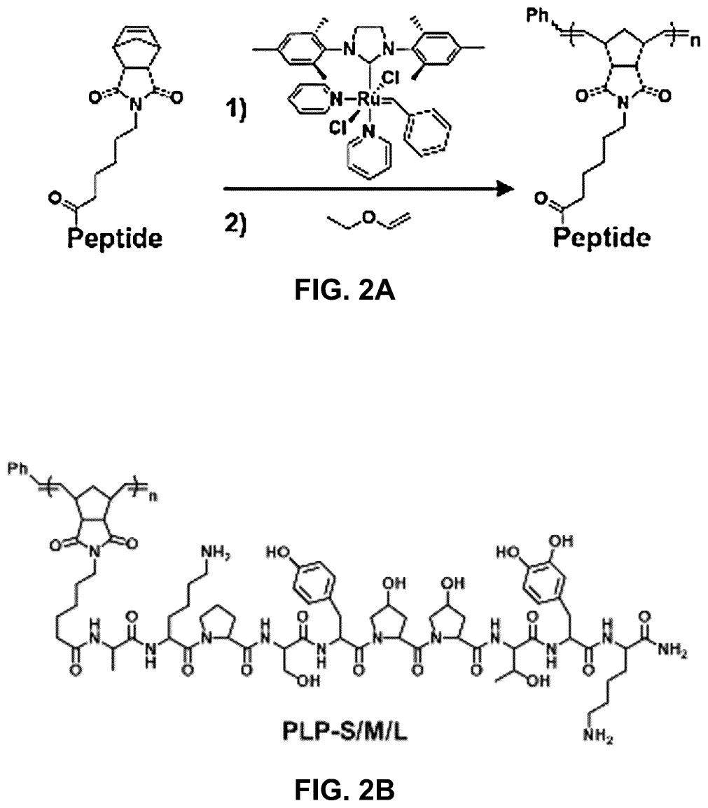

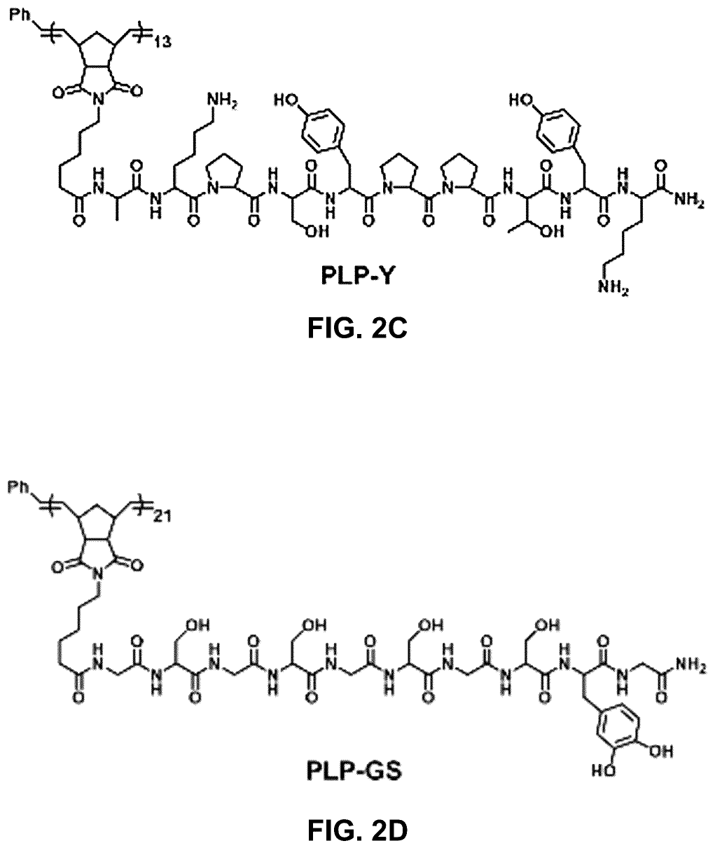

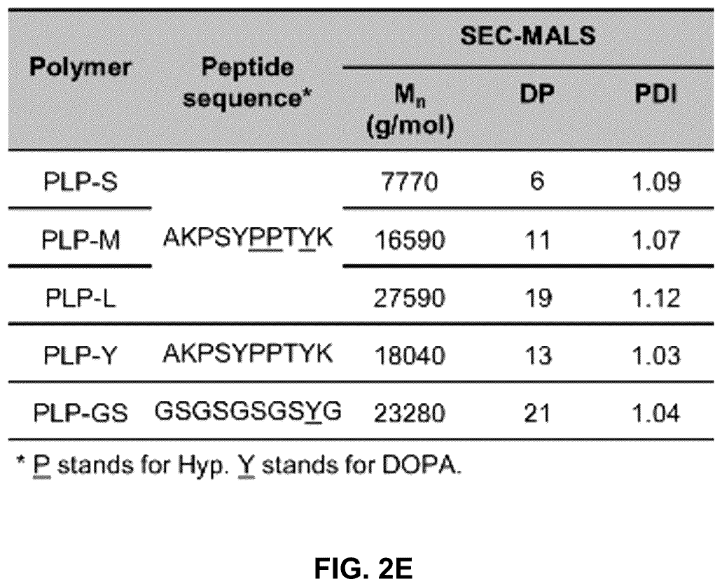

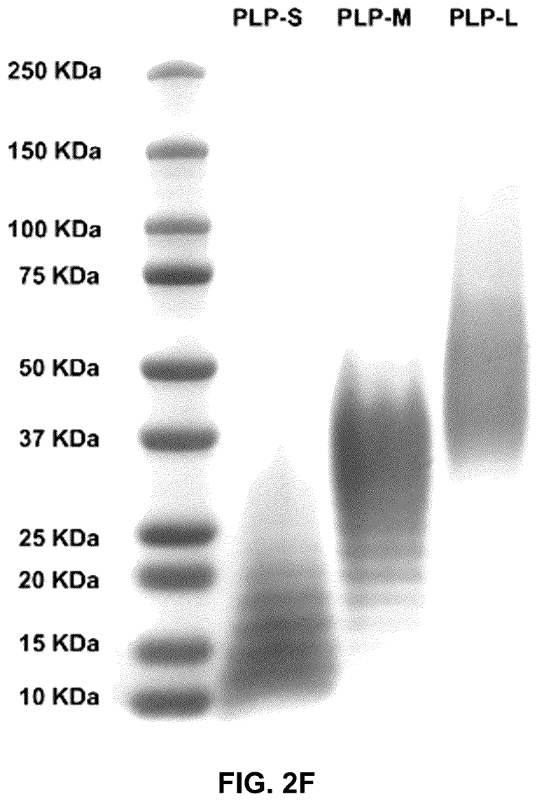

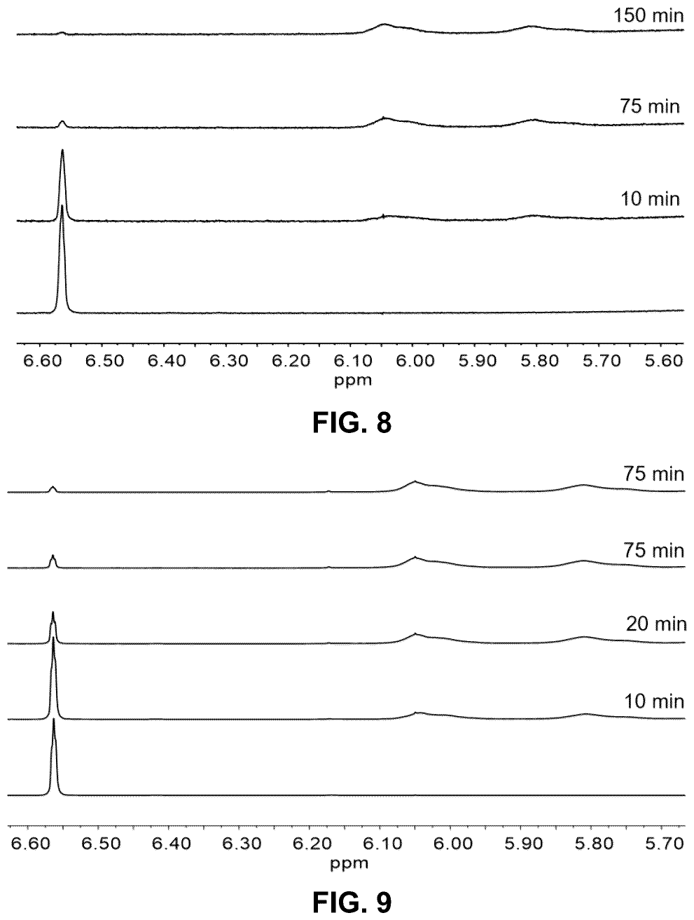

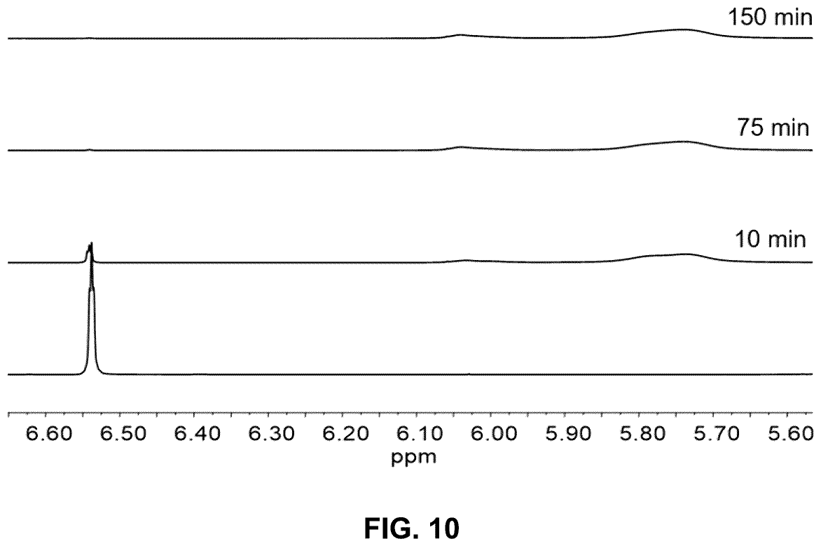

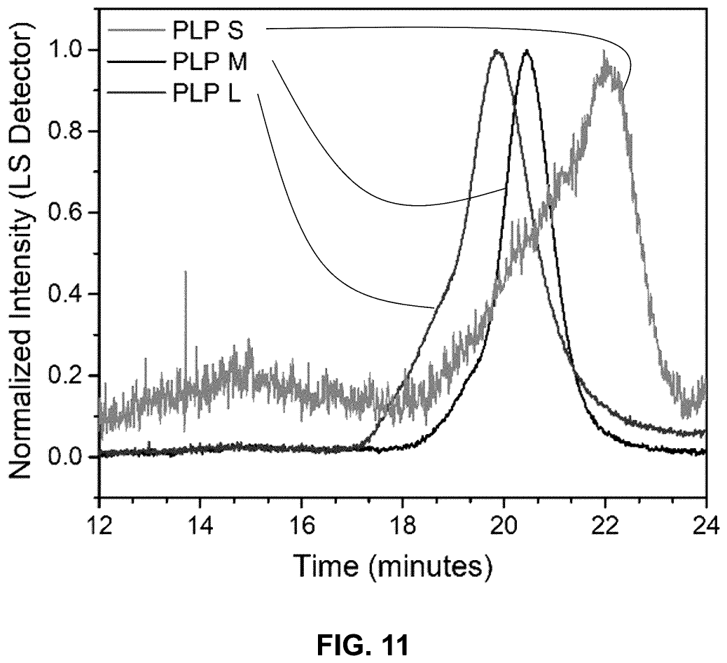

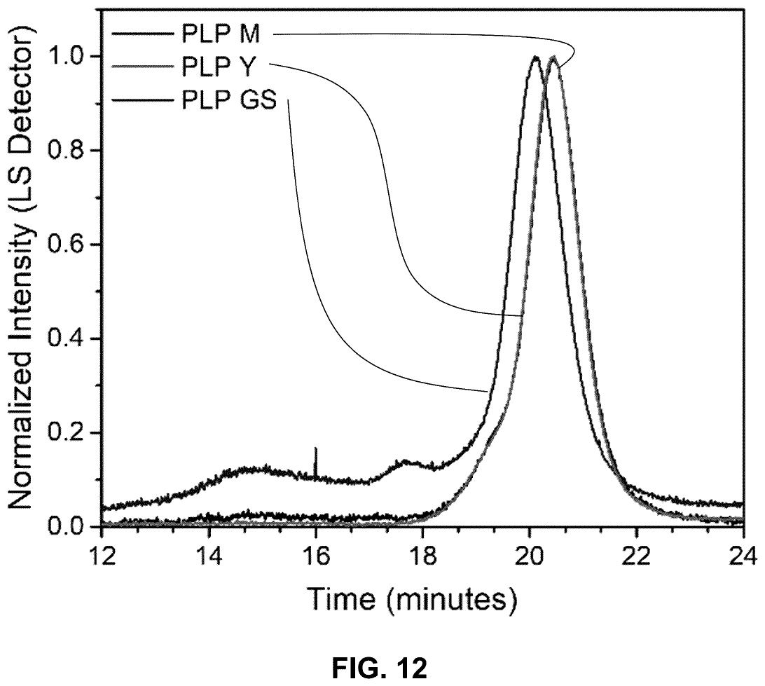

Polymerization: Protein-Like Polymers (“PLPs”) incorporating the purified peptides were synthesized following the general mechanism provided in FIG. 14B with the expected properties described in FIG. 14A, aspects of which are further described in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51 (9), 1861-1881, which are hereby incorporated by reference. Polymerization reactions were carried out in a glovebox under a nitrogen atmosphere as described in Blum et al. A typical protocol used to generate a polymer with DP=15 involved mixing the monomer (e.g., 15 mg, 0.0103 mmol, FIGS. 6A-6D and FIGS. 7A-7D) with the initiator (e.g., 0.5 mg, 0.00068 mmol, FIG. 2A) in dry DMF (0.5 mL) 1H NMR was used to confirm complete consumption of the monomer and to determine the time period required to reach completion (e.g., FIGS. 8-10). Typically about 150 min was required for complete conversion. The polymers were terminated with ethyl vinyl ether (10 eq) for 1 h at room temperature, precipitated and washed with cold diethyl ether three times and collected by centrifugation. Polymers molecular weight and polydispersity were determined by SEC MALS (Phenomenex Phenogel 5μ 103 Å, 1K-75K, 300×7.8 mm in series with a Phenomenex Phenogel 5μ 103 Å, 10K-100K, 300×7.8 mm) at 65° C. in 0.05M LiBr in DMF, using a ChromTech Series 1500 pump equipped with a multi-angle light scattering detector (DAWN-HELIOS II, Wyatt Technology) and a refractive index detector (Wyatt Optilab TrEX) normalized to a 30,000 MW polystyrene standard. (e.g., FIGS. 11-12).

AFM measurements: Silicon wafer chips (5×5 mm) (SPI supplies) were coated with one of the adhesive materials to a density of 200 μg cm⁻² by placing each chip in a 24-well plate and adding a 500 μL solution of the adhesive material. All solutions contained 1% acetic acid and 1 mg ml⁻¹ of the adhesive component. 500 μL of 0.1 M carbonate-bicarbonate buffer (pH=9.0) were added to each well to neutralize the acid which promotes immediate adherence to the surface. The solution was incubated for 10 min at room temperature and then aspirated. The wells were rinsed twice with pure water and the chips dried in open air prior to measurement.

All measurements were performed on a Bruker Dimension Icon AFM system. Coated chips were scanned at rate of 1 Hz with amplitude of 1.5 μm, the total loading force was ˜20 nN. The tip radius was 1 nm and the spring constant was 1 N/m. Five different locations were examined for each sample, and at each location ten measurement were performed along a linear line. Overall 50 measurement were taken for each sample. All experimental data were processed by NanoScope Analysis Software (Veeco Instrument Inc.).

Centrifugal Adhesion Measurement: The middle two rows of a 384-well plate (48 wells) were coated with the adhesive materials for centrifugal adhesion testing as described in Chen et al., High-Throughput Screening Test for Adhesion in Soft Materials Using Centrifugation, ACS Cent. Sci. 2021, which is hereby incorporated by reference. Briefly, the materials were dissolved in an aqueous solution of 1% acetic acid to a concentration of 5 mg ml-1. Mefp 1 protein was purchased from Sigma-Aldrich and diluted to the same concentration. Each well was pipetted with 20 μl of the corresponding material solution and then 20 μl of 0.1 M carbonate-bicarbonate buffer (pH=9.0) to neutralize the solution. The plate was centrifuged at 4700×g for 5 minutes to deposit the material at the bottom of the wells. A 20 μL silica microparticle suspension (radius=5 μm, Corpuscular Inc.) was pipetted into each well, and the plate was again centrifuged at 4700×g for 30 minutes to ensure good contact. Each well was then filled with water, followed by an absorbance measurement at a wavelength of 900 nm using a Synergy HTX Multi-Mode Reader (BioTek) as the 0×g reference. The plate was sealed underwater with the MicroAmp™ Optical Adhesive Film (Applied Biosystems™) and centrifuged with the well facing outward at various centrifugal speeds for 30 seconds. The plate was unsealed underwater to let detached particles fall into the water tank, and measured using the plate reader again. The plate was sealed again underwater and spun at a different centrifugal speed. Fractional absorbance was then calculated with the following equation:

$$Fractional Absorbance = \frac{{Absorbance}_{x \times g} - {Absorbance}_{blank}}{{Absorbance}_{0 \times g} - {Absorbance}_{blank}}$$

Cell immobilization: Wells of non-coated glass-bottom 24-well plates (MatTek) were treated with either the adhesive polymer or other materials to a density of 3.5 μg cm-1 by adding 300 μl of 1% acetic acid solution in water to each well. 65 μl of either polymer or protein solution of 1 mg ml-1 were added to separate wells. 300 μl of 0.1 M carbonate-bicarbonate buffer solution pH=9.0 were added to each well to neutralize the acid which promotes immediate adherence of the adhesive materials to the surface. The solution was incubated for 10 min in room temperature and then aspirated. The wells were rinsed twice with pure water.

HeLa cells (ATCC, CCL-2), aortic smooth muscle cells (ATCC, PCS-100-012), and E.G7 cells were grown to confluence before passaging and suspending in FBS-free media. Cells of each line were plated in either an untreated or a polymer- or protein-coated well in a concentration of 50,000 cells per well. The plate was incubated for 24 hours at 37° C. The wells were then rinsed 3 times with warm PBS and stained with a 20 mM Hoechst stain solution (Invitrogen), 1 drop per well.

The plates were imaged with a Lionheart FX automated microscope (BioTek) using a DAPI filter. Image processing and analysis were performed by Gen5 software.

### Example 1

This example provides an exemplary synthesis of a mussel adhesive-inspired proteomimetic polymer with promise to replicate function of complex proteins.

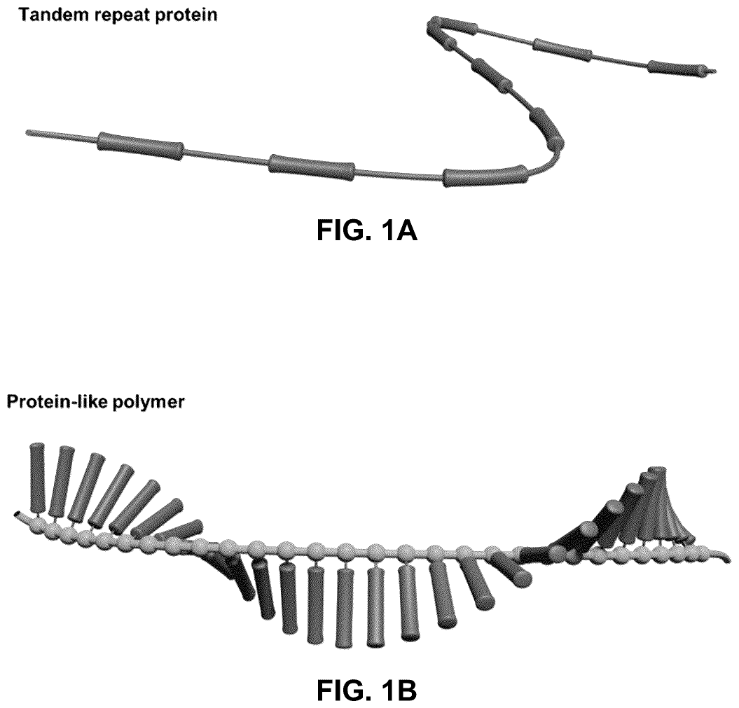

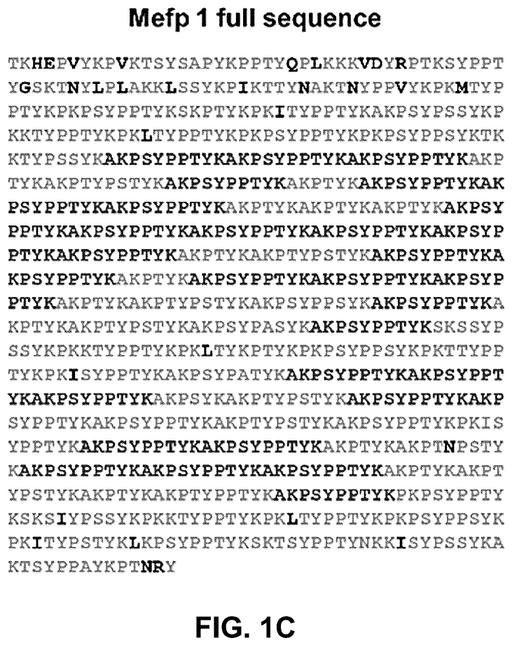

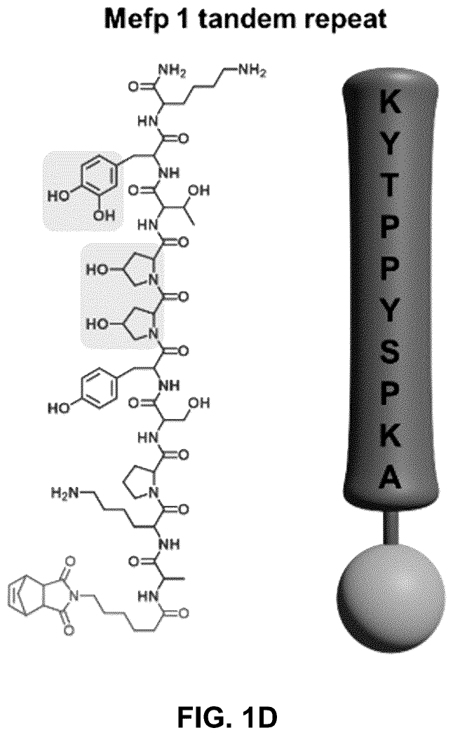

It was hypothesized that if the key repeat sequences were polymerized as brush polymers, the performance of TRPs could be recapitulated, in a proteomimetic manner (FIGS. 1A-1D). It was reasoned that the relatively short repeat sequences can be readily synthesized in the lab by solid-phase peptide synthesis (SPPS) and modified at their termini with polymerizable, olefin-based functional groups. Polymerization of these monomers would lead to densely functionalized brush polymers with peptide side chains as repeat units along the synthetic polymer backbone as opposed to being displayed in a linear fashion as in the natural protein (FIGS. 1A-1B). These proteomimetic macromolecules, or protein-like polymers (PLPs), exhibit a dense array of the repeat sequence as a multivalent brush. In this form it is believed that TRPs may have enhanced properties compared with their linear TRP natural counterparts in which some of the sequences are buried and are not surface accessible.

The tandem repeat peptide unit of the mussel foot protein was reconstituted (FIGS. 1C-1D). There are over eight different proteins that are secreted from the mussel foot which are responsible for the coating and adhesion of the mussel byssus. Most of which contain short tandem repeats in which one or more of the residues are 3,4-dihydroxyphenylalanine (L-DOPA). The most studied mussel adhesive protein is foot protein 1 of the genus Mytilus edulis (Mefp 1) (FIG. 1C). It is comprised of over 70 repeats of a decapeptide, which encompass most of the protein sequence. Here, the repeat decapeptide itself was used as a building block to evaluate sequence dependence and property enhancement when displayed on a dense polymer scaffold.

Example 1.1—Synthesis Methods: Ring-opening metathesis polymerization (ROMP) via a functional group tolerant modified second generation Grubbs' ruthenium initiator was used to generate a collection of polymers from peptide-modified, norbornene based monomers (FIGS. 2A-2F). Monomers, including amino acid sequence and polymerizable group, were prepared via SPPS, cleaved, purified and then mixed with initiator to form PLPs (FIG. 2A). A norbornenyl-peptide monomer was synthesized based on the repeating sequence of Mefp 1, described in Dalsin et al., Mussel Adhesive Protein Mimetic Polymers for the Preparatioono of Nonfouling Surfaces, J. Am. Chem. Soc. 2003, 125 (14), 4253-4258, which is hereby incorporated by reference. The repeating sequence being: Ala-Lys-Pro-Ser-Tyr-Hyp-Hyp-Thr-DOPA-Lys (Hyp stands for the oxidized proline form, 4-hydroxyproline). Three distinct degrees of polymerizations were targeted by adjusting the ratio of monomer to initiator to prepare PLPs of varying molecular weights (PLP-S, -M, and -L for small, medium, and large, respectively) (FIG. 2B). Additional control monomers were prepared by altering the tandem sequence: (1) PLP-Y-similar to the original sequence but with the oxidized amino acids (Hyp and DOPA) replaced by their native counterparts (Pro and Tyr respectively) (FIG. 2C); (2) PLP-GS—the original sequence was replaced with non-functional repeats of Gly and Ser to conserve solubility, but the DOPA residue kept at its prime position (FIG. 2D). While the first control was designed to examine the influence of the non-canonical amino acids, in particular DOPA, on the adhesion of the resulting polymer, the latter was meant to explore the contribution of the rest of the sequence. The polymerization was monitored by NMR and terminated as complete consumption of the monomer was observed. The products were then subjected to size-exclusion chromatography, with multiangle light scattering (SEC-MALS) to determine their molecular weights (FIG. 2E).

Example 1.2—SDS-PAGE Methods and Results: The PLPs at three degrees of polymerization were subjected to electrophoresis and separated relative to their respective mass by SDS-PAGE. Notably, clear separation of the polydisperse PLPs into individual bands, potentially representing specific degrees of polymerization (DP), was evident in the lower mass ranges (FIG. 2F). Interestingly, individual bands of higher DP derived from PLP-S lined up with bands of lower DP of PLP-M as those should be essentially identical molecules (FIG. 2F).

Across the series, the PLPs appear slightly higher in molecular weight by SDS-PAGE analysis compared to the SEC data, although they do not necessarily run precisely with the molecular weight ladder which is entirely protein-based (Precision Plus Protein, Bio-Rad). Therefore, we assigned DP and other parameters used to describe the polymers from the more traditional SEC data (FIG. 2E).

Example 1.3—AFM Methods: To assess the ability of the polymers to adhere to a solid substrate, atomic force microscopy (AFM) was employed. By repeatedly oscillating the AFM cantilever, the force-distance curves were measured and analyzed to give the respective adhesion value of each point on the surface. Briefly, silicon wafer chips were coated with the different PLPs to the same density and were scanned for adhesion force by AFM. Chips covered with the native protein Mefp 1, and the tandem repeat decapeptide were used for comparison. Overall 50 distinct points covering different areas of the coated surface were sampled for each specimen.

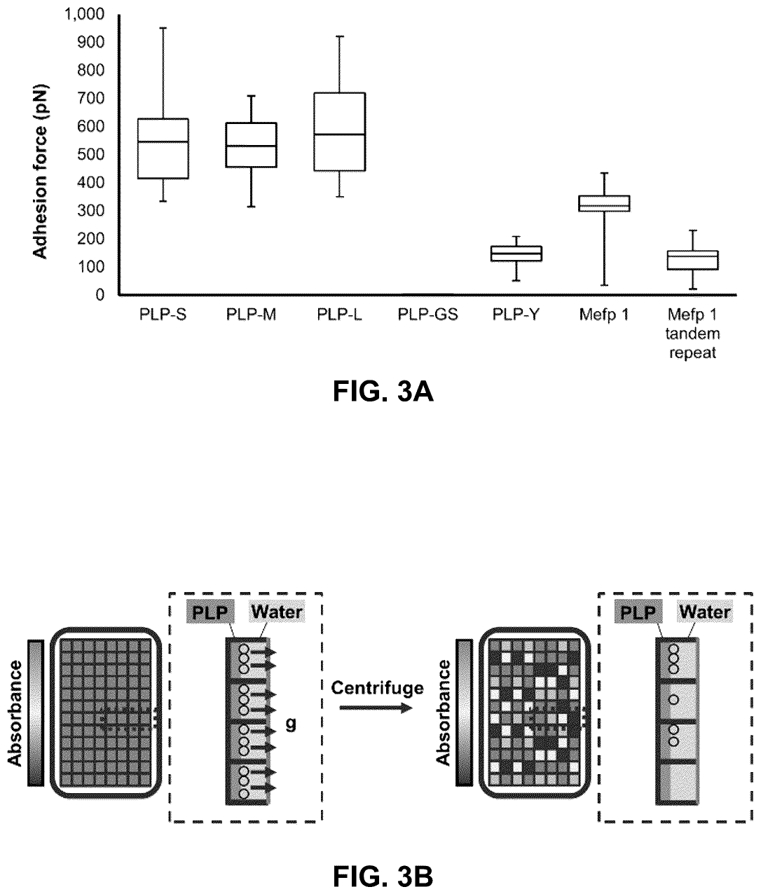

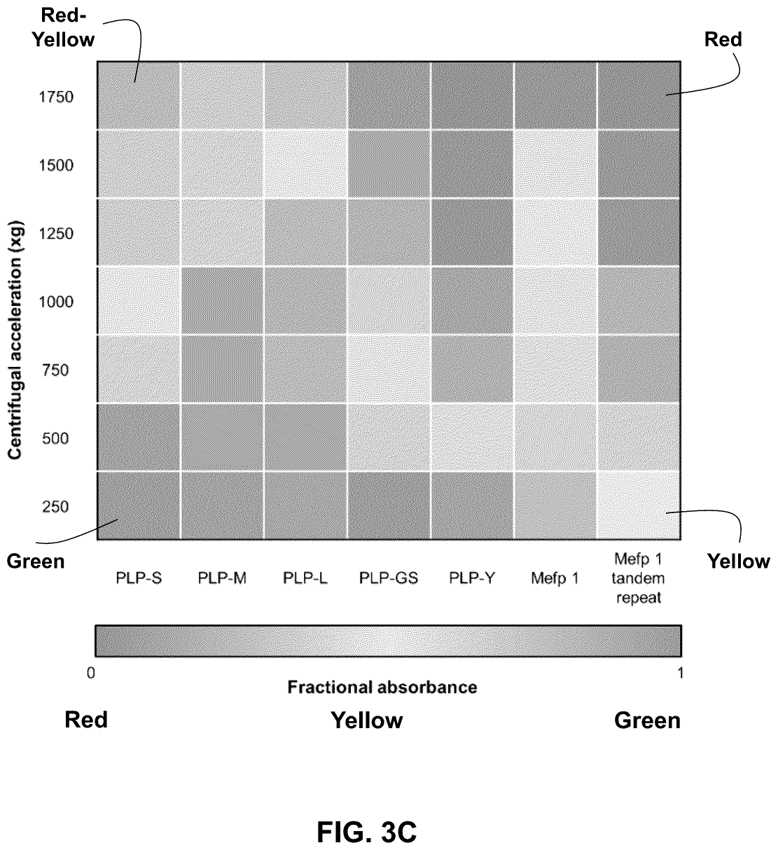

Example 1.3—AFM Results: These results revealed that the three degrees of polymerization tested in this study resulted in very similar values of adhesion force with a median above 500 pN (FIG. 3A). The results shown in FIG. 3A reveal no significant difference between the 3 tested degrees of polymerization. The non-oxidized control, PLP-Y, displayed values close to those of the short tandem repeat peptide. By contrast, the adhesion value for the DOPA-containing control, PLP-GS, was close to zero—attributable to the fact this PLP was susceptible to oxidization. It is known that the proteinaceous environment surrounding the DOPA residues in Mefps can prevent their spontaneous oxidation. It has also been shown that the synergy between DOPA and other amino acids, mainly Lys, is critical for marine adhesion. Finally, the native protein showed the highest adhesion force values among the examined controls, which reached approximately half those measured for the synthetic PLPs (FIG. 3A).

Example 1.4—Centrifugal Adhesion Methods: In parallel, the adhesion of the different compounds was compared using a centrifugal adhesion test (FIG. 3B). The method characterized the detachment status of each well at different centrifugal speeds as a proxy for the strength of the adhesive materials. Stronger adhesive formulation retain more particles compared to the less adhesive counterpart after centrifugation at a certain speed. Well bottoms were coated with the different PLPs at the same density as each control sample. Microparticles (radius of 5 μm) submerged in water were pipetted into each well and centrifuged onto the adhesive films to ensure good contact. The multi-well plate was then subjected to different centrifugal speeds with the particles facing up and outward. Particle detachment was evaluated by absorbance measurements between centrifugations. The average fractional absorbance was calculated from six replicates on the same multi-well plate (FIG. 3C).

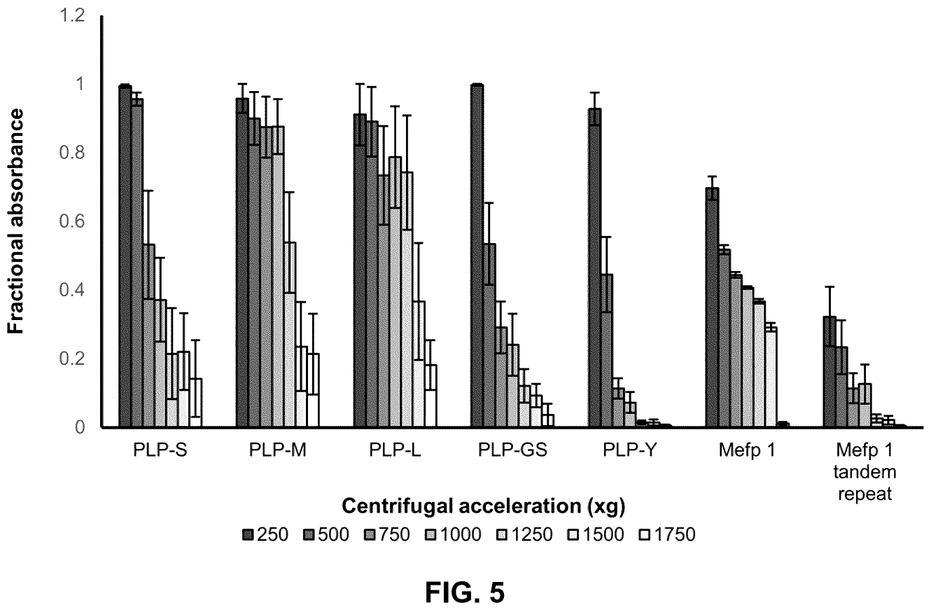

Example 1.4—Centrifugal Adhesion Results: The results obtained with this test (FIG. 3C and FIG. 5) were in agreement with those observed by AFM. Testing the adhesion behavior of the materials at various speeds allowed for better discernment of the adhesive properties of the three different molecular weight PLPs containing the active repeat sequence, which was not possible through the adhesive force measurements. Specifically, as the centrifugation speed was increased, more particles remained attached in the wells coated with the higher degree of polymerization PLPs (PLP-S<PLP-M<PLP-L) (FIG. 3C). All three adhesive PLPs again showed better adhesion than control PLPs, native protein, and the tandem repeat peptide alone.

The superior performance observed for the PLPs in both tests is believed to be attributed to the high density display of the functional unit in the polymer brush setting. This is compared to the protein where many of the repeats are either scrambled or partial, and possibly not exposed to the surface due to protein folding.

### Example 2

This example used the PLPs prepared in Example 1 to further evaluate its adhesion properties compared to the natural Mefp 1 TRPs.

The typical current commercial application of mussel adhesive proteins is as coatings of substrates used to immobilize cells and tissues. It can simplify the manipulation of biological samples in numerous common in vitro techniques including establishment of primary cultures, in situ hybridization, immunoassays, microinjection and immunohistochemistry. Since mussel adhesive proteins can form strong adhesion with various substrates such as glass and plastic, work in wet environments, and are biocompatible and non-toxic they are ideal for cell immobilization and in theory can even serve as a biological glue for various surgical and dental applications. However, as previously mentioned, these proteins are difficult to produce and are hence limited in utility.

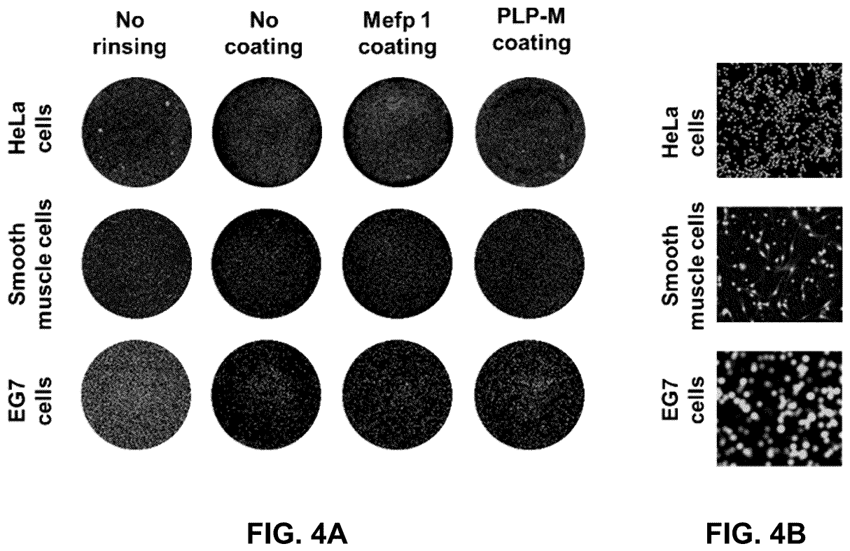

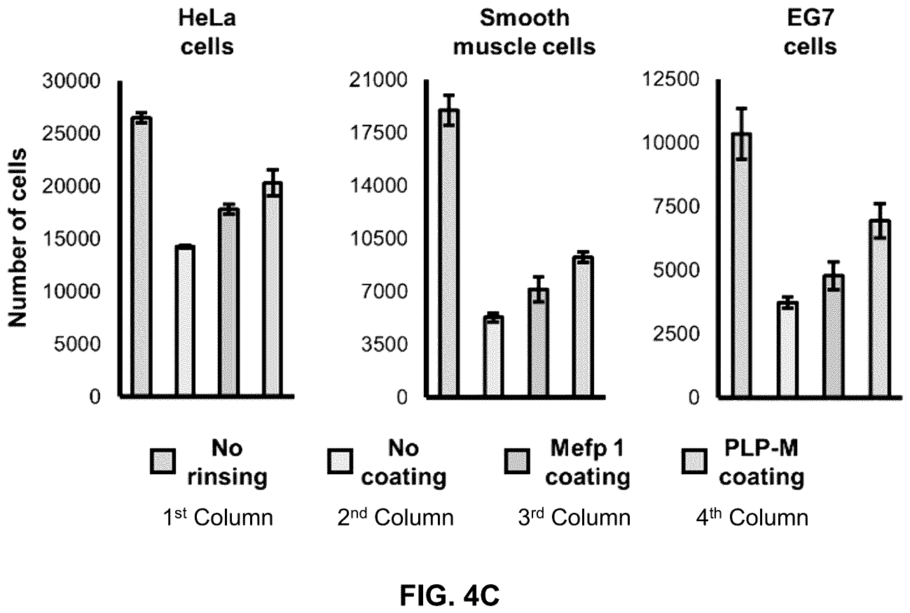

Example 2.1—Immobilization Methods: The ability of the adhesive PLPs to immobilize live cells was examined using three different cell lines chosen based on their adherence properties (FIGS. 4A-4C): (1) HeLa cells, a robust cell line that has an intrinsic ability to adhere to surfaces; (2) aortic smooth muscle cells (SMCs) which are reliant on external biomaterials such as collagen for binding substrates; (3) EG7-OVA, lymphoma cell line which is cultured in suspension (FIGS. 4A-4C).

Example 2.1—Immobilization Results: The SMCs in particular show the medical potency of the material, as these cells line the blood vessels. Hence, the polymer could potentially be used to patch arteries without the need of stiches. The EG7 immortalized line of T lymphocyte cells are often used as a model system for studying major histocompatibility complex (MHC) class I restricted responses of cytotoxic T lymphocytes. For that purpose these cells have to be immobilized, which is currently done using harmful reagents such as aldehydes.

Example 2.2—Cell Adhesion Assay Methods: Cell adhesion assays were performed in non-coated glass-bottom plates following treatment of the wells with either the adhesive PLP-M or the native protein. Wells that were left untreated were used as a negative control. Following seeding of cells and a 12 hour-incubation time, the wells were rinsed to wash unattached cells, and stained with Hoechst to detect live cells (FIGS. 4A-4B). The plates were scanned and the number of cells in each well was analyzed by automated image processing.

Example 2.2—Cell Adhesion Assay Results: The same trend was observed for each of the three cell lines. Specifically, wells coated with PLP-M showed adhesion of more cells than those coated with the protein, which in turn as expected, exhibited higher cell adhesion than the untreated wells (FIG. 4C). The number of cells in untreated wells that were not rinsed was displayed to provide a baseline of the total number of live cells present, either attached or not.

### Example 3

In this example, preparation and characterization of secondary structure-inducing peptides as PLPs is provided. Specifically, designed ankyrin repeat proteins (DARPin) is investigated.

Though synthetic TRPs have shown success as chemical biology tools and therapeutics, they suffer from notable drawbacks. Mainly, to be used for applications in health and biology, they must be expressed in cells via costly recombinant methods, thus limiting their scalability and range of application. Although the utility of the DARPin platform is high, continued research effort has focused on novel strategies for repeat proteins and protein engineering. As a result, we envision that a strategy to develop proteomimetics of ankyrin repeat proteins (FIGS. 13A-13C), which will take advantage of synthetic chemistry to rapidly synthesize and establish properties. It is expected that this will lead to further advances in proteomimetics of repeat proteins, both fundamentally and as tools for targeting PPIs in assays and in the clinic.

Example 3.1—Proof of Concept Methods: Proof-of-concept studies were performed to determine fundamental properties of structured peptides incorporated into brush polymers. Specifically, there are few previous studies that have characterized the behavior of brush polymers with alpha helices and none with other secondary structures (i.e. β-hairpins). However, to proceed with accurate mimicry of the structured units that are found in ankyrin repeat proteins, it is relevant to understand the behavior of simpler constructs. Along those lines, a small library of polymers was synthesized, utilizing peptide macromonomers inspired by previous reports of a crystal structure of an alpha helical peptide, as well as a highly stable, water-soluble β-hairpin peptide. These brush polymers were compared to those constructed using an unstructured, random coil peptide. Varying lengths of polymers from 15 to 45 repeats (19 kDa to 84 kDa depending on the peptide), were synthesized and characterized.

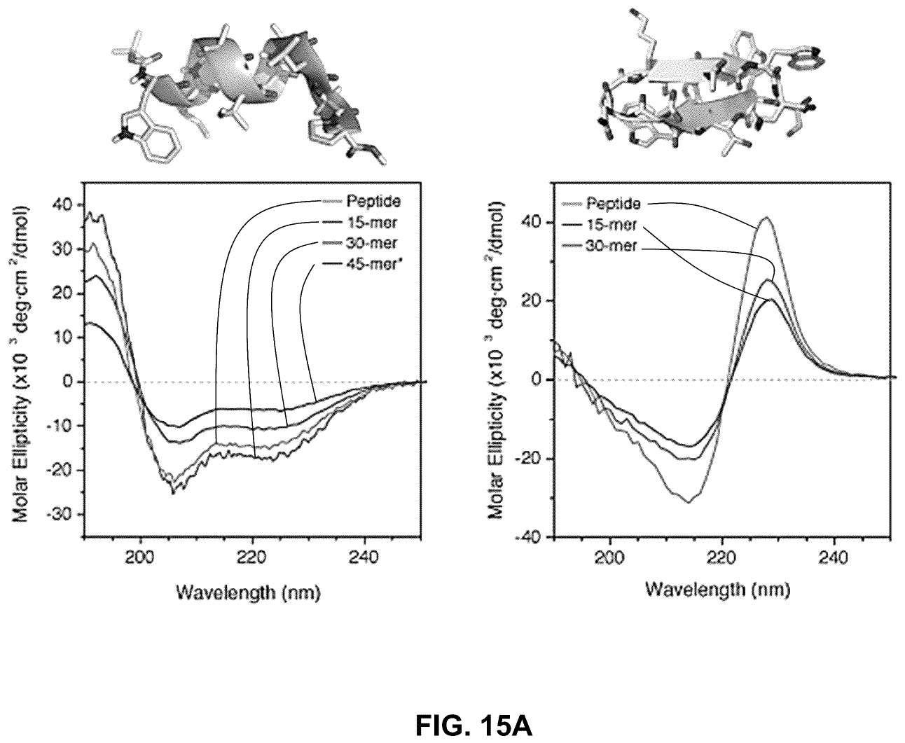

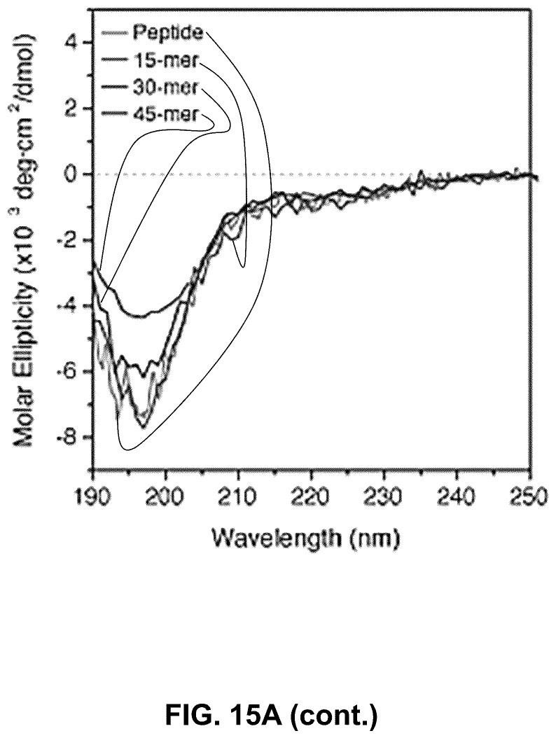

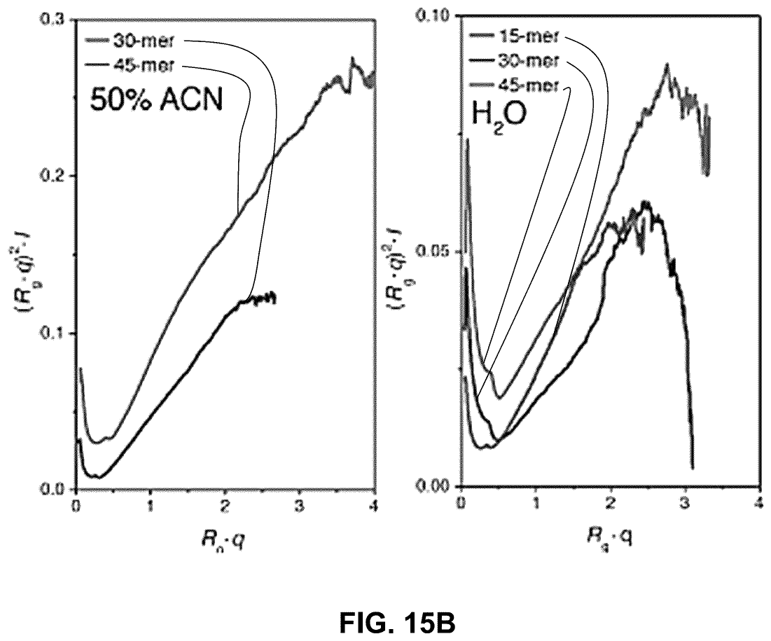

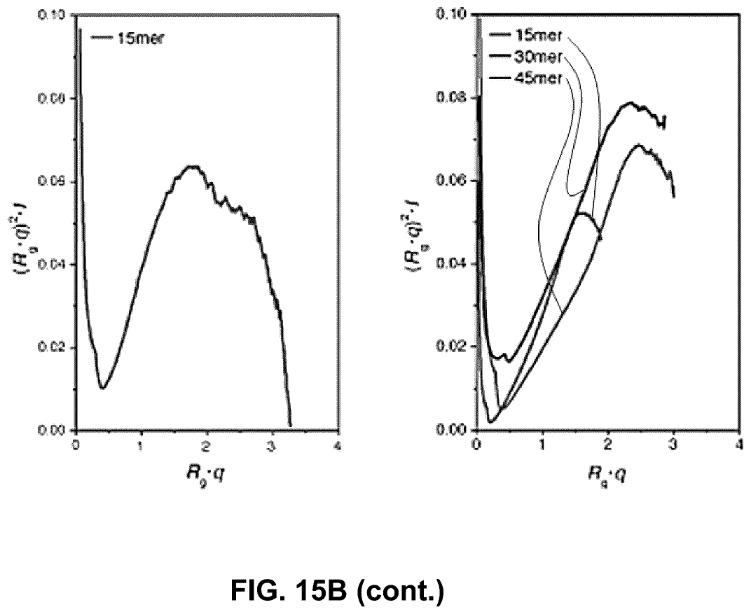

Example 3.1—Results: Circular dichroism (CD) spectroscopy confirmed that the peptides on the resulting polymers have similar structures to those of the monomer, confirmed by similar features in their CD spectra (FIG. 15A). However, the longer the polymers, the greater the reduction in intensity of their CD spectra in aqueous solvents, which can be attributed to an artefact from the clustering of peptides onto polymers (akin to that observed in membrane proteins with detergents). SAXS studies confirmed that longer polymers became increasingly more rod-like in shape for most classes of samples, with the smoothed Kratky plots showing that the samples are extended towards the upper right of the plots, indicative of less globularity (FIG. 15B).

Example 3.2—Solvation of Polymer Backbone: The competition between solvation and hydrogen bonding of the peptide side chains was studied as well as solvation of the polymer backbone. Previous studies on PLPs demonstrated that the solvation of the polymer backbone plays an important role in the structure of the resulting PLPs, with hydrophobic polymer backbones resulting in more globular polymer conformations in water.

Example 3.2—Results: Helical PLPs whose hydrogen-bonding is disrupted by water were more globular with more water present, as evidenced by the pronounced downturn in the SAXS data in 100% H₂O samples (which clearly turn-over in slope compared to 50% acetonitrile samples, FIG. 15B). These studies demonstrate key concepts needed for the implementation of the proposed research: specifically, that PLPs can support secondary structure elements on the polymer backbone without significant perturbation of the hydrogen-bonding patterns crucial for folding. This preliminary work suggests it is possible to polymerize even more complex arrangements of secondary structure. Additionally, the influence of peptide properties on the PLP's solution-phase shape has been demonstrated, indicating that PLPs will be able to form elongated shapes akin to ankyrin repeat proteins.

### Example 4

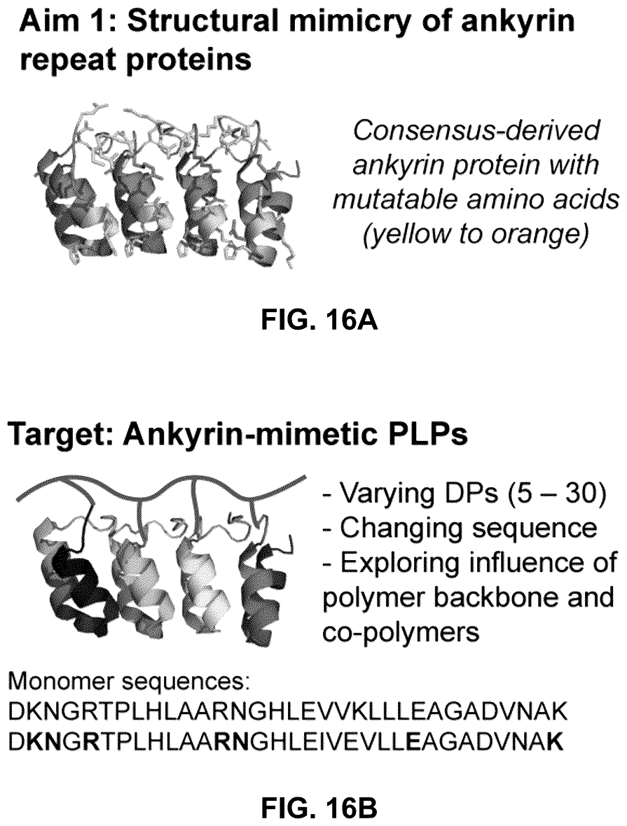

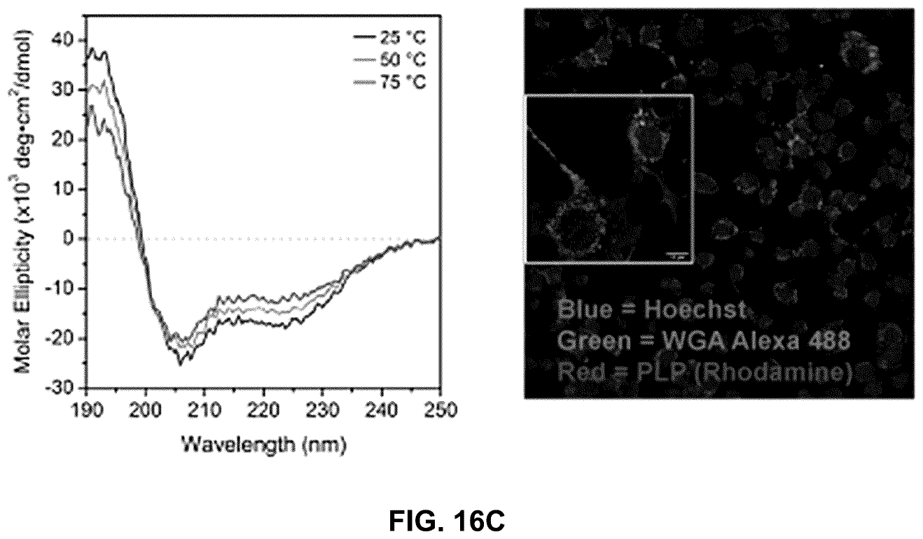

In this example, structural mimics of ankyrin repeat proteins by polymerizing consensus-derived ankyrin repeats on a brush polymer scaffold is explored (FIG. 16A-16C).

The goal was to build proteomimetic polymers out of ankyrin repeat units. It was hypothesized that such systems would display analogous, biomimetic structural properties to natively-constructed proteins, which was supported by previous investigations of ankyrin proteins, as well as preliminary work described in Example 3. Specifically, reports identified averaged sequences of the ankyrin domain which, when expressed as three or more repeats, fold into the characteristic helix-loop-helix motif. These consensus-derived sequences were the first targets: DKNGRTPLHLAARNGHLEVVKLLLEAGADVNAK and DKNGRTPLHLAARNGHLEIVEVLLEAGADVNAK, where the bolded positions are frequently mutated.

Example 4.1—Synthesis and Chemical Characterization: Ankyrin-based peptide sequences were synthesized and purified by SPPS. The norbornene unit was coupled to the peptide on resin via established amide-bond forming chemistry, and the resulting peptide was deprotected and purified using established methods. The purified peptide monomer was then polymerized by ROMP to form brush polymers where the side chains of the polymer were the ankyrin repeat peptide. The polymerization was monitored by NMR and terminated as consumption of the monomer was observed.

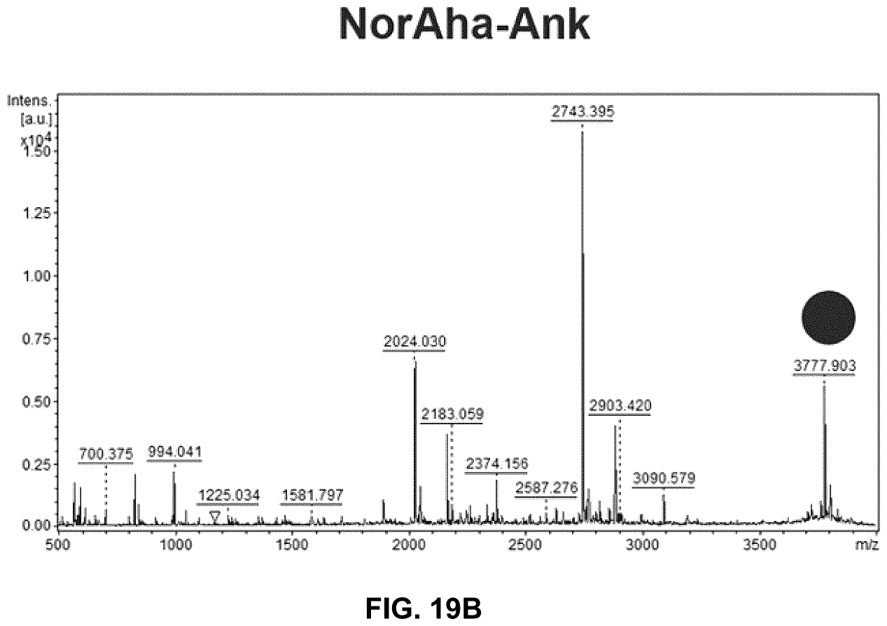

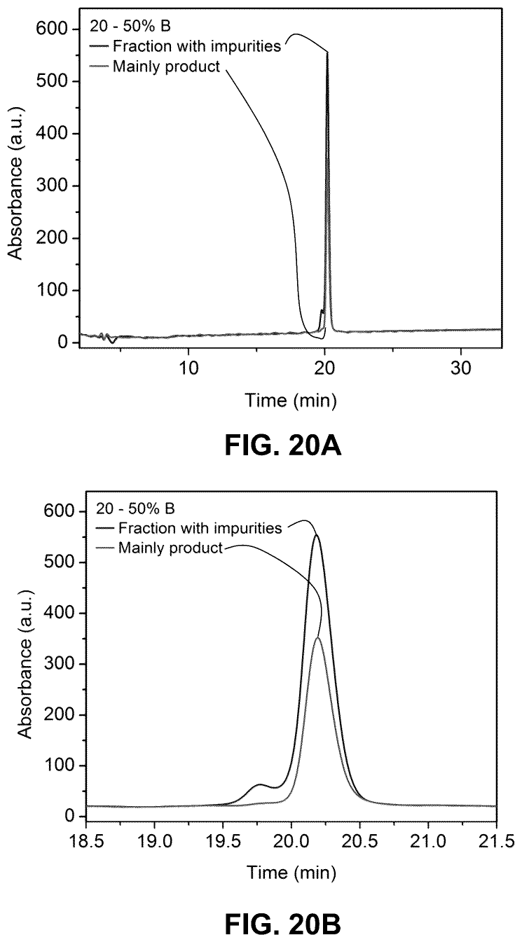

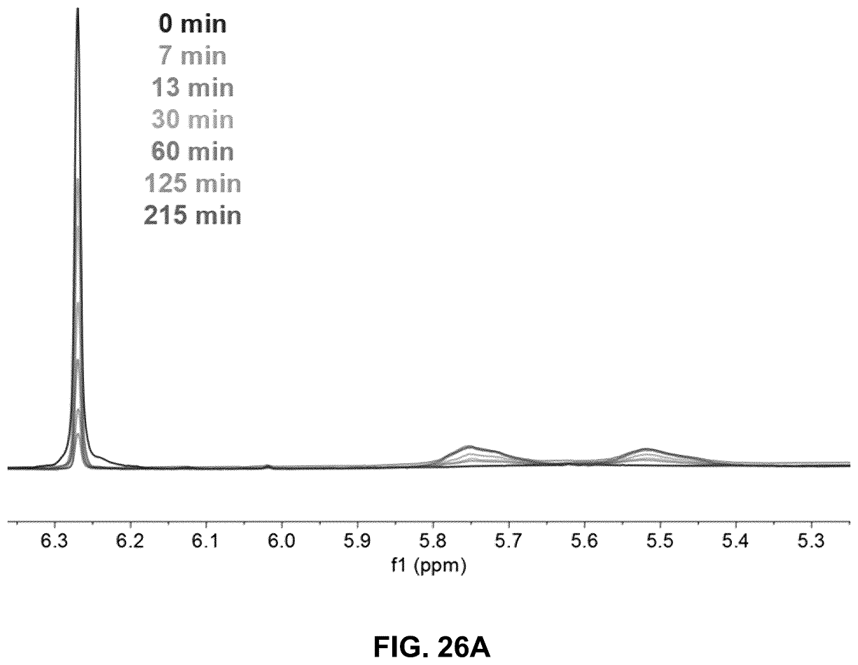

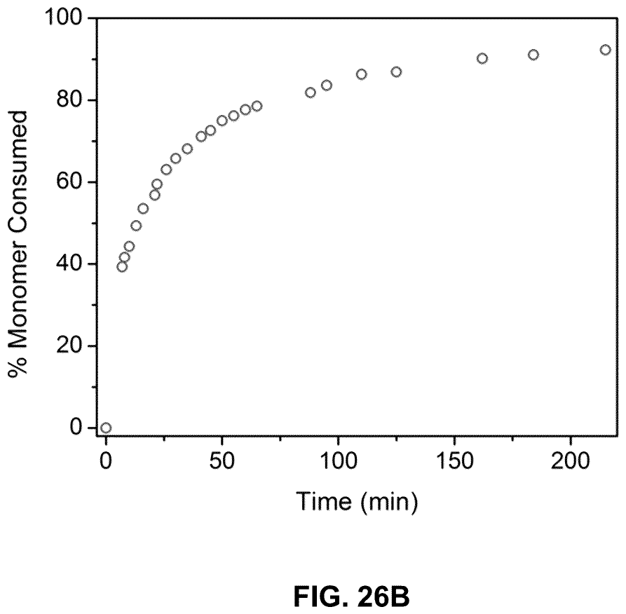

Example 4.1—Results: Synthesis of the functionalized peptide having the sequence DKNGRTPLHLAARNGHLEVVKLLLEAGADVNAKDK was confirmed by mass spectrometry as shown in FIG. 19A and FIG. 19B. The resulting ankyrin monomer had no appreciable impurities as shown in FIGS. 20A-20B. FIGS. 26A-26B depict the polymerization kinetics for ankyrin-PLPs with the increasing ankyrin monomer consumption suggesting successful polymerization.

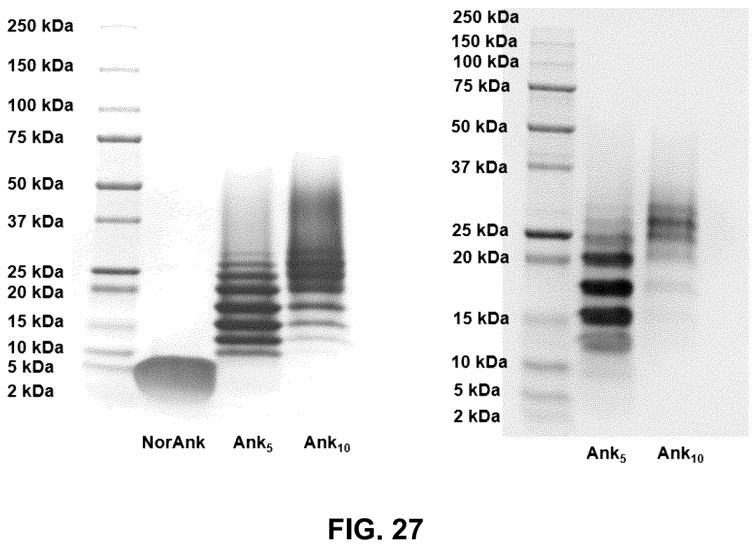

Example 4.2—SDS-PAGE Methods and Results: The PLPs at three degrees of polymerization (NorAnk, Ank₅, Ank₁₀) were subjected to electrophoresis and separated relative to their respective mass by SDS-PAGE. Notably, clear separation of the polydisperse PLPs into individual bands, potentially representing specific degrees of polymerization (DP), was evident in the lower mass ranges (FIG. 27).

Example 4.3—Structural Characterization of Purified Ankyrin Monomer: The ankyrin monomer without NorAha was studied using CD spectroscopy (50 μm in 10 mM NaPO₄ buffer, pH of 7, 20° C.) to determine the conformation and stability of the pendant ankyrin repeats. Previous studies have shown that single ankyrin repeats are unfolded, but once they are covalently linked together the close contacts between ankyrin domains promote folding into helices. Melting temperature of the ankyrin monomer was compared to melting temperatures of natural ankyrin peptides. The rate of temperature change was also tested to determine impact on stability of the ankyrin structure.

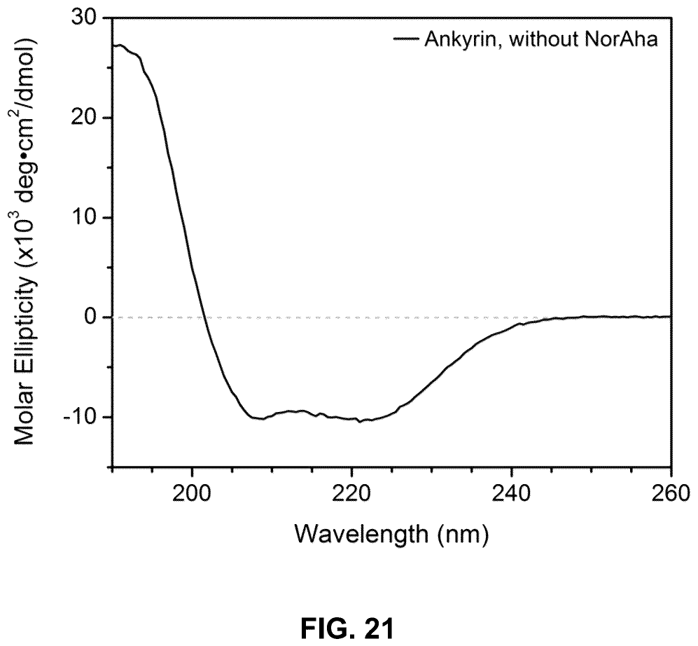

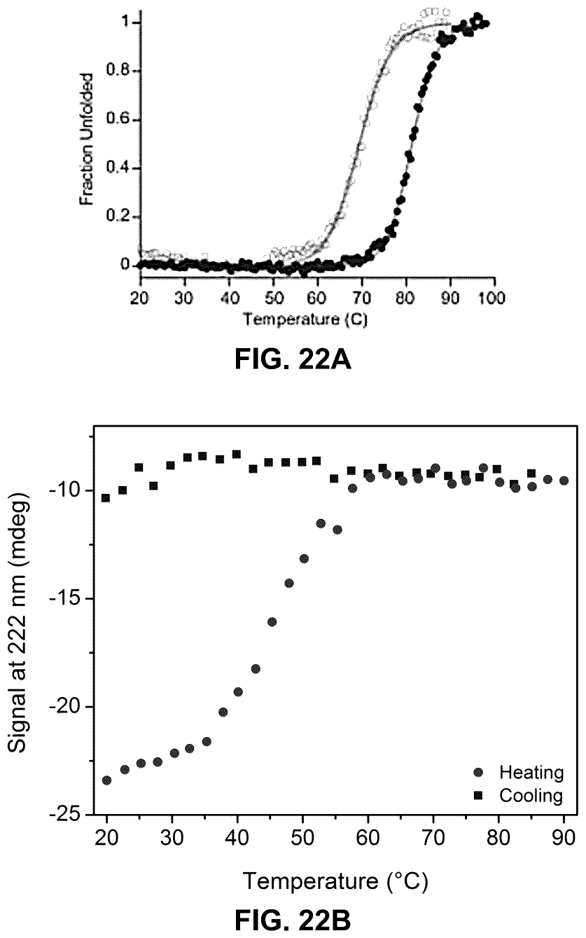

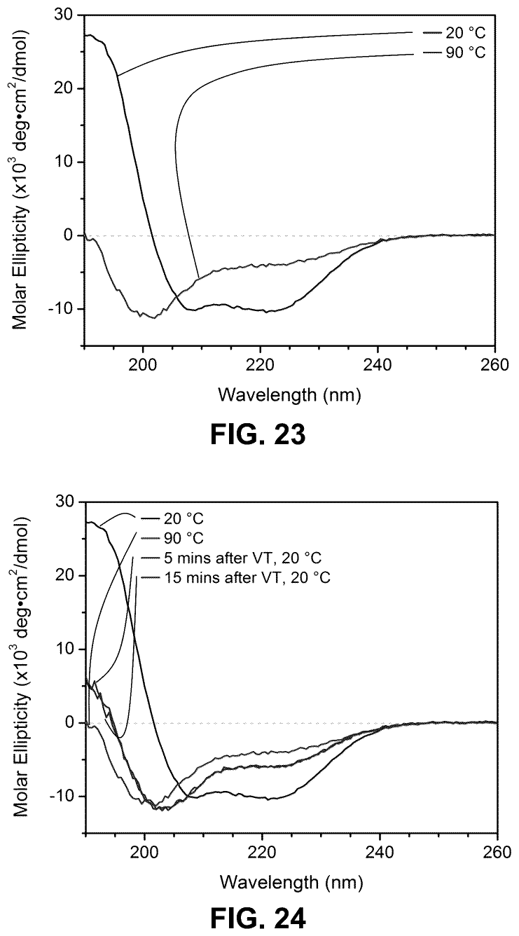

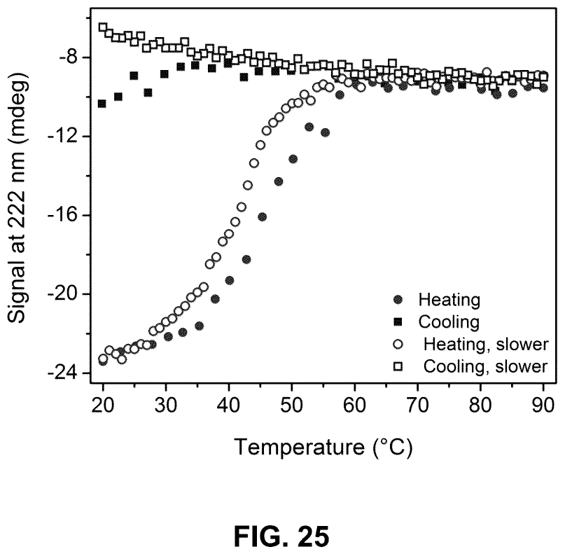

Example 4.3—Results: CD spectra results (FIG. 21) show a clear helical signal for the natural ankyrin peptide without NorAha (i.e., not bound to the norbornene-amino hexanoic acid (NorAha) linker), suggesting that it possesses at least some structural stability as a monomer. FIG. 22A shows exemplary melting temperature of natural 3-repeat and 4-repeat ankyrin peptides adapted from Mosavi et al., Consensus-Derived Structural Determinants of the Ankyrin Repeat Motif, Proc. Nat'l Acad. Sci 2002, 99 (25), 16029-16034, which is hereby incorporated by reference. The melting temperature of the purified monomer prepared in Example 4.1 is consistent with said examples as shown in FIG. 22B. However, FIG. 22B also suggests that melting may result in irreversible denaturation of the monomer. The CD spectra results in FIGS. 23 & 24 similarly suggest the loss of helical content at higher temperatures, displaying characteristic peaks more consistent with random coils as opposed to alpha-helices. Reducing the rate of temperature change had little impact on the irreversible loss of helical content as shown in the CD spectrometry results in FIG. 25.

Example 4.4—Improving Stability via PLP Incorporation and Acidic Buffer: Stability of the ankyrin monomer after incorporation in the PLP platform was tested. Synthesis and polymerization into the PLP platform were conducted via the methods described in Example 4.1. The stabilities of NorAnk (i.e., not polymerized) and NorAnkio (i.e., 10 monomer units) were compared via CD spectrometry. The stability and solubility of the peptides in different solvents, acetate buffer and TFA, was also compared, with the CD spectrometry parameters being 50 μm in 10 mM acetate buffer, pH of 5, 20° C. (after VT) and 50 μm in 13 mM TFA buffer, pH of 2, 20° C. (after VT).

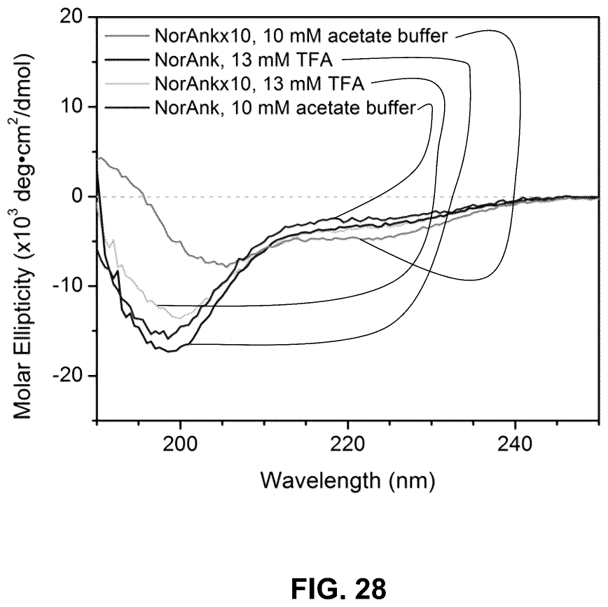

Example 4.4—Results: As shown in FIG. 28, the NorAnkio in acetate buffer reflects the characteristic peaks for alpha-helical structure, which is not reflected by the other tested samples. This suggests that a combination of acetate buffer and polymerization lead to structural recovery after heating. It appears that both the solvent and the PLP platform support improved stability. With respect to solvent, both the NorAnk and NorAnkio samples in TFA buffer show significantly less helical structure recovery as compared to the acetate buffer counterparts, which is demonstrated by the lower positive intensities at ˜190 nm. Regarding polymerization supported by the PLP platform, NorAnk10 shows a higher helical signal (i.e., a higher positive intensity at ˜ 190 nm) than NorAnk in both solvents. Therefore, the solvent and the PLP platform independently support structural stability of ankyrin. While not shown, it was also observed that solubility of the NorAnkio was improved at lower pH levels, namely pH of less than 5.

Example 4.5—Improving Stability via Elongation and Catalysts: It is hypothesized that the stability of the natural ankyrin peptide helical structure may also be improved by polymerization into longer constructs (>15 monomer units, or >55 kDa) in order to allow for greater stabilizing contacts between ankyrin repeats. Longer polymers will be generated and tested using the methods described herein. Additionally, catalysts providing more control of the peptide's stereochemistry will be explored, such as Z-selective catalysts as described in Khan et al., Readily Accessible and Easily Modifiable Ru-Based Catalysts for Efficient and Z-Selective Ring-Opening Metathesis Polymerization and Ring-Opening/Cross-Metathesis, J. Am. Chem. Soc., 2013, 135 (28), 10258-10261, which is hereby incorporated by reference.

Example 4.5—Prophetic Results: SAXS and dynamic light scattering (DLS) will confirm the approximate solution-phase size and hydrodynamic radius of our samples as compared to native ankyrin repeat proteins. Analysis of SAXS data is expected to confirm that the PLPs are elongated rods consisting of aligned domains of ankyrin repeats. It is anticipated that results similar to those seen in FIG. 21 will be observed in the proteomimetic platform, with longer polymers (>3 monomers) leading to more structured systems. Longer polymers (>15 monomer units, or >55 kDa) may also prove observable using electron microscopy, particularly cryoTEM. The stability of the folding of such polymers will be confirmed using CD spectroscopy studies, where the loss of helical content will be observed versus sample temperature and under influence of denaturants (urea or guanidinium hydrochloride). This data will be used to quantify the thermodynamics of the folding of the PLP, which can be compared to extensive studies in ankyrin repeat proteins. Differential scanning calorimetry (DSC) will be used to provide complementary information into secondary structure composition and stability as a function of temperature and solvent environment.

Variable-temperature SAXS and DLS studies will be conducted under similar conditions to confirm that unfolding of the PLPs is accompanied by changes in the solution-phase shape. The expected outcome of these studies will be determination of synthetic compositions necessary to achieve stably folded PLPs with structural properties akin to ankyrin repeat proteins.

Example 4.6—Optimization of Ankyrin-PLP System: A key aspect of developing and understanding these systems will be exploring the influence of the polymer backbone on the construct. Peptides will be synthesized where the norbornene will be attached at different points along the ankyrin repeat (i.e. variable positions along the β-hairpin of the repeat or at the loop position). These modifications will mean that once polymerized, the polymer backbone will be a spatially distinct position relative to the ankyrin domain. Additionally, the influence of mutating specific residues known to be necessary for maintaining the ankyrin repeat structure on the resulting PLP's stability will be pursued. Of interest in particular is the disruption of hydrophobic regions (i.e. DKNGRTPLHLAARNGHLEVVKLLLEAGADVNAK). For all peptide-based monomers, lengths of polymers from 5 to 30 repeats for chemical, structural, and biological characterization will be synthesized. Polymerization will be performed with non-stereoselective ROMP catalysts as well as stereoselective catalysts to confirm the influence of stereochemistry on the properties of the resulting PLPs. The co-polymerization of ankyrin repeats with flexible peptides (i.e. GSGSGS) will also be explored to evaluate how spatial dilution of the ankyrin repeat affects the resulting polymer properties.

Example 4.7—In Vitro Characterization: Once successful ankyrin PLPs have been generated, critical properties necessary to employ these as chemical biology tools will be examined. As many ankyrin PPIs are intracellular, one of the most important properties of these PLPs will be their cellular uptake ability. Many other PLPs, including ones of similar size to those being proposed (˜30 kDa), have been readily delivered into cells using the addition of arginines to the peptide sequence, or by creating copolymers with cell-penetrating peptide monomers (e.g. GRKKRRQRRRPQ, also known as TAT). However, it is unclear if similar strategies would work for these proposed polymers. In particular, the proposed PLPs will be less disordered than previously explored examples, so they may not be able to interact with the cell membrane to facilitate endocytosis and later endosomal escape. As a result, it will be important to examine cellular uptake of fluorescently labelled PLPs, which will be made by incorporation of a fluorescein- or rhodamine-based monomer or chain-terminating agent, depending on the experiment, in representative cell lines (i.e. commercially available HeLa cells). Confocal microscopy studies will confirm cellular uptake by observing whether the fluorescent tag of the PLP is visible in treated cells, as well as confirm whether such ankyrin repeat PLPs are capable of endosomal escape. These experiments will be conducted in the Biological Imaging Facility at Northwestern. Cellular uptake of dye-labelled PLPs will be assessed quantitatively using flow cytometry using an in-house instrument and standard operating protocol. Initial PLPs, as well as those incorporating multiple arginines and those made as a block co-polymer with TAT and/or with GSGSGS, will be examined to determine the utility of ankyrin repeat-based PLPs in accessing cellular targets. The expected outcome of these studies will be the determination of principles that influence the cell penetration of ankyrin-based PLPs.

Example 4.8—Additional Expected Outcomes and Alternative Methods: It is anticipated that this combination of structural studies and cell penetration assays will yield highly stable, folded ankyrin mimics that can penetrate cells for use in intracellular assays. In addition to the stability issues noted in Example 4.3, it is also conjectured that solubility of the monomer may prove an issue in purifying and polymerizing our targets. To address these stability and solubility challenges, changes in the ankyrin repeat sequence in positions that can accommodate mutations without endangering its helical structure will be pursued. Particular amino acids (indicated by bolded positions in the sequences above) will be swapped out for more hydrophilic residues, while still maintaining the amino acids necessary for the helical platform. If such changes are not adequate for stability and/or solubilization of the peptide, the coupling of oligo (ethylene glycol) (OEG) units to the repeat domain, using an OEG-incorporating norbornene linker will be employed. It is expected that these two strategies will successfully stabilize and solubilize peptides.

### Example 5

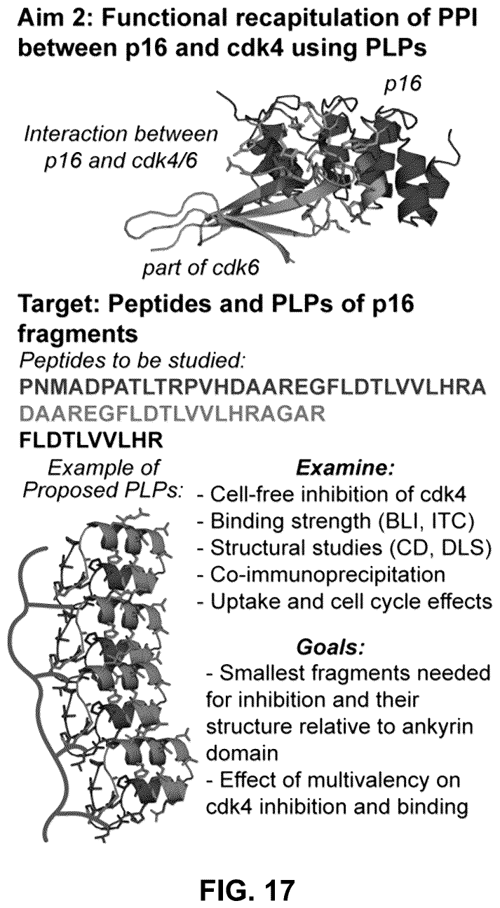

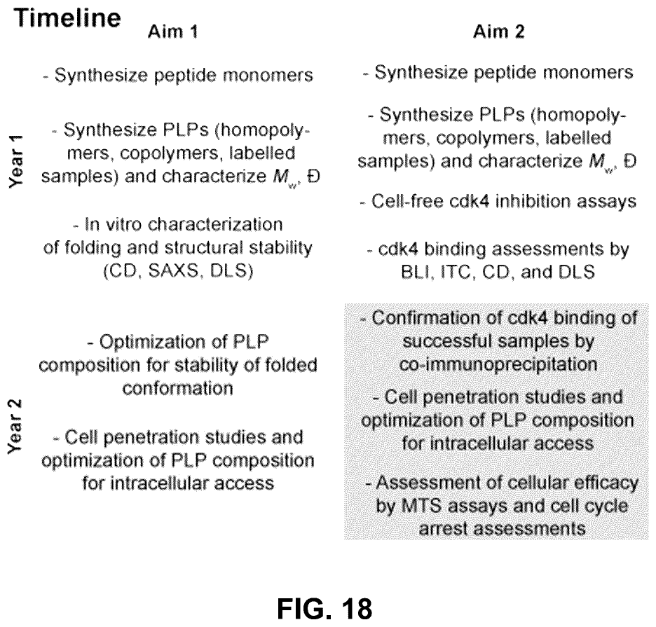

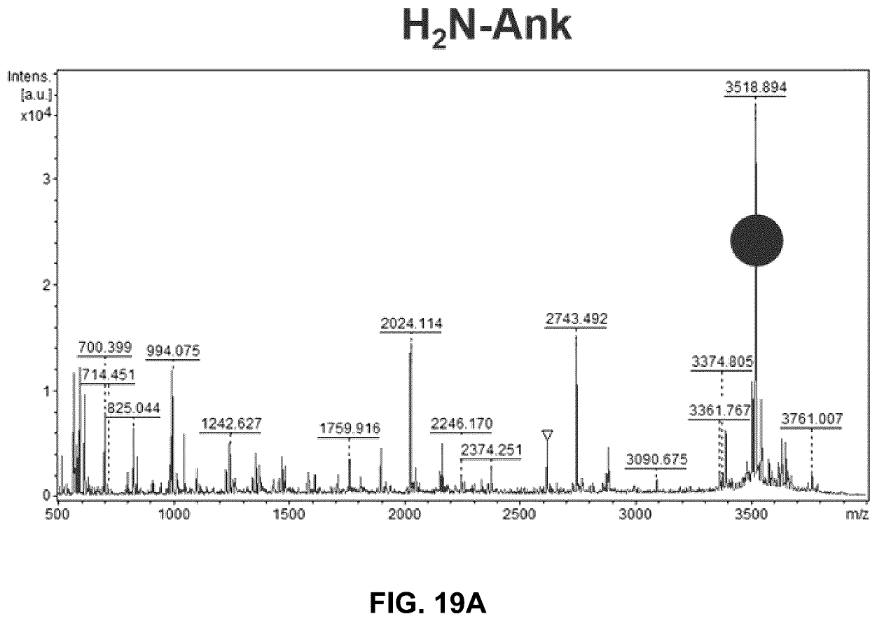

In this prophetic example, recapitulation of the functional behavior of the ankyrin repeat protein, p16 with cdk4 by multivalent presentation of p16 fragments on a proteomimetic polymer scaffold is explored (FIG. 17).

It is hypothesized that high-affinity PPIs promoted by ankyrin repeat proteins can be recapitulated by polymerizing ankyrin fragments on the PLP proteomimetic platform. To examine this, the test case of the tumor suppressor protein p16 will be probed, which consists of four ankyrin repeats and is involved in cell-cycle progression by inhibiting cyclin-dependent kinase 4 (cdk4). In certain cancers, such as familial melanoma and malignant mesothelioma, mutations in p16 render it unstable or incapable of cdk4 inhibition, contributing to tumor progression. Accordingly, cdk4 inhibitors have been found to be important tools in battling cancer, and several selective small-molecule cdk4 (which also act on the related cdk6) inhibitors have passed clinical trials and are now in use as chemotherapeutics in the treatment of HR-positive breast cancer. A portion of the interaction with cdk4 is located on the second and third ankyrin repeats, and previous work showed that short segments of p16's third ankyrin domain (10-20 amino acids long) replicated this behavior. These studies did not explore the mechanism of inhibition of such peptides relative to the full protein, or how these segments of p16 behaved structurally, in comparison to the folded native protein. The implications of this activity on the importance of the ankyrin scaffold were also not explored further, leaving numerous questions regarding how and whether p16 can be accurately imitated by peptide fragments.

This test case allows for exploration of an important question: whether multivalency (by repeating presentation of parts of p16 on the PLP scaffold) can improve the inhibition of cdk4 by p16 fragments, which will provide important insight into the evolution of ankyrin repeat proteins like p16. The results from exploration of this aim will prove as a key test concept whether PLPs can accurately mimic ankyrin repeat proteins functionally, in complement to Example 4, which will verify structural mimicry of such proteins. It is expected that this research will provide insight into the evolution of TRPs, which must have proceeded through competent repeats that more closely resembled each other, before diverging in composition through evolution.

Example 5.1—Synthesis and Chemical Characterization: Portions of p16 by SPPS: DAAREGFLDTLVVLHRAGAR, FLDTLVVLHR, and the second and third ankyrin domains (ALPNAPNSYGRRPIQVMMMGSARVAELLLLHGAE and PNCADPATLTRPVHDAAREGFLDTLVVLHRA respectively) will be synthesized. These will be coupled to the norbornene monomer unit and polymerized by ROMP. Validation of peptide synthesis will be performed using HPLC and mass spectrometry, and polymers will be characterized by SEC-MALS, SDS-PAGE, and DLS to confirm their molecular weight distribution, polydispersity, and hydrodynamic radius. A representative sample of polymer lengths will be pursued, such as 5, 15, 30, and 60 monomer units, to assess how greater repetition of a peptide affects PLP properties. Additionally, random and controlled block co-polymers of the second and third ankyrin domains will be made and characterized, and compared to homopolymers of each domain, to assess the properties of closer mimics to p16.

Example 5.2—Cell-free Characterization as a PPI Inhibitor: With peptides and PLPs in hand, inhibition of cdk4 will be examined through a variety of in vitro methods. Initial inhibition will be assessed using commercial 96-well cell-free assays to quantify cdk4 activity in the absence and presence of samples. Kinase-dependent fluorescence will be assessed for a panel of samples using a fluorescence plate reader, and the data will be used to obtain IC₅₀ values of the samples. The wild-type p16 protein, as well as an FDA-approved cdk4/6 inhibitor, palbociclib, will be used as positive controls. Peptides and polymers of a “scrambled” peptide sequence of the p16 fragment, which should be incompetent at cdk4 inhibition, as well as a nonfunctional peptide sequence (i.e. GSGSGS), will be used as negative controls that should not show inhibition of cdk4 and therefore no reduction in fluorescence. Inhibition of cdk4 activity will be compared to binding of cdk4 to samples using in vitro protein-binding studies, including bio-layer interferometry (BLI) and isothermal titration calorimetery (ITC), to assess the strength of the interaction between p16-derived peptides and polymers and cdk4. Due to multivalency, it is expected that PLPs will show enhanced binding and prolonged off-rates, superior to peptide controls. It is anticipated that peptides and PLPs that bind more tightly to cdk4 should lead to improved inhibition of cdk4 activity. BLI-based binding and dissociation curves will be determined from a series of concentrations to calculate binding affinities between peptides and PLPs to sensor-immobilized cdk4. ITC will be pursued to determine the thermodynamic parameters of binding of peptides or PLPs to cdk4. A critical aspect of understanding the interaction between p16-mimicking PLPs and cdk4 will also be structural studies of peptides and PLPs and any structural changes upon binding to cdk4. CD spectroscopy will be employed to understand the folding on such samples, as well as changes to cdk4 upon addition of p16-mimic peptides and PLPs. Additionally, DLS of PLPs with cdk4 will be used to determine the change in hydrodynamic radius upon binding, consistent with the formation of a stable complex. Instruments in the Keck Biophysics Facility of Northwestern will be used for these experiments.

The interaction between cdk4 and PLP or peptide samples will be assessed using similar methodologies to previous reports, namely, co-immunoprecipitation using lysates of cdk4-expressing cells. Given the facile labeling of the PLPs via addition of a tagged monomer unit or terminating agent, PLPs will be tagged with biotin and pulled down using a commercial streptavidin kit. Samples will then be run on SDS-PAGE, and confirmation of PLPs binding to cdk4 will be conducted using Western blotting, conducted at the Recombinant Protein Production Core at Northwestern. Samples that show promising inhibition and binding of cdk4 will be characterized for activity and binding against other ligands to p16, as well as related cyclin-dependent kinases. Specifically, interactions with cdk6 and GRIM-19, which are interaction partners of p16 will be explored, as well as cdk1 and cdk2 via cell-free inhibition assays and co-immunoprecipitation to confirm selectivity for p16-binding proteins. It is expected that these studies to demonstrate the selectivity of multivalent presentation of targeted fragments on PLPs, allowing such constructs to inhibit critical PPIs with high specificity, but without off-target reactivity.

Example 5.3—In Vitro Characterization: Cellular uptake of p16-derived peptides and PLPs will be assessed in commercial HeLa cells. Dye-labelled PLPs will be synthesized and applied to cells before assessing uptake of samples using flow cytometry and confocal microscopy as described in Example 4. Additionally, MTS assays will be used to generate dose-response curves to determine at what point do peptides and PLPs affect cell proliferation. Once suitable concentrations for activity have been determined for key samples, cell viability at those concentrations will be assessed, as well as cell cycle progression, as that is another indicator of cdk4 inhibition. The positive and negative controls detailed above for cell-free cdk4 inhibition will be used.

Example 5.4—Expected Outcomes and Alternative Methods: It is expected that PLPs with better binding will exhibit superior inhibition of cdk4 activity in vitro and significantly reduce proliferation of HeLa cells. It is also expected that longer polymers will show greater improvements in inhibition and cdk4-binding in comparison to peptides and shorter polymers, indicative that multivalency is improving PLP performance. PLPs that do not show promising uptake in cells will be made using different cell-penetrating compositions, such as the addition of arginines or incorporation of the TAT sequence as described for Example 4. If PLP samples show uptake into HeLa cells but do not affect cdk4 inhibition or cell death, the use of alternative cell lines demonstrated to have a loss of p16 activity to confirm this behavior will be explored. Finally, native p16 has shown tendencies towards structural instability that results in aggregation. If similar behavior in p16-mimicking PLPs is discovered, stabilization by asparagine β-hydroxylation of monomers will be employed, longer polymers as those should have improved properties will be pursued, and sequences with mutations identified to provide p16 with greater stability to unfolding will be used.

It is anticipated that results from pursuit of Examples 4 and 5 in parallel will lead to the development of a new class of tandem repeat mimics that will replicate the structural properties of ankyrin repeat proteins, as well as their functional behavior in high-affinity interactions with proteins. It is expected that the long-term goal, which is the precise design of ankyrin repeat-mimetic PLPs for the delivery of therapeutic proteomimetics and as chemical biology tools will be achieved. With the combined knowledge from these lines of inquiry, rules will be established for the creation of highly stable and effective PLPs that target PPIs in diseases. Using similar strategies, PLPs to replicate repeat proteins like those incorporating tetratricopeptide domains, beta-propellers, and leucine-rich repeats can be used. This knowledge will inform the design of proteomimetic materials for therapeutic applications, in addition to inspiring the application of protein design in polymer chemistry.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A polymer characterized by a formula (FX1): 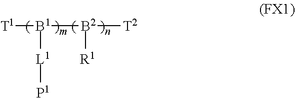
wherein:
each P¹ independently comprises a peptide;
at least one P¹ comprises: (i) a sequence having at least 75% or greater sequence identity of a tandem repeat peptide sequence found in a natural protein or natural peptide and/or (ii) at least one catechol residue;
T¹ and T² are each independently polymer backbone terminating groups that can be the same or different;
B¹ and B² are each independently polymer backbone subunits;
each L¹ is independently a linking group;
each R¹ is independently a substituent;
m is an integer selected from the range of 2 to 1000;
n is an integer selected from the range of 0 to 1000;
each connecting line in formula (FX1) represents a covalent linkage comprising at least one of a single bond, a double bond, one or more atoms, or any combination thereof, optionally, for example, each connecting line represents a single bond or double bond;
each instance of B¹, B², L¹, R¹, and P¹ is the same as or different from any other instance of B¹, B², L¹, R¹, and P¹, respectively; and
when n is an integer from 1 to 1000 and/or at least one instance of P¹ is different from another instance of P¹, the polymer is a block copolymer or a statistical copolymer.

2. The polymer of claim 1, wherein at least one P¹ comprises: (i) a sequence having at least 75% or greater sequence identity of a tandem repeat peptide sequence found in a natural protein or natural peptide or a modification thereof and (ii) at least one catechol residue.

3. (canceled)

4. The polymer of claim 1, wherein:
at least one P¹ comprises a sequence having 75% or greater sequence identity of a tandem repeat peptide sequence found in a natural protein or natural peptide; and
the tandem repeat peptide sequence is found in a mussel or a secretion thereof, a spider silk, a silk protein, an ankyrin, a collagen, a keratin, an elastin, a resilin, a squid ring teeth protein, a fibroin, or an abductin.

5. The polymer of claim 1, wherein;
at least one P¹ comprises a sequence having 75% or greater sequence identity of a tandem repeat peptide sequence found in a natural protein or natural peptide; and
the tandem repeat peptide sequence is found in mussel foot protein.

6. The polymer of claim 1, wherein;
at least one P¹ comprises a sequence having 75% or greater sequence identity of a tandem repeat peptide sequence found in a natural protein or natural peptide; and
the tandem repeat peptide sequence comprises or consists of the following ten amino acids:
(1) alanine, (2) lysine, (3) proline or 4-hydroxyproline, (4) serine, (5) tyrosine or 3,4-dihydroxyphenylalanine, (6) 4-hydroxyproline or proline, (7) 4-hydroxyproline or proline, (8) threonine, (9) 3,4-dihydroxyphenylalanine or tyrosine, and (10) lysine.

7. (canceled)

8. (canceled)

9. The polymer of claim 1, wherein at least one P¹ comprises a sequence having 60% or greater sequence identity of SEQ ID NO: 1 (AKXSXXXTXK), wherein each X at position 3, 6, and 7 is independently a proline or a 4-hydroxyproline, and each X at position 5 and 9 is independently a tyrosine or a 3,4-hydroxyphenylalanine.

10. (canceled)

11. The polymer of claim 1, wherein at least one P¹ comprises a sequence having 75% or greater sequence identity of SEQ ID NO: 16 (AKPSYPPTYK).

12. The polymer of claim 1, wherein
at least one P¹ comprises all D amino acids or all L amino acids.

13. The polymer of claim 1, wherein;
at least one P¹ comprises at least one catechol residue; and
the at least one catechol residue comprises 3,4-dihydroxyphenylalanine.

14. The polymer of claim 1, wherein the at least one P¹ further comprises at least one lysine residue.

15. The polymer of claim 1, wherein m is an integer from 2 to 100, n is 0, and at least 50% of all instances of P¹ on a number basis comprise a sequence having 75% or greater sequence identity of a tandem repeat peptide sequence found in a natural protein or natural peptide.

16. The polymer of claim 1, wherein m is an integer from 2 to 100, n is 0, and each instance of P¹ is the same and comprises a sequence having 75% or greater sequence identity of a tandem repeat peptide sequence found in a natural protein or natural peptide.

17. (canceled)

18. The polymer of claim 1, wherein at least one P¹ comprises a sequence having a helix-loop-helix motif.

19. The polymer of claim 1, wherein:
at least one P¹ comprises a sequence having 75% or greater sequence identity of a tandem repeat peptide sequence found in a natural protein or natural peptide; and
the tandem repeat peptide sequence is found in an ankyrin protein.

20. The polymer of claim 1, wherein at least one P¹ comprises a sequence having 75% or greater sequence identity of
SEQ ID NO: 2
(DKXGRTPLHLAARXGHLEVVKLLLXXGADVNAK)
or
SEQ ID NO: 4
(DXXGXTPLHLAAXXGHLEIVEVLLXAGADVNAX),
wherein each X independently is a hydrophilic amino acid residue.

21. The polymer of claim 1, wherein
at least one P¹ comprises a sequence having 75% or greater sequence identity of SEQ ID NO: 3 (DKNGRTPLHLAARNGHLEVVKLLLEAGADVNAK).

22. (canceled)

23. The polymer of claim 1, wherein at least one P¹ comprises a sequence having 75% or greater sequence identity of:
SEQ ID NO: 5
(DKNGRTPLHLAARNGHLEIVEVLLEAGADVNAK)[[.]];
SEQ ID NO: 6
(DAAREGFLDTLVVLHRAGAR);
SEQ ID NO: 7
(FLDTLVVLHR);
SEQ ID NO: 8
(ALPNAPNSYGRRPIQVMMMGSARVAELLLLHGAE);
SEQ ID NO: 9
(PNCADPATLTRPVHDAAREGELDTLVVLHRA);
or
SEQ ID NO: 15
(PNMADPATLTRPVHDAAREGFLDTLVVLHRA).

24. (canceled)

25. (canceled)

26. (canceled)

27. (canceled)

28. (canceled)

29. (canceled)

30. The polymer of claim 1, wherein at least one P¹ comprises a sequence having 75% or greater sequence identity of:
SEQ ID NO: 10
(GSGSGS)[[.]];
SEQ ID NO: 11
(KIABAVBLBAEE);
SEQ ID NO: 12
(SWTWENGKWTWK);
SEQ ID NO: 13
(GSGSGRGSGSGE);
or
SEQ ID NO: 14
(NGRTPLHLAARNGHLEVVKLLLEAGADVNAKDK)

31. (canceled)

32. (canceled)

33. (canceled)

34. (canceled)

35. The polymer of claim 1, wherein the polymer comprises at least one of the following properties:
each P¹ independently comprises from 5-100 amino acid residues;
m is an integer from 2 to 100;
n is an integer from 0 to 100;
m is an integer from 2 to 100, n is 0, and at least one instance of P¹ is different from another instance of P¹;
the degree of polymerization of polymer backbone subunits is an integer from 2 to 200, or 2 to 50;
a molecular weight of from 1 kDa to 1,000 kDa;
a peptide density of at least 50%, as defined by equation m/(m+n)×100;
a combination thereof; or
any combination thereof.

36. The polymer of claim 1, wherein at least one P¹ comprises a sequence comprising gaps between one or more amino acid residues, wherein the gaps comprise up to five amino acid residues or other spacer molecules.

37. (canceled)

38. The polymer of claim 1, wherein at least one P¹ is characterized by a substructure (S3):

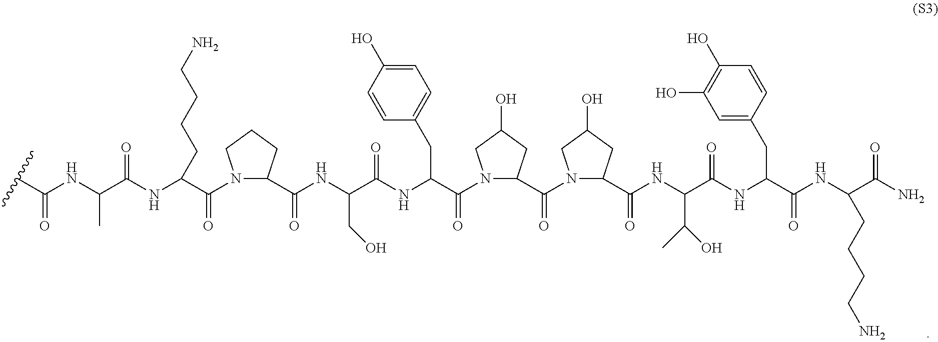 [SMILES: *C(=O)NC(C)C(=O)NC(CCCCN)C(=O)N1CCCC1C(=O)NC(CO)C(=O)NC(Cc1ccc(O)cc1)C(=O)N1CC(O)CC1C(=O)N1CC(O)CC1C(=O)NC(C(=O)NC(Cc1ccc(O)c(O)c1)C(=O)NC(CCCCN)C(N)=O)C(C)O]

39. The polymer of claim 1, wherein each of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R³, —CONR⁴R⁵, —COR⁶, —SOR⁷, —OSR⁸, —SO₂R⁹, —OR¹⁰, —SR¹¹, —NR¹²R¹³, —NR¹⁴COR¹⁵, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R³-R¹⁵ independently is H, C₅-C₁₀ aryl, or C₁-C₁₀ alkyl.

40. A polymer characterized by a formula (FX2): 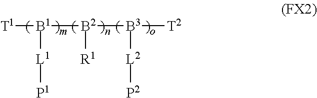
wherein:
each L¹ and L² is independently a linking groups;
each B¹, B², and B³ is independently a polymer backbone subunit;
m is an integer selected from the range of 2 to 1000;
n is an integer selected from the range of 0 to 1000;
o is an integer from 0 to 1000;
each P¹ is a peptide;
at least one P¹ comprises: (i) a sequence having at least 75% or greater sequence identity of a tandem repeat peptide sequence found in a natural protein or natural peptide and/or (ii) at least one catechol residue;
each T¹ and T² is independently a polymer backbone terminating group that can be the same or different;
each instance of P¹ is the same;
each P² is a peptide;
each instance of P² is the same or different, and each P² is different from P¹;
optionally P² comprises: (i) a sequence having at least 75% or greater sequence identity of a tandem repeat peptide sequence found in a natural protein or natural peptide and/or (ii) at least one catechol residue;
each instance of B¹, B², B³, L¹, L², and R¹ is the same as or different from any other instance of B¹, B², B³, L¹, L², and R¹, respectively;
each connecting line in formula (FX2) represents a covalent linkage comprising at least one of a single bond, a double bond, one or more atoms, or any combination thereof, optionally, for example, each connecting line represents a single bond or double bond;
when n and/or o is an integer from 1 to 1000, the polymer is a block copolymer or a statistical copolymer; and
at least one of P¹ and/or at least one of P² independently comprise a sequence having at least 75% or greater sequence identity of any one of SEQ ID NO: 1-16.

41. The polymer of claim 40 wherein P² comprises: (i) a sequence having at least 75% or greater sequence identity of a tandem repeat peptide sequence found in a natural protein or natural peptide or a modification thereof and (ii) at least one catechol residue.

42. The polymer of claim 40, wherein each instance of L¹ and L² independently comprises a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, wherein each L¹ is configured with one or more suitable functional groups to covalently attach B¹ with P¹, and wherein each L² is configured with one or more suitable functional groups to covalently attach B³ with P².

43. The polymer of claim 40, wherein at least one of B¹, B², or B³ comprises a polymerized monomer comprising an unsaturated monomer.

44. (canceled)

45. The polymer of claim 40, wherein each instance of a substructure (Sla): 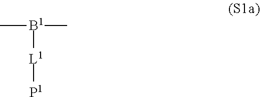
in formula (FX2) is independently characterized by a substructure (S1b), (S1c), (S1d), or (S1e): 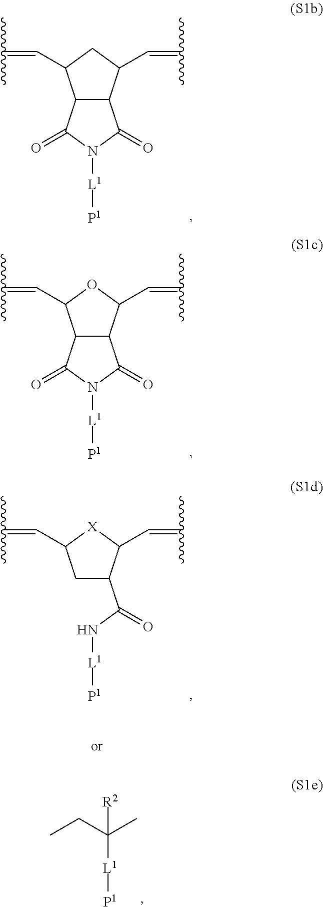
wherein L¹ is optionally present, R² is H or C₁-C₃ alkyl, and X is CH₂ or O.

46. The polymer of claim 40, wherein each instance of a substructure (S2a): 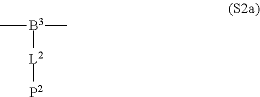
in formula (FX2) is independently characterized by a substructure (S2b), (S2c), (S2d), or (S2e): 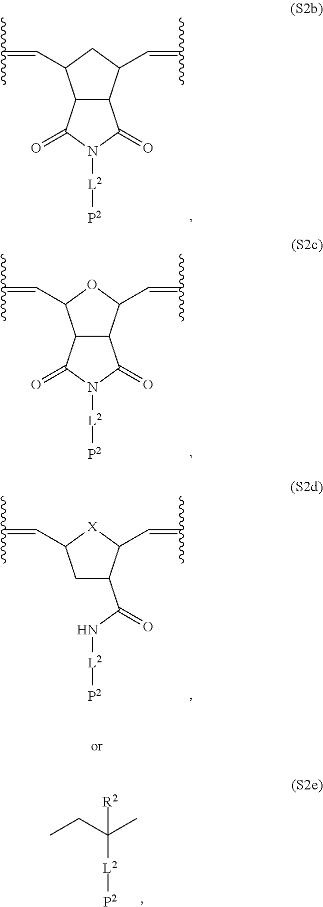
wherein L² is optionally present, R² is H or C₁-C₃ alkyl, and X is CH₂ or O.

47. The polymer of claim 1, wherein, when subjected to an atomic force microscopy adhesion test, the polymer has an adhesion force of at least 100 pN.

48. (canceled)

49. An adhesive composition comprising the polymer of claim 1, wherein the adhesive composition is a dental adhesive, a surgical adhesive, an underwater adhesive, a subterranean adhesive, a marine adhesive, or any combination thereof.

50. (canceled)

51. A method of adhering a first substrate to a second substrate, the method comprising:
disposing the polymer of claim 1 between the first substrate and the second substrate;
wherein the polymer optionally is in a form of an adhesive composition.

52. (canceled)

53. (canceled)

54. (canceled)

55. (canceled)

56. A method of making the polymer of claim 1, the method comprising:
synthesizing at least one P¹;
capping the at least one P¹ peptide at a terminal end with a polymerizable monomer that, once polymerized, becomes polymer backbone subunit B¹, thereby forming a polymerizable P¹ monomer;
polymerizing the polymerizable P¹ monomer.

57. (canceled)

58. A method comprising use of the polymer of claim 1 as a therapeutic, as an antibody mimetic, as a protein-protein interaction (PPI) disrupting agent, or any combination thereof.

59. A method comprising use of the polymer of claim 1 as a therapeutic, further comprising administering a therapeutically effective amount of the polymer and a pharmaceutically acceptable excipient to a subject, cell, or tissue in need thereof, optionally wherein the polymer is effective for treating macular degeneration, a tumor (e.g., cancer, such as breast cancer), or a symptom or manifestation thereof.

60. The polymer of claim 1, wherein each instance of L¹ independently comprises a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20, wherein each L¹ is configured with one or more suitable functional groups to covalently attach B¹ with P¹.

61. The polymer of claim 1, wherein at least one of B¹ or B² comprises a polymerized monomer comprising an unsaturated monomer.

62. The polymer of claim 1, wherein each instance of a substructure (S1a): 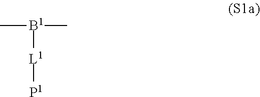
in formula (FX1) is independently characterized by a substructure (S1b), (S1c), (S1d), or (S1e): 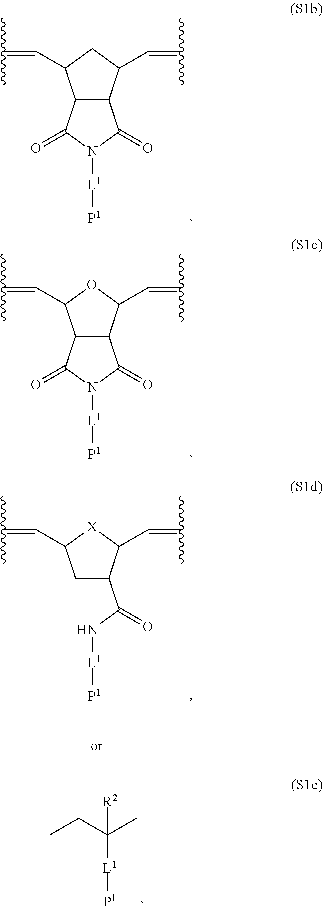
wherein L¹ is optionally present, R² is H or C₁-C₃ alkyl, and X is CH₂ or O.

63. A method of adhering a first substrate to a second substrate, the method comprising:
disposing the polymer of claim 1 between the first substrate and the second substrate;
wherein the first substrate and the second substrate independently comprise a bone, a tooth, a tissue, a cell, glass, a metal, ceramic, rock, mineral, cement, concrete, wood, plastic, polymer, paper, cardboard, or any combination thereof; and
wherein the polymer optionally is in a form of an adhesive composition.


SEQUENCE LISTING (16 sequences)

SEQ ID NO: 1 - protein, 10 aa, synthetic construct
AKXSXXXTXK

SEQ ID NO: 2 - protein, 33 aa, synthetic construct
DKXGRTPLHLAARXGHLEVVKLLLXXGADVNAK

SEQ ID NO: 3 - protein, 33 aa, synthetic construct
DKNGRTPLHLAARNGHLEVVKLLLEAGADVNAK

SEQ ID NO: 4 - protein, 33 aa, synthetic construct
DXXGXTPLHLAAXXGHLEIVEVLLXAGADVNAX

SEQ ID NO: 5 - protein, 33 aa, synthetic construct
DKNGRTPLHLAARNGHLEIVEVLLEAGADVNAK

SEQ ID NO: 6 - protein, 20 aa, synthetic construct
DAAREGFLDTLVVLHRAGAR

SEQ ID NO: 7 - protein, 10 aa, synthetic construct
FLDTLVVLHR

SEQ ID NO: 8 - protein, 34 aa, synthetic construct
ALPNAPNSYGRRPIQVMMMGSARVAELLLLHGAE

SEQ ID NO: 9 - protein, 31 aa, synthetic construct
PNCADPATLTRPVHDAAREGFLDTLVVLHRA

SEQ ID NO: 10 - protein, 6 aa, synthetic construct
GSGSGS

SEQ ID NO: 11 - protein, 12 aa, synthetic construct
KIABAVBLBAEE

SEQ ID NO: 12 - protein, 12 aa, synthetic construct
SWTWENGKWTWK

SEQ ID NO: 13 - protein, 12 aa, synthetic construct
GSGSGRGSGSGE

SEQ ID NO: 14 - protein, 33 aa, synthetic construct
NGRTPLHLAARNGHLEVVKLLLEAGADVNAKDK

SEQ ID NO: 15 - protein, 31 aa, synthetic construct
PNMADPATLTRPVHDAAREGFLDTLVVLHRA

SEQ ID NO: 16 - protein, 10 aa, synthetic construct
AKPSYPPTYK